(19) United States
(12) Patent Application Publication
(10) Pub. No.: US 2025/0218535 A1
(43) Pub. Date: Jul. 3, 2025

(54) IDENTIFICATION OF CLONAL NEOANTIGENS AND USES THEREOF

(71) Applicant: Achilles Therapeutics UK Limited, London (GB)

(72) Inventors: Fong Chun Chan, London (GB); Rachel Rosenthal, London (GB)

(21) Appl. No.: 18/851,405

(22) PCT Filed: Apr. 5, 2023
(86) PCT No.: PCT/EP2023/059033

§ 371 Date: Sep. 26, 2024

(30) Foreign Application Priority Data
Apr. 7, 2022 (GB) 2205147.8

(51) Int. Cl. G16B 20/20; G16B 5/20; G16B 30/00; G16B 40/20

(52) U.S. Cl. CPC G16B 20/20; G16B 5/20; G16B 30/00; G16B 40/20

(57) ABSTRACT

The present disclosure provides methods of determining whether a tumour-specific mutation is likely to be clonal in a subject. The method comprise determining, by a processor, a metric indicative of the probability that the mutation is clonal by combining evidence from sequence data obtained from the subject and an indication of whether the mutation is associated with one or more predetermined mutational signatures. The methods may be used to identify clonal mutations and clonal neoantigens in a subject, and to design immunotherapies that target clonal neoantigens. Related methods, systems and products are also described.

### FIELD OF THE DISCLOSURE

The present disclosure relates to methods for determining whether a tumour-specific mutation is likely to be clonal and for identifying clonal neoantigens derived from tumour-specific mutations present in a tumour. The present disclosure also relates to methods and compositions for the treatment of cancer which make use of or target identified clonal neoantigens.

### BACKGROUND

Cancer cells are known to acquire many mutations from initial cell transformation, through various endogenous (e.g. DNA mismatch repair deficiency) and exogenous (e.g. UV exposure) mutational processes. As a result, tumours often comprise multiple genotypically distinct, related populations (or clones), each of which has a mutated genome, resulting in a complex genomic picture.

In recent years, mutational signatures (see e.g. Alexandrov et al. 2013) have emerged as a way to characterise the patterns of genetic alterations observed in cancers, providing indications of mutational processes underlying the complex genomic picture observable in a cancer genome. This information can in turn be used to design therapeutic strategies for example when the presence of particular mutational signatures in a cancer is indicative of a targetable pathway defect (see e.g. Ma et al. 2018, Nik-Zainal et al. 2016). Thus, approaches to identify the presence of mutational signatures in cancer have been developed and include MMSig (Rustad et al., 2021) and deconstructSigs (Rosenthal et al., 2016).

Another aspect of cancer genomic heterogeneity, the tumour's clonal composition, is also important in a therapeutic context. Indeed, targeting mutations that are present only in subsets of the tumour cell population (also referred to as “subclonal” mutations) may be associated with limited clinical benefit as the therapy only targets part of the population and a high likelihood of relapse or metastasis as unaffected clones remain able to proliferate. Instead, it is increasingly believed that targeting clonal neoantigens (antigens expressed as a result of the presence of mutations that are present in all tumor cells) or combining multiple targeted therapies may be necessary to effectively control a tumour (McGranahan et al., 2015). Additionally, clonal neoantigen burden is known to be associated with prognosis in at least some cancers, and with sensitivity to treatment with checkpoint inhibitors (McGranahan et al., 2016; Litchfield et al., 2021). Consequently, methods to identify clonal neoantigens have been described, including the methods described in WO 2016/16174085, Landau et al. (2013), Roth et al. (2014), McGranahan et al. (2016).

### SUMMARY

The present inventors postulated that at least some mutational signatures, which capture the activity of mutational processes in cancers, may be more active at different times during the cancer evolution process. Thus, the inventors postulated that if that was the case and such information about the interplay between mutational signatures and cancer transformation/evolution could be integrated in the process of identifying clonal neoantigens, this could potentially enhance our ability to identify these very clinically relevant cancer antigens. Indeed, the present inventors identified that previously described methods to identify clonal neoantigens typically make very little or no use of the processes involved in cancer cell transformation and evolution. Thus, the present inventors investigated whether evidence of mutational signatures activity being associated with clonality of neoantigens could be identified. Having established that this was the case, they set out to design a new framework allowing the integration of this information in a process for identifying clonal neoantigens. Finally, they tested whether this would result in an improvement in the identification of clonal neoantigens compared to a situation where no such aetiology information is taken into account. They showed that integrating this knowledge into a process for identifying clonal neoantigens results in a significant improvement in terms of the ability to identify true clonal neoantigens that result from mutational processes that are associated with cancer transformation and/or evolution.

Thus, according to a first aspect, there is provided a method of determining whether one or more tumour-specific mutations are likely to be clonal in the subject, the method comprising for each of the one or more tumour-specific mutations, determining a metric indicative of the probability that the tumour-specific mutation is clonal by combining evidence from sequence data obtained from the subject and an indication of whether the mutation is associated with one or more predetermined mutational signatures. The present inventors have identified that knowledge about the mutational signatures that are associated with a mutation can provide an indication of the likelihood that the mutation is clonal. Without wishing to be bound by theory, this is because the inventors postulated that different mutational processes may be active at different times during tumour evolution, such that mutations that can be associated with specific signatures could be more or less likely to be clonal if the signature activity did show such a temporal pattern of activity in the tumour.

The method may comprise identifying one or more tumour-specific mutations in the subject. Thus, also described herein according to the present aspect is a method of identifying one or more clonal tumour-specific mutations in a subject, the method comprising: identifying one or more tumour-specific mutations in the subject; for each of the one or tumour-specific mutations, determining a probability that the tumour-specific mutation is clonal by combining evidence from sequence data obtained from the subject and an indication of whether the mutation is associated with one or more predetermined mutational signatures; and identifying a tumour-specific mutation of the one or more tumour-specific mutations as a clonal tumor-specific mutation if the mutation satisfies one or more criteria at least one of which applies to the determined metric.

The methods of the present aspect may have any one or more of the following optional features.

The step of identifying one or more tumour-specific mutations may comprise receiving a list of previously identified tumour-specific mutations.

The metric indicative of the probability that a mutation is clonal may be a posterior probability. The inventors have further identified that any method for determining whether a mutation is likely to be clonal that uses Bayesian inference could reflect such knowledge as part of the prior used to determine the probability of a mutation being clonal. The posterior probability may be a posterior probability that the mutation is clonal or a posterior probability of one or more cancer cell fractions for the mutation.

Determining a metric indicative of the probability that the mutation is clonal may comprise determining a posterior probability based on: a prior probability of the mutation being clonal that depends on the indication of whether the mutation is associated with the one or more predetermined mutational signatures, and a probability of observing the sequence data if the mutation is clonal, non-clonal and/or has a particular cancer cell fraction. The probability of observing the sequence data may comprise probabilities of observing the sequence data if the tumour-specific mutation is (i) clonal and (ii) non-clonal, in view of a tumour fraction for each of the one or more samples and one or more candidate joint genotypes each comprising a genotype at the location of the tumour-specific mutation for a normal population, a reference tumour population that does not comprise the tumour-specific mutation and a variant tumour cell population that comprises the tumour-specific mutation.

The method may further comprise identifying one or more tumour-specific mutations in the subject. The method may further comprise determining the metric indicative of the probability that a mutation is clonal for each of the one or tumour-specific mutations. The method may further comprise identifying a tumour-specific mutation of the one or more tumour-specific mutations as a clonal tumour-specific mutation if the mutation satisfies one or more criteria at least one of which applies to the determined metric. The step of identifying one or more tumour-specific mutations may comprise receiving a list of previously identified tumour-specific mutations. Identifying one or more tumour-specific mutations in the subject may be performed using sequence data from one or more samples from the subject comprising tumour genetic material and sequence data from one or more germline samples from the subject, such as by comparing said sequence data. Identifying one or more tumour-specific mutations in the subject may comprise aligning sequence data from at least one sample comprising tumour genetic material to a reference sequence and identifying positions where the sequence of the sample differs from the reference sequence. The method may further comprise aligning sequence data from at least one germline sample to the reference sequence and identifying positions where the sequence of the sample comprising tumour genetic material differs from the germline sample.

The method may further comprise providing sequence data from one or more samples from the subject. The step of providing sequence data from one or more samples from the subject may comprise or consist of receiving sequence data from a user (for example through a user interface), from one or more computing device(s), or from one or more data stores or databases. The step of providing sequence data may comprise sequencing (or otherwise determining the sequence composition of genomic material present in a sample) one or more samples from the subject comprising tumour genetic material. The method may further comprise sequencing (or otherwise determining the sequence composition of genomic material present in a sample) one or more germline samples from the subject. The method may further comprise obtaining, from the subject, one or more samples comprising tumour genetic material and optionally one or more germline samples.

The method may further comprise providing to a user, for example through a user interface, the determined metric indicative of the probability that a mutation is clonal, the probability of the tumour-specific mutation being clonal, the probability(ies) of the tumor-specific mutation having a particular cancer cell fraction, the indication of whether the mutation is associated with one or more predetermined mutational signatures, the a prior probability of the mutation being clonal, and/or any value derived therefrom or associated therewith. For example, the method may comprise providing a “clonal status” flag or value based on the determined metric indicative of the probability of the tumour-specific mutation being clonal. As another example, the method may comprise providing information identifying the mutation (such as e.g. the sequence of the mutation and its genomic location).

The indication of whether the mutation is associated with a mutational signature may comprise a weight obtained from a mutational profile for the subject, that quantifies the probability that said mutation was generated by said mutational signature. The weight may be quantified by multiplying the mutational signature proportion for the mutational class to which the mutation belongs by a sample weight quantifying the contribution of the mutational signature to the mutational profile. The indication of whether the mutation is associated with a mutational signature may further comprise a confidence interval around said weight. The method may further comprise obtaining a mutational profile for the subject. The method may further comprise obtaining a sample weight for each of the one or more predetermined mutational signatures, wherein the sample weight represents the contribution of the respective mutational signature to the mutational profile. The sample weights may be obtained using a method selected from: mmsig, deconstructsigs, sigLASSO and sigprofiler. The sample weights may be mutational signature exposures. When a confidence interval around the weights is obtained, the method may comprise using a neutral prior probability for a mutation if the confidence intervals associated with the weights indicate that no mutational signature can be confidently assigned to the mutation.

A prior probability of the mutation being clonal may be obtained as the output of a model that predicts the prior probability of the mutation being clonal using inputs comprising the indication of whether the mutation is associated with each of the mutational signatures. The model may be a linear model, a logistic regression model or a simple linear model. The model may be trained or fitted using training data comprising, for a plurality of mutations with known clonal status, indications of whether the mutations are associated with each of the mutational signatures. The indications of whether the mutations are associated with each of the mutational signatures may comprise weights obtained from a plurality of training mutational profiles, that quantify the probability that each mutation in the training mutational profiles was generated by a respective mutational signature. The indications of whether the mutations are associated with each of the mutational signatures may further comprise confidence intervals around these weights. A likelihood of clonality may be associated with each of the predetermined mutational signatures from this information by assigning each mutation in the training data to a particular mutational signature, and quantifying the proportion of clonal vs non clonal mutations assigned to each mutational signature. A mutation may be assigned to the mutational signature that has the highest weight. A mutation may be assigned to the mutational signature that has the highest weight provided that the confidence interval around the weight also satisfies one or more criteria. The confidence interval may be obtained by bootstrapping. The confidence interval may be a 90% confidence interval, a 95% confidence interval, or a 98% confidence interval. The model inputs may further comprise one or more predictive variables selected from: variables associated with the ploidy of the mutation, variables associated with the gene in which the mutation is present, variables associated with the subject, variables associated with the tumour. The variables associated with the ploidy of the mutation may comprise a genome doubling status. The variables associated with the gene may comprise a driver gene status. The variables associated with the subject may comprise an ethnicity status. The variables associated with the subject may comprise an age of the subject. The variables associated with the tumour may comprise a cancer type or subtype. The model may be a logistic regression model that predicts the log odds of a mutation being clonal as a function of respective weights obtained from a mutational profile for the subject, that quantify the probability that said mutation was generated by said mutational signature. The model may have been trained using training data comprising weights (Xn) obtained for each of a plurality of mutations from a plurality of training mutational profiles and respective clonal status for each of the plurality of mutations. The logistic regression model may comprise a coefficient βn for each of the predetermined mutational signatures and training the logistic regression model comprises identifying the value of coefficients (β₀, βₙ) of the logistic regression model. The present inventors have identified that such a model advantageously captured the prior knowledge available from mutational signatures while being extensible to include other predictive variables. Alternatively, the model may be a linear model that predicts a likelihood of clonality as a weighted sum of likelihood of clonality associated with each of the one or more mutational signatures and respective weights obtained from a mutational profile for the subject, that quantify the probability that said mutation was generated by said mutational signature. Thus, the model may be a linear model that predicts the probability of a mutation being clonal as a function of respective weights obtained from a mutational profile for the subject, that quantify the probability that said mutation was generated by said mutational signature and a signature specific probability of clonality for each mutational signature. The signature specific probability of clonality may be obtained using training data comprising weights (αn=Xn) obtained for each of a plurality of mutations from a plurality of training mutational profiles and respective clonal status for each of the plurality of mutations. Obtaining the signature specific probability of clonality for a mutational signature may comprise assigning a single mutational signature to each of a set of mutations in a plurality of training mutational profiles and determining the proportion of mutations assigned to the mutational signature that are clonal. Such a model was found to perform satisfactorily at capturing the prior knowledge available from mutational signatures. Training of a logistic regression model may be performed using a maximum likelihood algorithm. Training the logistic regression model may further comprise identifying the value of confidence intervals for the coefficients (β0—where β0 is the intercept of the model, βn) of the logistic regression model. The confidence intervals can be used to identify mutational signatures that are associated with clonality in the training samples. This may be used to select predetermined mutational signatures for inclusion in a final model to be deployed. The model of any embodiment may be specific to a particular cancer type.

The model may be trained using training data derived from a particular cancer type. The method may use a predetermined set of signatures adapted for the cancer type of the subject. The present inventors have identified that the mutational signatures that are significantly associated with clonality may depend on the cancer type such that disease specific models are likely to be more informative. The predetermined set of signatures may be selected from a set of candidate signatures for example based on the confidence intervals or other measures of statistical confidence associated with an association between the activity of a candidate mutational signature and clonality. For example, the confidence interval around a coefficient βn associated with the candidate signature in a logistic regression model as described herein may be used to determine whether a candidate mutational signature is to be used in a method as described herein. The predetermined set of signatures or the set of candidate signatures may be selected from a reference set of signatures (e.g. COSMIC signatures) based on associations between signatures and cancer types. For example, the predetermined set of signatures or the set of candidate signatures for a particular cancer type may be selected as one or more signatures that are associated with (i.e. extracted from or overrepresented in) samples from the same or a similar cancer type in a reference set of signatures (e.g. COSMIC signatures).

The method may further comprise obtaining a model that predicts the prior probability of the mutation being clonal using inputs comprising the indication of whether the mutation is associated with each of the mutational signatures. Obtaining a model may comprise training a model using training data comprising, for a plurality of mutations with known clonal status, indications of whether the mutations are associated with each of the mutational signatures.

The predetermined mutational signatures may be consensus mutational signatures. The predetermined mutational signatures may be obtained from a mutational signatures database. The predetermined mutational signatures may be mutational signatures that are likely to be active in the tumour of the subject. The predetermined mutational signatures may be mutational signatures associated with a known aetiology. The predetermined mutational signatures may be mutational signatures that are associated with a mutational process that is active in at least some tumours of the same type as the tumour in the subject. The method may further comprise selecting the predetermined signatures. The subject may be a lung cancer subject or a melanoma subject. The mutational signatures may be selected from COSMIC signatures 1, 2, 4, 5, 6, 7, 11, 13 and 17.

The sequence data may comprise sequencing reads. The sequence data may comprise a count of reads supporting the mutated allele, a count of reads supporting the germline allele(s), and/or the total count of reads, at the genomic location of the tumour-specific mutation.

Thus, also described herein is a method of providing a tool for determining whether a tumour-specific mutation is likely to be clonal in a subject, the method comprising: training a model that predicts the prior probability of a mutation being clonal using inputs comprising an indication of whether the mutation is associated with each of a set of predetermined mutational signatures, using training data comprising, for a plurality of mutations with known clonal status, indications of whether the mutations are associated with each of the mutational signatures. The method of the present aspect may include any of the features described in relation to the previous aspect.

According to a further aspect, there is provided a method of identifying one or more clonal neoantigens in a subject, the method comprising: identifying a plurality of tumour-specific mutations in the subject; determining whether one or more of the tumour-specific mutations is likely to be clonal in the subject using the method of any embodiment of the preceding aspect; and determining whether one or more of the tumour-specific mutations is likely to give rise to a neoantigen. A clonal neoantigen may be a tumour-specific mutation that satisfies one or more predetermined criteria on whether the tumour-specific mutation is likely to be clonal and one or more criteria on whether the tumour-specific mutation is likely to give rise to a neoantigen. Also described according to the present aspect is a method of identifying one or more clonal neoantigens in a subject, the method comprising: identifying, by a processor using sequence data from one or more samples from said subject, a plurality of tumour-specific mutations in the subject; determining, by a processor whether one or more of the tumour-specific mutations is likely to be clonal in the subject using the method of any preceding claim; and selecting, by said processor, one or more of the tumour-specific mutations as candidate clonal neoantigens, wherein a candidate clonal neoantigen is a tumour-specific mutation that satisfies at least one or more predetermined criteria on whether the tumour-specific mutation is likely to be clonal and optionally one or more criteria on whether the tumour-specific mutation is likely to give rise to a neoantigen.

The method of the present aspect may have any one or more of the following features.

A clonal neoantigen may be a tumour-specific mutation that satisfies at least a criterion selected from: having a probability of being clonal above a predetermined threshold, having a probability of being clonal that is above a threshold set adaptively to select a predetermined number of tumour-specific mutations with the highest probabilities of being clonal amongst the tumour-specific mutations for which a probability was determined, and having a probability of being clonal that is above a threshold set adaptively to select a predetermined top percentile of tumour-specific mutations amongst the tumour-specific mutations for which a probability was determined. Thus, the one or more predetermined criteria on whether the tumour-specific mutation is likely to be clonal may be selected from: the mutation having a likelihood of being clonal above a predetermined threshold, the mutation having a likelihood of being clonal that is above a threshold set adaptively to select a predetermined number of tumour-specific mutations with the highest likelihoods of being clonal amongst the tumour-specific mutations for which a likelihood was determined, and having a likelihood of being clonal that is above a threshold set adaptively to select a predetermined top percentile of tumour-specific mutations amongst the tumour-specific mutations for which a likelihood was determined.

A clonal neoantigen may be a tumour-specific mutation that satisfies at least a criterion selected from: being associated with an expression product that is expressed in tumour cells, being predicted to result in a protein or peptide that is not expressed in the normal cells of the subject, being predicted to result in at least one peptide that is likely to be presented by an MHC molecule, being predicted to result in at least one peptide that is likely to be presented by an MHC allele that is known to be present in the subject, and being predicted to result in a protein or peptide that is immunogenic. For example, a clonal neoantigen may be a tumour-specific mutation that satisfies a criterion that it is predicted to result in a change in the sequence of a protein (e.g. because it is coding, because it affects a splice site, because it results in a truncated peptide, etc.), thus resulting in a protein or peptide that may not be expressed in the normal cells of the subject. Whether or not this is the case may further be confirmed for example by comparison with a predicted normal proteome of the subject. Thus, the one or more criteria on whether the tumour-specific mutation is likely to give rise to a neoantigen may be selected from: the mutation being associated with an expression product that is expressed in tumour cells, the mutation being predicted to result in a protein or peptide that is not expressed in the normal cells of the subject, the mutation being predicted to result in at least one peptide that is likely to be presented by an MHC molecule, the mutation being predicted to result in at least one peptide that is likely to be presented by an MHC allele that is known to be present in the subject, and the mutation being predicted to result in a protein or peptide that is immunogenic.

The method may further comprise identifying one or more peptides associated with the one or more clonal neoantigens (i.e. one or more peptide sequences that are predicted to be present in the tumour cells as a consequence of the presence of the tumour-specific mutation, where the tumour-specific mutation satisfies one or more criteria (related to likelihood of clonality and likelihood of giving rise to a clonal neoantigen) as described above.

As the skilled person understands, the complexity of the operations described herein (due at least to the complexity of obtaining posterior probabilities requiring numerical integration as described herein, and the amount of data that is typically generated by sequencing genomic DNA) are such that they are beyond the reach of a mental activity. Thus, unless context indicates otherwise (e.g. where sample preparation or acquisition steps are described), all steps of the methods described herein are computer implemented.

According to a further aspect, there is provided a method of providing a prognosis for a subject that has been diagnosed as having cancer, the method comprising identifying a plurality of tumour-specific mutations in one or more samples from the subject and determining the likelihood of each of the tumour-specific mutations being clonal using the method of any embodiment of the first aspect.

The method may further comprise classifying the subject as having high clonal neoantigen burden vs low clonal neoantigen burden depending at least in part on the proportion of tumour-specific mutations that have a probability of being clonal above a predetermined threshold, wherein subjects with high clonal neoantigen burden have an improved prognosis compared to subjects with a low clonal neoantigen burden.

According to a further aspect, there is provided a method of providing an immunotherapy for a subject that has been diagnosed as having cancer, the method comprising: identifying one or more clonal neoantigens using a method as described herein, such as a method according to any embodiment of the second aspect; and designing an immunotherapy that targets one or more of the clonal neoantigens identified.

The method may have any one or more of the following features.

The immunotherapy that targets the one or more of the clonal neoantigens may be an immunogenic composition, a composition comprising immune cells or a therapeutic antibody. The immunogenic composition may comprise the one or more clonal of the clonal neoantigens identified (such as e.g. a neoantigen peptide or protein or a cell displaying the neoantigen), or material sufficient for expression of the one or more of the clonal neoantigens identified (e.g. a DNA or RNA molecule which encodes the neoantigen). The composition comprising immune cells may comprise T cells, B cells and/or dendritic cells. The composition comprising a therapeutic antibody may comprise one or more antibodies that recognise at least one of the one or more of the clonal neoantigens identified. An antibody may be a monoclonal antibody.

In any embodiment of any aspect, the cancer may be selected from bladder cancer, gastric cancer, oesophageal cancer, breast cancer, colorectal cancer, cervical cancer, ovarian cancer, endometrial cancer, kidney cancer (renal cell), lung cancer (small cell, non-small cell and mesothelioma), brain cancer (gliomas, astrocytomas, glioblastomas), melanoma, lymphoma, small bowel cancers (duodenal and jejunal), leukemia, pancreatic cancer, hepatobiliary tumours, germ cell cancers, prostate cancer, head and neck cancers, thyroid cancer and sarcomas. The cancer may be lung cancer. The cancer may be melanoma. The cancer may be bladder cancer. The cancer may be head and neck cancer.

In any embodiment of any aspect, the subject may be human.

Designing an immunotherapy that targets one or more of the clonal neoantigens identified may comprise designing one or more candidate peptides for each of the one or more clonal neoantigens targeted, each peptide comprising at least a portion of a clonal neoantigen targeted.

The method may further comprise obtaining the one or more candidate peptides. The method may further comprise testing the one or more candidate peptides for one or more properties. Testing may be performed in vitro or in silico. For example, the one or more peptides may be tested for immunogenicity, propensity to be displayed by MHC molecules (optionally by specific MHC molecule alleles, where the alleles may have been chosen depending on the MHC alleles expressed by the subject), ability to elicit proliferation of a population of immune cells, etc.

The method may further comprise producing the immunotherapy. The method may further comprise obtaining a population of dendritic cells that has been pulsed with one or more of the candidate peptides. The immunotherapy may be a composition comprising T cells that recognise at least one of the one or more of the clonal neoantigens identified. The composition may be enriched for T cells that target at least one of the one or more of the clonal neoantigens identified. The method may comprise obtaining a population of T cells and expanding the population of T cells to increase the number or relative proportion of T cells that target at least one of the one or more of the clonal neoantigens identified.

The method may further comprise obtaining a T cell population. A T cell population may be isolated from the subject, for example from one or more tumour samples obtained from the subject, or from a peripheral blood sample or a sample from other tissues of the subject. The T cell population may comprise tumour infiltrating lymphocytes. T cells may be isolated using methods which are well known in the art. For example, T cells may be purified from single cell suspensions generated from samples on the basis of expression of CD3, CD4 or CD8. T cells may be enriched from samples by passage through a Ficoll-paque gradient.

The method may further comprise expanding the T cell population. For example, T cells may be expanded by ex vivo culture in conditions which are known to provide mitogenic stimuli for T cells. By way of example, the T cells may be cultured with cytokines such as IL-2 or with mitogenic antibodies such as anti-CD3 and/or CD28. The T cells may be co-cultured with antigen-presenting cells (APCs), which may have been irradiated. The APCs may be dendritic cells or B cells. The dendritic cells may have been pulsed with peptides containing one or more of the identified neoantigens as single stimulants or as pools of stimulating neoantigen peptides. Expansion of T cells may be performed using methods which are known in the art, including for example the use of artificial antigen presenting cells (aAPCs), which provide additional co-stimulatory signals, and autologous PBMCs which present appropriate peptides. Autologous PBMCs may be pulsed with peptides containing neoantigens as discussed herein as single stimulants, or alternatively as pools of stimulating neoantigens.

According to a further aspect, there is provided a method for expanding a T cell population for use in the treatment of cancer in a subject, the method comprising: identifying one or more clonal neoantigens using a method as described herein, such as a method according to any embodiment of the second aspect; obtaining a T cell population comprising a T cell which is capable of specifically recognising one of the identified clonal neoantigens; and co-culturing the T cell population with a composition comprising the identified clonal neoantigens.

The method may have one or more of the following features.

The T cell population obtained may be assumed to comprise a T cell capable of specifically recognising one of the identified clonal neoantigens. The method preferably comprises identifying a plurality of clonal neoantigens. The T cell population may comprise a plurality of T cells each of which is capable of specifically recognising one of the plurality of identified clonal neoantigens, and co-culturing the T cell population with a composition comprising the plurality of identified clonal neoantigens. The co-culture may result in expansion of the T cell population that specifically recognises the one or more neoantigens. The expansion may be performed by co-culture of a T cell with a neoantigen and an antigen presenting cell. The antigen presenting cell may be a dendritic cell. Thus, the expansion may be a selective expansion of T cells which are specific for the neoantigen. The expansion may further comprise one or more non-selective expansion steps.

According to a further aspect, there is provided a composition comprising a population of T cells obtained or obtainable by a method according to any embodiment of the preceding aspect.

According to a further aspect, there is provided a composition comprising a neoantigen, neoantigen specific immune cell, or an antibody that recognises a neoantigen, for use in the treatment or prevention of cancer in a subject, wherein said neoantigen has been identified as a clonal neoantigen using the methods described herein.

According to a further aspect, there is provided a composition comprising a neoantigen, neoantigen specific immune cell, or an antibody that recognises a neoantigen, wherein said neoantigen has been identified as a clonal neoantigen using the methods described herein.

According to a further aspect, there is provided a cell or population of cells expressing a neoantigen on its surface, wherein said neoantigen has been identified as a clonal neoantigen using the methods described herein.

According to a further aspect, there is provided a neoantigen, immune cell which recognises a neoantigen, or antibody which recognises a neoantigen, for use in the treatment or prevention of cancer in a subject, wherein said neoantigen has been identified as a clonal neoantigen using the methods described herein.

According to a further aspect, there is provided a use of a neoantigen, immune cell which recognises a neoantigen, or antibody which recognises a neoantigen, in the manufacture of a medicament for use in the treatment or prevention of cancer in a subject, wherein said neoantigen has been identified as a clonal neoantigen using the methods described herein.

According to a further aspect, there is provided a method of treating a subject that has been diagnosed as having cancer, the method comprising administering an immunotherapy that has been provided using the methods described herein, or a composition as described herein.

According to a further aspect, there is provided a system comprising: a processor; and a computer readable medium comprising instructions that, when executed by the processor, cause the processor to perform the steps of any method described herein, such as a method according to any embodiment of the first, second, third or fourth aspects above.

According to a further aspect, there is provided or more non-transitory computer readable media comprising instructions that, when executed by one or more processors, cause the one or more processors to perform the steps of any method described herein, such as a method according to any embodiment of the first, second, third or fourth aspects above.

According to a further aspect, there is provided a computer program comprising code which, when the code is executed on a computer, causes the computer to perform the steps of any method described herein, such as a method according to any embodiment of the first, second, third or fourth aspects above.

### BRIEF DESCRIPTION OF THE FIGURES

FIG. 1 is a flowchart illustrating schematically a method of determining the prior probability of a mutation being clonal.

FIG. 2 is a flowchart illustrating schematically an exemplary method of determining whether a tumour-specific mutation is likely to be clonal using a prior probability, and its use in identifying clonal neoantigens.

FIG. 3 is a flowchart illustrating schematically a method of providing an immunotherapy.

FIG. 4 shows an embodiment of a system for determining a prior probability of a mutation being clonal, for determining whether a tumour-specific mutation is likely to be clonal and/or for identifying clonal neoantigens and/or for providing an immunotherapy.

FIG. 5 illustrates schematically the concept of determining weights for input mutational signatures in a sample as implemented in deconstructsigs (Rosenthal et al., 2016).

FIG. 6 shows the results of an analysis of mutational signatures activity in a tumour mutation profile that has been separated between mutations present in all regions of the tumour that are analysed (ubiquitous mutations) and mutations that are only present in a subset of these regions (non-ubiquitous mutations). A-C. Results of the analysis for the ubiquitous (A) and non ubiquitous (B-C) mutations. Each panel shows the mutational profile analysed and the resulting signature weights as a pie chart. D. Schematic of the analysis.

FIG. 7 shows example results of a method of assigning mutational signature weights and confidence intervals for a single sample. A. Signature weight for the sample and bootstrap confidence intervals for each signature. B. Mutational category weight estimate and corresponding bootstrap confidence intervals obtained for this sample. C. Heatmap of mutational category weight estimates.

FIG. 8 shows the results of analysis of signature weights per sample in a cohort of lung cancer patients, separated by cancer stage. A. LUAD. B. LUSC. Each point is a signature weight for a particular sample.

FIG. 9 shows characteristics of the signature probabilities per mutational class in the samples of FIG. 8. A. histogram of the signature weight for the highest weight signature for each mutational class, across all samples and signatures. B. Boxplots of the probabilities for the highest weight signatures, separated by identity of highest weight signature.

FIG. 10 shows the number of mutations in the samples of FIGS. 8-9 that are assigned to each signature, separated by assumed clonality status. A. LUAD. B. LUSC.

FIG. 11 illustrates exemplary approaches to determine a posterior probability of a mutation being clonal based on a prior probability that is either uninformative or informative (labelled “mutation-centric”), using a logistic regression model (A) or a weighted sum (B).

FIG. 12 show the results of fitting a logistic regression model to determine a log odds of mutations being clonal in sets of tumour samples (NSCLC on the left, melanoma on the right). Each point represents the best fit estimate for the respective term in the logistic regression model, and the bars represent the confidence intervals around these.

FIG. 13 illustrates an assessment of a method of assigning clonal priors based on mutational signatures, using synthetic data. The left panel illustrates the strategy used to generate the synthetic data. The right panel illustrates resulting number of mutations in each part of the tree assigned to each mutational signature.

FIG. 14 illustrates schematically the analysis process applied to the data on FIG. 13.

FIG. 15 shows the results of the analysis of FIG. 14. A. Distribution of posterior probabilities of mutations being clonal for mutations that are specific to the trunk and subclones 1 and 2, calculated using each of (a) a logistic regression based prior, (b) a weighted clonal prior, and (c) an uninformative prior. Each plot shows results for data with a different purity, indicated in the greyed section at the top of the plot. B. Log loss for each of the plots in A.

FIG. 16 shows the results of validation using the scheme of FIG. 14 on an independent NSCLC cohort. For each sample (columns) from top to bottom (rows): smoking and quality control status; number of mutations, mutation signature exposure (relative weights), distribution of posterior probability of clonality for mutations that are ubiquitous (present in all regions samples) and non-ubiquitous (not present in all regions sampled).

FIG. 17 shows, for the data on FIG. 16, the distribution of log loss for probabilities calculated using an uninformative prior and a logistic regression informative prior (left) and the difference between these two values as a histogram with counts representing patients (right).

FIG. 18 shows the results of validation using the scheme of FIG. 14 for the TRACERx samples kept aside for validation. For each sample (columns) from top to bottom (rows): number of mutations, mutation signature exposure (relative weights), distribution of posterior probability of clonality for mutations that are clonal and sub-clonal.

FIG. 19 shows, for the data on FIG. 18, the distribution of log loss for probabilities calculated using an uninformative prior and a logistic regression informative prior (left) and the difference between these two values as a histogram with counts representing patients (right).

### DETAILED DESCRIPTION

Multiple methods have been proposed to attempt to reconstruct the clonal architecture of tumour samples (Schwartz and Schaeffer, 2017). All of these approaches use uninformative priors. For example, in WO 2016/16174085, a posterior distribution is calculated for a plurality of values of cancer cell fractions (CCF) as P(CCF)=binom(a|N, VAF(CCF)) which represents the probability of drawing “a” variant reads out of N reads at a locus, given a particular variant allele fraction in the population, where the VAF is calculated depending on the CCF, the tumour purity (p), and the copy number at the locus in the tumour (CPNₘᵤₜ) and the normal cells (CPNₙₒᵣₘ) as: VAF(CCF)=p*CCF/CPNₙₒᵣₘ (1−p)+p*CPNₘᵤₜ where p, CPNₘᵤₜ and CPNₙₒᵣₘ can be estimated for example using ASCAT (as further described below). The P(CCF) is calculated for a grid of CCF values, and a “posterior distribution” is obtained by normalisation of this. Candidate clonal mutations are selected as those mutations that have a 95% confidence interval for P(CCF)>=0.75. This method implicitly assumes an uninformative prior as the posterior distribution P(CCF) is only based on the data (i.e. equivalent to multiplication with a uniform prior). As another example, Landau et al. (2013) also compute a posterior probability over a range of 100 CCF as P(CCF)=binom(a|N,f(CCF)) assuming a uniform prior over CCF. The expected allele fraction f of a mutation present in one copy in a fraction CCF of cancer cells is provided as f(CCF)=αCCF/(2(1−α)+αq), where a is the sample purity and q is the absolute somatic-copy number, both estimated using ABSOLUTE (Carter et al. 2012). A posterior density is obtained by calculating the value of P(CCF) over a grid of 100 CCF and normalising by dividing by their sum, and mutations are classified as clonal if their posterior probability that the CCF exceeds 0.95 is above 0.5 (i.e. cumulative posterior densities above 0.95>0.5). Again, this method implicitly assumes an uninformative prior as the posterior distribution P(CCF) is only based on the data (i.e. equivalent to multiplication with a uniform prior). As yet another example, Roth e al. (2014) use a Bayesian clustering method for grouping sets of mutations into putative clonal clusters while estimating the cellular prevalences (CCF). The model outputs a posterior density for the cellular prevalence for each mutation in the input and a matrix containing the probability that any two mutations occur in the same cluster. The model uses CCF priors drawn from a Dirichlet Process prior with base measure HO˜Uniform(0,1) (i.e. this is equivalent to a Uniform(0,1) prior for p(CCF) but allows mutations to share the same prevalence). Thus, the approach computes a posterior distribution of the prevalences for a set of mutations then uses Dirichlet Process clustering to let each mutation “choose” it prevalence (essentially picking a cluster of mutations with the same prevalence). The cluster or mutations with the highest prevalence (CCF) can then be chosen as that comprising all of the mutations deemed to be clonal. As another example, WO2022/207925 describes a Bayesian approach to determine the probability of a particular mutation being clonal (P(Z=1)) as a posterior probability (p(Z=1|d_{b},d,π,t,ρ), where d_{b} is the number of reads in the sample that show the tumour-specific mutation, d is the total number of reads at the location of the tumour-specific mutation, t is a tumour fraction, ρ is a cancer cell fraction and π is a set of candidate joint genotypes). This is also described I the examples below. This posterior probability depends on the prior probability of the mutation being clonal, and the probability of observing the sequence data (also referred to as the “likelihood” of observing the sequence data, or simply the “likelihood” of the sequence data), if the tumour-specific mutation is (i) clonal and (ii) non-clonal, in view of a tumour fraction for each of the one or more samples and one or more candidate joint genotypes. The candidate joint genotypes comprise a genotype at the location of the tumour-specific mutation for a normal population, a reference tumour population that does not comprise the tumour-specific mutation and a variant tumour cell population that comprises the tumour-specific mutation. The prior probability is by default a Bernouilli prior that assigns a 50% weight of being clonal or non-clonal to each mutation.

By contrast, in the present disclosure, the inventors developed a framework that makes use of information about the mutational signatures active in a sample to assign a prior probability of mutations in this sample being clonal. This advantageously integrates available knowledge about mutational processes active during tumour evolution in the process for identifying clonal mutations from sequence data, thereby resulting in an enhanced ability to identify clonal mutations.

In the present disclosure, the following terms will be employed, and are intended to be defined as indicated below.

A mutational signature is a characteristic combination of mutation types that arises from one or more underlying mutational processes. Mutational processes may be endogenous (such as e.g. DNA repair pathway deficiencies) or exogenous (such as e.g. exposure to genotoxins). A set of mutational signatures (also referred to herein as a “mutational signature catalogue”) can be extracted from cohorts of samples, by identifying characteristic combination of mutation types that best explain the mutational profiles of the samples in the cohort. This process also results in the quantification of “exposures” to each of the signatures, which quantify the extent of the effect of the respective signatures on the respective mutational profiles.

A mutational signature catalogue can be extracted from a plurality of mutational profiles (also referred to herein as “mutational catalogue”) each associated with respective samples. A mutational signature catalogue can comprise mutational signatures extracted separately from a plurality of respective cohorts of mutational profiles. A mutational profile comprises the number of mutations present in a sample within each of a plurality of mutation categories (also referred to herein as “mutation classes”). A mutational signature extracted from mutational profiles comprising these categories therefore comprises a respective plurality of weights (signature weights) for the mutation classes. Each signature weight can be seen as quantifying the probability that the signature will generate a mutation in the respective mutation class. Each signature weight can be seen as the proportion of mutations generated by that signature that fall in the respective mutation class. A mutational profile can be seen as a summary of a list of mutations present in a sample, categorised according to a predetermined set of mutation categories. A list of somatic mutations or a mutational profile derived therefrom may comprise mutations of one or more types selected from: substitutions, rearrangements, deletions, and insertions (sometimes collectively referred to as “indels”). A mutational profile summarising somatic substitutions associated with a sample or a group of samples may be referred to as a “substitution profile”. Substitutions may be single nucleotide substitutions (also referred to as single base substitutions, SBS). The plurality of categories in the context of substitutions may refer to the identity of the germline and mutated bases, and/or to the context of the mutated bases (identity of the one or more nucleotides flanking the mutated bases). In particular, in the context of SBS, the plurality of categories typically refer to the identity of the germline and mutated base, and the identity of the 5′ and 3′ flanking bases. Thus, such categories may include one or more of the following categories, or categories that combine some of the following categories such as based on a common context and/or substitution: A[C>A]A, A[C>A]C, A[C>A]G, A[C>A]T, C[C>A]A, C[C>A]C, C[C>A]G, C[C>A]T, G[C>A]A, G[C>A]C, G[C>A]G, G[C>A]T, T[C>A]A, T[C>A]C, T[C>A]G, T[C>A]T, A[C>G]A, A[C>G]C, A[C>G]G, A[C>G]T, C[C>G]A, C[C>G]C, C[C>G]G, C[C>G]T, G[C>G]A, G[C>G]C, G[C>G]G, G[C>G]T, T[C>G]A, T[C>G]C, T[C>G]G, T[C>G]T, A[C>T]A, A[C>T]C, A[C>T]G, A[C>T]T, C[C>T]A, C[C>T]C, C[C>T]G, C[C>T]T, G[C>T]A, G[C>T]C, G[C>T]G, G[C>T]T, T[C>T]A, T[C>T]C, T[C>T]G, T[C>T]T, A[T>A]A, A[T>A]C, A[T>A]G, A[T>A]T, C[T>A]A, C[T>A]C, C[T>A]G, C[T>A]T, G[T>A]A, G[T>A]C, G[T>A]G, G[T>A]T, T[T>A]A, T[T>A]C, T[T>A]G, T[T>A]T, A[T>C]A, A[T>C]C, A[T>C]G, A[T>C]T, C[T>C]A, C[T>C]C, C[T>C]G, C[T>C]T, G[T>C]A, G[T>C]C, G[T>C]G, G[T>C]T, T[T>C]A, T[T>C]C, T[T>C]G, T[T>C]T, A[T>G]A, A[T>G]C, A[T>G]G, A[T>G]T, C[T>G]A, C[T>G]C, C[T>G]G, C[T>G]T, G[T>G]A, G[T>G]C, G[T>G]G, G[T>G]T, T[T>G]A, T[T>G]C, T[T>G]G, and T[T>G]T. Various mutational signature catalogues have been described, together with methods for extracting such catalogues, for example see Alexandrov et al., 2020 and Degasperi et al., 2020. Typically, these methods involve using nonnegative matrix factorization (NMF) to identify a matrix of signatures S and a matrix of exposures E such that C≈SE, where C is a matrix comprising a set of mutational profiles for a (preferably large) cohort of samples. The present disclosure is not concerned with the identification of mutational signatures and any previously identified mutational signatures may be used. For example, consensus signatures that are identified across many types of cancer are available in databases such as COSMIC (https://cancer.sanger.ac.uk/signatures/) and Signal (https://signal.mutationalsignatures.com/) and any one or more of those signatures may be used in the context of the present invention. The term “reference signature” or “consensus signature” refers to a signature that has been identified across a plurality of cancer types and/or a plurality of cohorts of samples. This is typically a signature from a curated set of signatures. For example, the signatures available in COSMIC and Signal are reference signatures. Each signature is provided as a set of weights, also referred to as “fraction of mutations” or “proportion of mutations”, comprising a weight for each of the different mutation classes analysed. These capture the probability that the mutational signature will generate mutations in each of the classes.

The methods described herein relate at least in part to determining an indication of which of the mutational signatures in a set of mutational signatures is present in the sample. This may be based on exposure of the mutational signatures in the catalogue (also referred to as “mutational signature weights” or simply “weights”). Methods for determining the exposure to a mutational signature in a mutational signature catalogue are known in the art (see e.g. Alexandrov et al., 2020; Degasperi et al., 2020; Gehring et al., 2015). In particular, the determination of the exposure to one or more mutational signatures in a set may be performed by identifying the matrix E that satisfies C≈PE where C is a mutational profile for a plurality of samples for which exposure is to be determined, P is a signature matrix comprising the one or more mutational signatures for which exposure is to be determined, and E is an exposure matrix. Further, methods for determining signature weights (corresponding to normalised exposures) for a specific subset of signatures in a single sample are also known and include sigLASSO (Li et al., 2020), deconstructSigs (Rosenthal et al., 2016), mmsig (Rustad et al., 2021), and sigprofiler (Bergstrom et al., 2019). The present disclosure is not concerned with methods for determining sample exposures or weights and any methods known in the art for doing this may be used. The term “exposure” is typically used in the context of NMF where the exposures represent the number of mutations in a mutational profile that are attributed to each of the respective signatures in a mutational signature catalogue, the mutational profile forming part of a set of mutational profiles. The term “weight” (or “sample weight”, “sample signature weight”) is typically used to refer to a value that quantifies the relative activity of a set of candidate signatures in a single sample. The set of candidate signatures may comprise all of the signatures in a mutational signature catalogue, or a subset thereof. When a subset of signatures is used, the weights for these may be determined together with a weight for an unknown category (also referred to as “other” or “other signatures” or “residual”) that captures the contribution of all signatures not in the subset. The weights may sum to 1 (with an unknown category weight if such a weight is used, such as e.g. in deconstructSigs, or without if such a weight is not used, such as e,g, in mmsig). Exposures and weights are equivalent for a single mutational profile as a number of mutations can be obtained from a weight by multiplying by the total number of mutations in the mutational profile. Thus, the two terms are used interchangeably herein unless context indicates otherwise. In the context of the present invention, a mutational signature may be considered to be active in a sample if it is associated with a non-zero weight/exposure.

As described further below, the present disclosure provides a method of assigning signature weights for individual mutations. These may be referred to as “mutation weights” or “mutation-specific signature weights”. By contrast with the exposure/weights above which quantify the relative activity of a set of mutational signatures in a profile, the mutation weights quantify the probability that each mutational signature in the set of mutational signatures is associated with a particular mutation in a particular sample. These may be obtained from the sample weights/exposure by multiplying, for each mutation class and each signature, the sample weight for the signature by the signature weight for the particular mutation class (i.e. the proportion of mutations associated with the signature that belong to the mutation class). When an exposure is used these values can be normalised by the total number of mutations in the mutational profile of the sample.

Thus, according to the present disclosure, a sample (or mutational profile associated therewith) may be associated with a set of sample weights/exposures, each of which quantify the activity in the sample of a mutational signature in a set of mutational signatures. These represent a number or proportion of mutations (across all mutation classes) attributable to each of the mutational signatures. The set of sample weights/exposures may be a vector of size s (where s is the number of mutational signatures in the sample, including an “other signatures” term if applicable). The sample is further associated with a set of mutation-specific weights, each of which quantify the probability that a mutational signature in the set of mutational signatures is associated with a particular mutation. This may be a matrix of size m×s, where m is the number of mutations in the sample or the number of mutation classes.

The present disclosure relates to the use of mutational signature information to obtain an indication of whether a mutation in a tumour is likely to be clonal, in the absence of sequence data from the tumour from which clonality can be directly estimated. Such an indication may take the form of a prior belief or prior probability. A prior probability is a probability that represent a belief about a quantity before some evidence is taken into account. In the context of the present invention, the terms “prior”, “prior probability” or “clonal prior” refer to the prior belief that a mutation is clonal, unless context indicates otherwise. For example, prior probabilities that capture assumptions of how a cancer cell fraction (number of cells showing evidence of the presence of a mutation) should behave for a truly clonal/non-clonal mutation are also used in some embodiments. These are distinct from the prior probability of a mutation being clonal, which are the primary focus of the present disclosure.

A “sample” as used herein may be a cell or tissue sample, a biological fluid, an extract (e.g. a DNA extract obtained from the subject), from which genomic material can be obtained for genomic analysis, such as genomic sequencing (e.g. whole genome sequencing, whole exome sequencing). The sample may be a cell, tissue or biological fluid sample obtained from a subject (e.g. a biopsy). Such samples may be referred to as “subject samples”. In particular, the sample may be a blood sample, or a tumour sample, or a sample derived therefrom. The sample may be one which has been freshly obtained from a subject or may be one which has been processed and/or stored prior to genomic analysis (e.g. frozen, fixed or subjected to one or more purification, enrichment or extraction steps). The sample may be a cell or tissue culture sample. As such, a sample as described herein may refer to any type of sample comprising cells or genomic material derived therefrom, whether from a biological sample obtained from a subject, or from a sample obtained from e.g. a cell line. In embodiments, the sample is a sample obtained from a subject, such as a human subject. The sample is preferably from a mammalian (such as e.g. a mammalian cell sample or a sample from a mammalian subject, such as a cat, dog, horse, donkey, sheep, pig, goat, cow, mouse, rat, rabbit or guinea pig), preferably from a human (such as e.g. a human cell sample or a sample from a human subject). Further, the sample may be transported and/or stored, and collection may take place at a location remote from the genomic sequence data acquisition (e.g. sequencing) location, and/or any computer-implemented method steps described herein may take place at a location remote from the sample collection location and/or remote from the genomic data acquisition (e.g. sequencing) location (e.g. the computer-implemented method steps may be performed by means of a networked computer, such as by means of a “cloud” provider).

A “mixed sample” refers to a sample that is assumed to comprise multiple cell types or genetic material derived from multiple cell types. Within the context of the present disclosure, a mixed sample is typically one that comprises tumour cells, or is assumed (expected) to comprise tumour cells, or genetic material derived from tumour cells. Samples obtained from subjects, such as e.g. tumour samples, are typically mixed samples (unless they are subject to one or more purification and/or separation steps). Typically, the sample comprises tumour cells and at least one other cell type (and/or genetic material derived therefrom). For example, the mixed sample may be a tumour sample. A “tumour sample” refers to a sample derived from or obtained from a tumour. Such samples may comprise tumour cells and normal (non-tumour) cells. The normal cells may comprise immune cells (such as e.g. lymphocytes), and/or other normal (non-tumour) cells. The lymphocytes in such mixed samples may be referred to as “tumour-infiltrating lymphocytes” (TIL). A tumour may be a solid tumour or a non-solid or haematological tumour. A tumour sample may be a primary tumour sample, tumour-associated lymph node sample, or a sample from a metastatic site from the subject. A sample comprising tumour cells or genetic material derived from tumour cells may be a bodily fluid sample. Thus, the genetic material derived from tumour cells may be circulating tumour DNA or tumour DNA in exosomes. Instead or in addition to this, the sample may comprise circulating tumour cells. A mixed sample may be a sample of cells, tissue or bodily fluid that has been processed to extract genetic material. Methods for extracting genetic material from biological samples are known in the art. A mixed sample may have been subject to one or more processing steps that may modify the proportion of the multiple cell types or genetic material derived from the multiple cell types in the sample. For example, a mixed sample comprising tumour cells may have been processed to enrich the sample in tumour cells. Thus, a sample of purified tumour cells may be referred to as a “mixed sample” on the basis that small amounts of other types of cells may be present, even if the sample may be assumed, for a particular purpose, to be pure (i.e. to have a tumour fraction of 1 or 100%).

The term “tumour fraction” (also sometimes referred to as “tumour purity” or simply “purity”, or aberrant cell fraction (ACF)) refers to the proportion of DNA containing cells within a mixed sample that are tumour cells, or to the equivalent proportion that is assumed to result in a particular mixture of genetic material from tumour and non-tumour cells in a sample. Methods for determining the tumour fraction in a sample are known in the art. For example, in the context of cell or tissue samples, a tumour fraction may be estimated by analysing pathology slides (e.g. hematoxylin and eosin (H&E)-stained slides or other histochemistry or immunohistochemistry slides, by counting tumour cells in one or more representative areas of a sample), or using high throughput assays such as flow cytometry. In the context of samples comprising genetic material, a tumour fraction may be estimated using sequence analysis processes that attempt to deconvolute tumour and germline genomes such as e.g. ASCAT (Van Loo et al., 2010), ABSOLUTE (Carter et al., 2012), or ichorCNA (Adalsteinsson et al., 2017).

A “normal sample” or “germline sample” refers to a sample that is assumed not to comprise tumour cells or genetic material derived from tumour cells. A germline sample may be a blood sample, a tissue sample, or a purified sample such as a sample of peripheral blood mononuclear cells from a subject. Similarly, the terms “normal”, “germline” or “wild type” when referring to sequences or genotypes refer to the sequence/genotype of cells other than tumour cells. A germline sample may comprise a small proportion of tumour cells or genetic material derived therefrom, and may nevertheless be assumed, for practical purposes, not to comprise said cells or genetic material. In other words, all cells or genetic material may be assumed to be normal and/or sequence data that is not compatible with the assumption may be ignored.

The term “sequence data” refers to information that is indicative of the presence and preferably also the amount of genomic material in a sample that has a particular sequence. Such information may be obtained using sequencing technologies, such as e.g. next generation sequencing (NGS), for example whole exome sequencing (WES), whole genome sequencing (WGS), or sequencing of captured genomic loci (targeted or panel sequencing), or using array technologies, such as e.g. copy number variation arrays, or other molecular counting assays. When NGS technologies are used, the sequence data may comprise a count of the number of sequencing reads that have a particular sequence. When non-digital technologies are used such as array technology, the sequence data may comprise a signal (e.g. an intensity value) that is indicative of the number of sequences in the sample that have a particular sequence, for example by comparison to an appropriate control. Sequence data may be mapped to a reference sequence, for example a reference genome, using methods known in the art (such as e.g. Bowtie (Langmead et al., 2009)). Thus, counts of sequencing reads or equivalent non-digital signals may be associated with a particular genomic location (where the “genomic location” refers to a location in the reference genome to which the sequence data was mapped). Further, a genomic location may contain a mutation, in which case counts of sequencing reads or equivalent non-digital signals may be associated with each of the possible variants (also referred to as “alleles”) at the particular genomic location. The process of identifying the presence of a mutation at a particular location in a sample is referred to as “variant calling” and can be performed using methods known in the art (such as e.g. the GATK HaplotypeCaller, https://gatk.broadinstitute.org/hc/en-us/articles/360037225632-HaplotypeCaller). For example, sequence data may comprise a count of the number of reads (or an equivalent non-digital signal) which match a germline (also sometimes referred to as “reference”) allele at a particular genomic location, and a count of the number of reads (or an equivalent non-digital signal) which match a mutated (also sometimes referred to as “alternate”) allele at the genomic location.

Further, sequence data may be used to infer copy number profiles along a genome, using methods known in the art. Copy number profiles may be allele specific. In the context of the present disclosure, copy number profiles are preferably allele specific and tumour/normal sample specific. In other words, the copy number profiles used in the present disclosure are preferably obtained using methods designed to analyse samples comprising a mixture of tumour and normal cells, and to produce allele-specific copy number profiles for the tumour cells and the normal cells in a sample. Allele specific copy number profiles for mixed samples may be obtained from sequence data (e.g. using read counts as described above), using e.g. ASCAT (Van Loo et al., 2010). Other methods are known and equally suitable. Preferably, within the context of the present disclosure, the method used to obtain allele-specific copy number profiles is one that reports a plurality of possible copy number solutions and an associated quality/confidence metric. For example, ASCAT outputs a goodness-of-fit metric for each combination of values of ploidy (ploidy for a whole tumour sample, not segment-specific) and purity for which a corresponding allele-specific copy number profile was evaluated. Note that the tumour-specific copy number profiles generated by such methods represent an average or summary of the entire tumour cell population (i.e. it does not account for heterogeneity within the tumour population, which is the object of the new developments described herein).

The term “total copy number” refers to the total number of copies of a genomic region in a sample. The term “major copy number” refers to the number of copies of the most prevalent allele in a sample. Conversely, the term “minor copy number” refers to the number of copies of the allele other than the most prevalent allele in a sample. Unless indicated otherwise, these terms refer to the inferred major and major copy numbers (and total copy numbers) for an inferred tumour copy number profile. The term “normal copy number” or “normal total copy number” refers to the number of copies of a genomic region in the normal cells in a sample. Normal cells typically have two copies of each chromosome (unless the cell is genetically male and the chromosome is a sex chromosome), and hence the normal copy number may in embodiments be assumed to be equal to 2 (unless the genomic region is on the X or Y chromosome and the sample under analysis is from a male subject, in which case the normal copy number may be assumed to be equal to 1). Alternatively, the normal copy number for a particular genomic region may be determined using a normal sample.

The term “log R value” (sometimes referred to as “log R”, “log RR”, “LLR”) refers to a measure of normalised total signal intensity, quantifying the total copy number at a genomic locus. In the context of the present disclosure, the term typically refers to the log R value for a sample comprising tumour genetic material, and the normalisation is typically performed by reference to a normal sample (which is preferably a matched normal sample but may also be a process-matched normal sample or other suitable normal reference sample). For example, where NGS is used, the log R may be obtained as the normalised log transform of read depth (log(read depth tumour/read depth normal)). The term “mean B allele frequency” (MBAF, also sometimes referred to as “B allele frequency” (BAF)) is a measure of normalised allelic intensity ratio at a genomic location. In the context of the present disclosure, the term typically refers to the BAF value for a sample comprising tumour genetic material, and the normalisation is typically performed by reference to a normal sample (which is preferably a matched normal sample but may also be a process-matched normal sample or other suitable normal reference sample). For example, the BAF may be obtained as the ratio of the allele frequency for the tumour allele vs the normal allele. Copy number profiles typically comprise copy number estimates over genomic regions called “segments”. Thus, the BAF and log R associated with a genomic location may refer to the BAF and log R of the segment overlapping a particular genomic location (such as e.g. the genomic location of a mutation). Further, the BAF and log R can be used to obtain corresponding major and minor copy numbers. In embodiments, the values of copy number metrics may be provided for both a tumour copy number profile estimate and a normal copy number profile estimate, even if only the tumour copy number profile values are used.

The terms “tumour-specific mutation”, “somatic mutation” or simply “mutation” are used interchangeably and refer to a difference in a nucleotide sequence (e.g. DNA or RNA) in a tumour cell compared to a healthy cell from the same subject. The difference in the nucleotide sequence can result in the expression of a protein which is not expressed by a healthy cell from the same subject. For example, a mutation may be a single nucleotide variant (SNV), multiple nucleotide variant (MNV), a deletion mutation, an insertion mutation, a translocation, a missense mutation, a translocation, a fusion, a splice site mutation, or any other change in the genetic material of a tumour cell. A mutation may result in the expression of a protein or peptide that is not present in a healthy cell from the same subject. Mutations may be identified by exome sequencing, RNA-sequencing, whole genome sequencing and/or targeted gene panel sequencing and or routine Sanger sequencing of single genes, followed by sequence alignment and comparing the DNA and/or RNA sequence from a tumour sample to DNA and/or RNA from a reference sample or reference sequence (e.g. the germline DNA and/or RNA sequence, or a reference sequence from a database). Suitable methods are known in the art.

An “indel mutation” refers to an insertion and/or deletion of bases in a nucleotide sequence (e.g. DNA or RNA) of an organism. Typically, the indel mutation occurs in the DNA, preferably the genomic DNA, of an organism. In embodiments, the indel may be from 1 to 100 bases, for example 1 to 90, 1 to 50, 1 to 23 or 1 to 10 bases. An indel mutation may be a frameshift indel mutation. A frameshift indel mutation is a change in the reading frame of the nucleotide sequence caused by an insertion or deletion of one or more nucleotides. Such frameshift indel mutations may generate a novel open-reading frame which is typically highly distinct from the polypeptide encoded by the non-mutated DNA/RNA in a corresponding healthy cell in the subject.

A “neoantigen” (or “neo-antigen”) is an antigen that arises as a consequence of a mutation within a cancer cell. Thus, a neoantigen is not expressed (or expressed at a significantly lower level) by normal (i.e. non-tumour) cells. A neoantigen may be processed to generate distinct peptides which can be recognised by T cells when presented in the context of MHC molecules. As described herein, neoantigens may be used as the basis for cancer immunotherapies. References herein to “neoantigens” are intended to include also peptides derived from neoantigens. The term “neoantigen” as used herein is intended to encompass any part of a neoantigen that is immunogenic. An “antigenic” molecule as referred to herein is a molecule which itself, or a part thereof, is capable of stimulating an immune response, when presented to the immune system or immune cells in an appropriate manner. The binding of a neoantigen to a particular MHC molecule (encoded by a particular HLA allele) may be predicted using methods which are known in the art. Examples of methods for predicting MHC binding include those described by Lundegaard et al., O'Donnel et al., and Bullik-Sullivan et al. For example, MHC binding of neoantigens may be predicted using the netMHC-3 (Lundegaard et al.) and netMHCpan4 (Jurtz et al.) algorithms. A neoantigen that has been predicted to bind to a particular MHC molecule is thereby predicted to be presented by said MHC molecule on the cell surface.

A “clonal neoantigen” (also sometimes referred to as “truncal neoantigen”) is a neoantigen that results from a mutation that is present in essentially every tumour cell in one or more samples from a subject (or that can be assumed to be present in essentially every tumour cell from which the tumour genetic material in the sample(s) is derived). Similarly, a “clonal mutation” (sometimes referred to as “truncal mutation”) is a mutation that is present in essentially every tumour cell in one or more samples from a subject (or that can be assumed to be present in essentially every tumour cell from which the tumour genetic material in the sample(s) is derived). Thus, a clonal mutation may be a mutation that is present in every tumour cell in one or more samples from a subject. A “sub-clonal” neoantigen is a neoantigen that results from a mutation that is present in a subset or a proportion of cells in one or more tumour samples from a subject (or that can be assumed to be present in a subset of the tumour cells from which the tumour genetic material in the sample(s) is derived). Similarly, a “sub-clonal” mutation is a mutation that is present in a subset or a proportion of cells in one or more tumour samples from a subject (or that can be assumed to be present in a subset of the tumour cells from which the tumour genetic material in the sample(s) is derived). A neoantigen or mutation may be clonal in the context of one or more samples from a subject while not being truly clonal in the context of the entirety of the population of tumour cells that may be present in a subject (e.g. including all regions of a primary tumour and metastasis). Thus, a clonal mutation may be “truly clonal” in the sense that it is a mutation that is present in essentially every tumour cell (i.e. in all tumour cells) in the subject. This is because the one or more samples may not be representative of each and every subset of cells present in the subject. Thus, within the context of the present disclosure, a “clonal neoantigen” or “clonal mutation” may also be referred to as a “ubiquitous neoantigen” or “ubiquitous mutation”, to indicate that the neoantigen is present in essentially all tumour cells that have been analysed, but may not be present in all tumour cells that may exist in the subject. The terms “clonal” and “ubiquitous” are used interchangeably unless context indicates that reference to “true clonality” was intended. The wording “essentially every tumour cell” in relation to one or more samples or a subject may refer to at least 70%, at least 75%, at least 80%, at least 85%, at least 90%, at least 91%, at least 92%, at least 93%, at least 94% at least 95%, at least 96%, at least 97%, at least 98%, or at least 99% of the tumour cells in the one or more samples or the subject.

Nevertheless, a neoantigen/mutation that is identified as likely to be clonal (or “ubiquitous”) as described herein is likely to be truly clonal, or at least more likely to be truly clonal than a neoantigen/mutation that is identified as unlikely to be clonal. Further, the confidence in the likelihood that a clonal neoantigen/mutation identified herein is truly clonal increases when the sample(s) used to identify the clonal neoantigen/mutation capture a more complete picture of the genetic diversity of the tumour (e.g. by including a plurality of samples from the subject, such as e.g. samples from different regions of the tumour, and/or by including samples that inherently capture a diversity of tumour cells such as e.g. ctDNA samples). Conversely, a neoantigen/mutation that is identified as unlikely to be clonal as described herein is unlikely to be truly clonal, because the identification that the neoantigen/mutation is unlikely to be clonal indicates that even in the restricted view afforded by the sampling process, there is evidence that the neoantigen/mutation is not present in all tumour cells. Thus, the process of identifying clonal neoantigens/mutations may be seen as prioritising which candidate neoantigens/mutations are most likely to be clonal, based on the restricted view of the clonal structure of the subject's tumour available from the one or more samples.

The term “cancer cell fraction” (or “CCF”) refers to the proportion of tumour cells that contain a mutation, such as e.g. a mutation that results in a particular neoantigen. Within the context of the present disclosure, the cancer cell fraction may be estimated based on one or more samples, and as such may not be equal to the true cancer cell fraction in the subject (as explained above). Nevertheless, the cancer cell fraction estimated based on one or more samples may provide a useful indication of the likely true cancer cell fraction. Further, as explained above, the accuracy of such an estimate may increase when the sample(s) used to estimate the cancer cell fraction capture a more complete picture of the genetic diversity of the tumour. Additional sources of noise and confounding factors in genomic data mean that a cancer cell fraction determined from one or more samples represents an estimate. As such, although a truly clonal mutation/neoantigen should have a CCF=1, in practice mutations/neoantigens that are more likely to be clonal are expected to be associated with a higher CCF estimate (which may not be equal to 1) than mutations that are less likely to be clonal, which are expected to be associated with a lower CCF estimate.

For example, a cancer cell fraction estimate may be obtained by integrating variant allele frequencies with copy numbers and purity estimates as described by Landau et al. (2013). Such a CCF estimate can also be used to identify mutations that are likely to be clonal. For example, a clonal mutation may be defined as a mutation which has an estimated cancer cell fraction (CCF)≥0.75, such as a CCF≥0.80, 0.85, 0.90, 0.95 or 1.0. A subclonal mutation may be defined as a mutation which has a CCF<0.95, 0.90, 0.85, 0.80, or 0.75. Further, a CCF estimate may be associated with (e.g. derived from) a distribution associating a probability with each of a plurality of possible values of CCF between 0 and 1, from which statistical estimates of confidence may be obtained. For example, a mutation may be defined as likely to be a clonal mutation if the 95% CCF confidence interval is >=0.75, i.e. the upper bound of the 95% confidence interval of the estimated CCF is greater than or equal to 0.75. In other words, a mutation may be defined as likely to be a clonal mutation if there is an interval of CCF with lower bound L and upper bound H that is such that P(L<CCF<H)=95% with H>=0.75. Alternatively, a mutation may be identified as clonal if there is more than a 50% chance or probability that its cancer cell fraction (CCF) reaches or exceeds the required value as defined above, for example 0.75 or 0.95, such as a chance or probability of 50%, 55%, 60%, 65%, 70%, 75%, 80%, 85%, 90%, 95% or more. In other words, a mutation may be identified as clonal if P(CCF>0.75)>=0.5. For example, mutations may be classified as likely clonal or subclonal based on whether the posterior probability that their CCF exceeds 0.95 (or 0.75, or any other chosen threshold) is greater or lesser than 0.5, respectively.

According to the methods of the present disclosure, as will be described further below, a likelihood of a mutation being clonal is obtained. This is equivalent to P(CCF=1). In this context, as will be explained further below, a mutation may be identified as likely to be clonal if P(CCF=1) exceeds a threshold. The threshold may be fixed. For example, a mutation may be identified as likely to be clonal if P(CCF=1)>0.05. Alternatively, the threshold may be determined for a particular set of mutations that are investigated. In embodiments, the threshold may be set based on a benchmarking data set with known clonal/non-clonal status, to reach a predetermined precision and/or recall. A benchmarking data set may be obtained using synthetic data and/or using a data set obtained from a population with known clonality structure (for example a cell line mixture data). For example, a mutation may be identified as likely clonal if P(CCF=1)>t where t is the maximum value that is such that 95% (or any other value such as e.g. 50%, 55%, 60%, 65%, 70%, 75%, 80%, 85%, 90%) of true clonal mutations in a benchmarking dataset are identified (i.e. a false negative rate of at most 5%). As another example, a mutation may be identified as likely clonal if P(CCF=1)>t where t is the minimum value that is such that at least 50% (or any other value such as e.g. 50%, 55%, 60%, 65%, 70%, 75%, 80%, 85%, 90%) of the mutations that exceed the threshold in a benchmarking dataset are true clonal mutations (i.e. a true positive rate of at least 50%). Alternatively, the threshold may be set such that any mutation (or a certain % of mutations) that is associated with an estimated CCF that has a confidence interval meeting the criteria described above (e.g.it is such that the upper bound of the 95% confidence interval of the estimated CCF is greater than or equal to 0.75) is selected as likely to be clonal. Alternatively, the threshold may be set such that any mutation (or a certain % of mutations) that is associated with an estimated CCF that has a posterior probability distribution meeting the criteria described above (e.g. a posterior probability that their CCF exceeds 0.95 (or 0.75, or any other chosen threshold) is greater than 0.5) is selected as likely to be clonal.

A cancer immunotherapy (or simply “immunotherapy”) refers to a therapeutic approach comprising administration of an immunogenic composition (e.g. a vaccine), a composition comprising immune cells, or an immunoactive drug, such as e.g. a therapeutic antibody, to a subject. The term “immunotherapy” may also refer to the therapeutic compositions themselves. In the context of the present disclosure, the immunotherapy typically targets a neoantigen. For example, an immunogenic composition or vaccine may comprise a neoantigen, neoantigen presenting cell or material necessary for the expression of the neoantigen. As another example, a composition comprising immune cells may comprise T and/or B cells that recognise a neoantigen. The immune cells may be isolated from tumours or other tissues (including but not limited to lymph node, blood or ascites), expanded ex vivo or in vitro and re-administered to a subject (a process referred to as “adoptive cell therapy”). Instead or in addition to this, T cells can be isolated from a subject and engineered to target a neoantigen (e.g. by insertion of a chimeric antigen receptor that binds to the neoantigen) and re-administered to the subject. As another example, a therapeutic antibody may be an antibody which recognises a neoantigen. One skilled in the art will appreciate that if the neoantigen is a cell surface antigen, an antibody as referred to herein will recognise the neoantigen. Where the neoantigen is an intracellular antigen, the antibody will recognise the neoantigen peptide-MHC complex. As referred to herein, an antibody which “recognises” a neoantigen encompasses both of these possibilities. Further, an immunotherapy may target a plurality of neoantigens. For example, an immunogenic composition may comprise a plurality of neoantigens, cells presenting a plurality of neoantigens or the material necessary for the expression of the plurality of neoantigens. As another example, a composition may comprise immune cells that recognise a plurality of neoantigens. Similarly, a composition may comprise a plurality of immune cells that recognise the same neoantigen. As another example, a composition may comprise a plurality of therapeutic antibodies that recognise a plurality of neoantigens. Similarly, a composition may comprise a plurality of therapeutic antibodies that recognise the same neoantigen.

A composition as described herein may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

References to “an immune cell” are intended to encompass cells of the immune system, for example T cells, NK cells, NKT cells, B cells and dendritic cells. In a preferred embodiment, the immune cell is a T cell. An immune cell that recognises a neoantigen may be an engineered T cell. A neoantigen specific T cell may express a chimeric antigen receptor (CAR) or a T cell receptor (TCR) which specifically binds a neoantigen or a neoantigen peptide, or an affinity-enhanced T cell receptor (TCR) which specifically binds a neoantigen or a neoantigen peptide (as discussed further hereinbelow). For example, the T cell may express a chimeric antigen receptor (CAR) or a T cell receptor (TCR) which specifically binds to a neo-antigen or a neo-antigen peptide (for example an affinity enhanced T cell receptor (TCR) which specifically binds to a neo-antigen or a neo-antigen peptide). Alternatively, a population of immune cells that recognise a neoantigen may be a population of T cell isolated from a subject with a tumour. For example, the T cell population may be generated from T cells in a sample isolated from the subject, such as e.g. a tumour sample, a peripheral blood sample or a sample from other tissues of the subject. The T cell population may be generated from a sample from the tumour in which the neoantigen is identified. In other words, the T cell population may be isolated from a sample derived from the tumour of a patient to be treated, where the neoantigen was also identified from a sample from said tumour. The T cell population may comprise tumour infiltrating lymphocytes (TIL).

The term “Antibody” (Ab) includes monoclonal antibodies, polyclonal antibodies, multispecific antibodies (e.g., bispecific antibodies), and antibody fragments that exhibit the desired biological activity. The term “immunoglobulin” (Ig) may be used interchangeably with “antibody”. Once a suitable neoantigen has been identified, for example by a method according to the disclosure, methods known in the art can be used to generate an antibody.

An “immunogenic composition” is a composition that is capable of inducing an immune response in a subject. The term is used interchangeably with the term “vaccine”. The immunogenic composition or vaccine described herein may lead to generation of an immune response in the subject. An “immune response” which may be generated may be humoral and/or cell-mediated immunity, for example the stimulation of antibody production, or the stimulation of cytotoxic or killer cells, which may recognise and destroy (or otherwise eliminate) cells expressing antigens corresponding to the antigens in the vaccine on their surface. The immunogenic composition may comprise one or more neoantigens, or the material necessary for the expression of one or more neoantigens. In addition, a neoantigen may be delivered in the form of a cell, such as an antigen presenting cell, for example a dendritic cell. The antigen presenting cell such as a dendritic cell may be pulsed or loaded with the neo-antigen or neo-antigen peptide or genetically modified (via DNA or RNA transfer) to express one, two or more neo-antigens or neoantigen peptides, for example 2, 3, 4, 5, 6, 7, 8, 9 or 10 neo-antigens or neo-antigen peptides. Methods of preparing dendritic cell immunogenic compositions or vaccines are known in the art.

Neoantigen peptides may be synthesised using methods which are known in the art. The term “peptide” is used in the normal sense to mean a series of residues, typically L-amino acids, connected one to the other typically by peptide bonds between the a-amino and carboxyl groups of adjacent amino acids. The term includes modified peptides and synthetic peptide analogues. The neoantigen peptide may comprise the cancer cell specific mutation (e.g the non-silent amino acid substitution encoded by a single nucleotide variant (SNV)) at any residue position within the peptide. By way of example, a peptide which is capable of binding to an MHC class I molecule is typically 7 to 13 amino acids in length. As such, the amino acid substitution may be present at position 1, 2, 3, 4, 5, 6, 7, 8, 9, 10, 11, 12 or 13 in a peptide comprising thirteen amino acids. In embodiments, longer peptides, for example 21-31-mers, may be used, and the mutation may be at any position, for example at the centre of the peptide, e.g. at positions 10, 11, 12, 13, 14, 15 or 16. Such peptides can also be used to stimulate both CD4 and CD8 cells to recognise neoantigens.

As used herein “treatment” refers to reducing, alleviating or eliminating one or more symptoms of the disease which is being treated, relative to the symptoms prior to treatment. “Prevention” (or prophylaxis) refers to delaying or preventing the onset of the symptoms of the disease. Prevention may be absolute (such that no disease occurs) or may be effective only in some individuals or for a limited amount of time.

As used herein, the terms “computer system” includes the hardware, software and data storage devices for embodying a system or carrying out a method according to the above described embodiments. For example, a computer system may comprise a central processing unit (CPU), input means, output means and data storage, which may be embodied as one or more connected computing devices. Preferably the computer system has a display or comprises a computing device that has a display to provide a visual output display (for example in the design of the business process). The data storage may comprise RAM, disk drives or other non-transitory computer readable media. The computer system may include a plurality of computing devices connected by a network and able to communicate with each other over that network. It is explicitly envisaged that computer system may consist of or comprise a cloud computer.

As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### Identification of Clonal Mutations

The present disclosure provides methods for determining whether a tumour-specific mutation is likely to be clonal using sequence data from one or more samples comprising tumour cells or genetic material derived therefrom, and an indication of whether the mutation is likely to be associated with one or more mutational signatures. The disclosure also provides methods for identifying clonal neoantigens comprising determining whether one or more tumour-specific mutations is/are likely to be clonal.

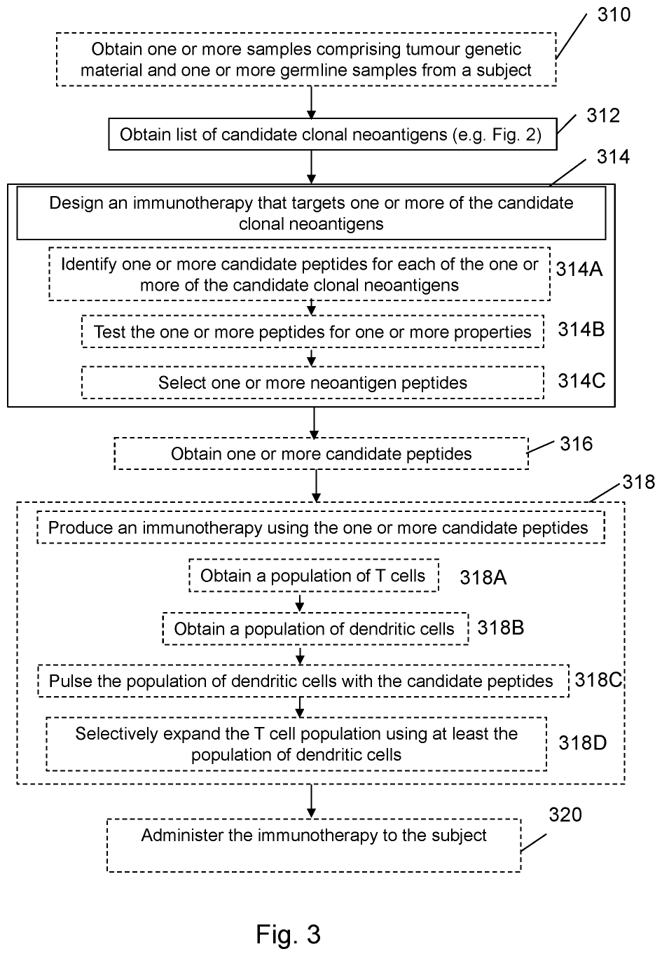

An illustrative method will be described by reference to FIGS. 2 and 3. In particular, FIG. 2 illustrates an example of a method for determining the prior probability of a plurality of tumour-specific mutations being clonal according to a method described herein. FIG. 3 illustrates an embodiment where a plurality of tumour specific mutations are identified and assessed for clonality. However, the method is applicable to any number of mutations, including a single mutation. FIG. 3 illustrates an example of a method for determining whether a tumour-specific mutation is likely to be clonal using a prior probability obtained using a method as described herein. As illustrated on FIG. 2, at optional step 110, one or more samples comprising genomic material from a tumour may be obtained from a subject. A matched sample that does not comprise genomic material from tumour cells, or from which genomic material from normal cells can be extracted, may also be obtained or may have been previously obtained. At optional step 112, the sequence content of the one or more samples may be determined, for example by sequencing the genomic material in the sample using one of whole exome sequencing, or whole genome sequencing. At optional step 114, the sequence data may be analysed to obtain a mutational profile for each of the samples comprising genomic material from the tumour. This may comprise identifying mutations that are likely to be present in the tumour cells but not in non-cancerous cells (for example using both the tumour sample(s) and the matched sample), also referred to as somatic mutations or tumour-specific mutations, and classifying each somatic mutation in one of a plurality of predetermined mutational classes. Alternatively, the method may start from one or more mutational profiles and sequence data associated with the tumour sample(s). The sequence data may comprise sequencing reads. The sequencing reads may comprise all reads obtained by whole exome, whole genome or targeted sequencing of the samples, or a subset thereof. The sequence data may comprise for each tumour-specific mutation, a count of reads supporting the mutated allele (also referred to as “non-reference allele”), a count of reads supporting the germline allele(s) (A, collectively referred to as “germline allele” if the locus is heterozygous in the germline population, also referred to as “reference”, “wild type” or “normal” allele) at the genomic location, and/or the total count of reads at the genomic location of each tumour-specific mutation. At step 116, an indication of whether each mutation is associated with one or more predetermined mutational signatures is obtained. This may comprise determining the exposure of each mutational profile to each of the predetermined mutational signatures, and determining for each mutation a set of mutation-specific weights for the predetermined mutational signatures. At step 118, a metric indicative of the probability that each mutation is clonal is obtained by combining evidence form the sequence data obtained at step 112 and the metric obtained at step 116. The metric indicative of the probability that a mutation is clonal may be a posterior probability. For example, the metric may be a posterior probability that the mutation is clonal. A method to estimate this is described below and by reference to FIG. 3. As another example, the posterior probability may be a posterior probability of one or more cancer cell fractions for the mutation. Methods for determining such posterior probabilities are described in Landau et al. 2013, and WO 2016/16174085. Thus, determining a metric indicative of the probability that a mutation is clonal at step 118 may comprise determining a posterior probability based on: a prior probability of the mutation being clonal that depends on the indication of whether the mutation is associated with the one or more predetermined mutational signatures determined at step 116, and a probability of observing the sequence data if the mutation is clonal, non-clonal and/or has a particular cancer cell fraction (i.e. likelihood of clonality in view of data) determined e.g. as described below or in Landau et al. 2013, WO 2016/16174085, or using other Bayesian methods in the art. The prior probability of a mutation being clonal may be obtained as the output of a model that predicts the prior probability of the mutation being clonal using inputs comprising the indication of whether the mutation is associated with each of the mutational signatures. The model may a linear model, a logistic regression model or a simple linear model. The model may have been trained or fitted using training data obtained at optional steps 120 and 122 by obtaining mutational profiles for a plurality of training samples and associated clonal status, and obtaining from these mutational profiles, indications of whether the mutations are associated with each of the mutational signatures. These may be used to fit a model at step 124 that predicts the probability of a mutation being clonal (or the log odds of a mutation being clonal) as a function of the indication of association between mutations and signatures. The training may comprise identifying coefficients of the model that best predict the known clonal status for each of the mutations in the training data, based on the indications of associations between mutations and signatures (i.e. mutation-specific weights for each of the signatures). Alternatively, the training may comprise identifying the proportion of mutations assigned to each signature in the training data that are clonal, where mutations may be assigned to a signature using the mutation-specific weights for each of the signatures, for example each mutation in the training data being assigned to the signature with highest weight for that mutation.

Turning now to FIG. 3, at optional step 210, a sample comprising genomic material from a tumour may be obtained from a subject. The sample is typically a mixed sample comprising genomic material from multiple cell types including tumour cells. Preferably, a matched sample that does not comprise genomic material from tumour cells, or from which genomic material from normal cells can be extracted, may be obtained or may have been previously obtained. A matched sample is a sample obtained from the same subject as the tumour sample. The use of a matched normal sample improves the accuracy of calling of somatic (tumour-specific) mutations, as any variant position identified in the tumour sample can be compared to variant positions in the matched normal sample to exclude germline variants. The same matched normal sample may be used to analyse a plurality of tumour samples from a subject. Further, the matched sample and one or more tumour samples may have been obtained at different times. For example, a first tumour sample and matched sample may have been obtained at the time of diagnosis or resection of a tumour, and a further tumour sample may be obtained and analysed together with the initial matched sample at a later time point. When a matched sample is not available, a reference sample or genome including common somatic variants may be used. Alternatively, a processed matched normal sample may be used, which may not have been obtained from the same subject, or may have been obtained from a pool of subjects.

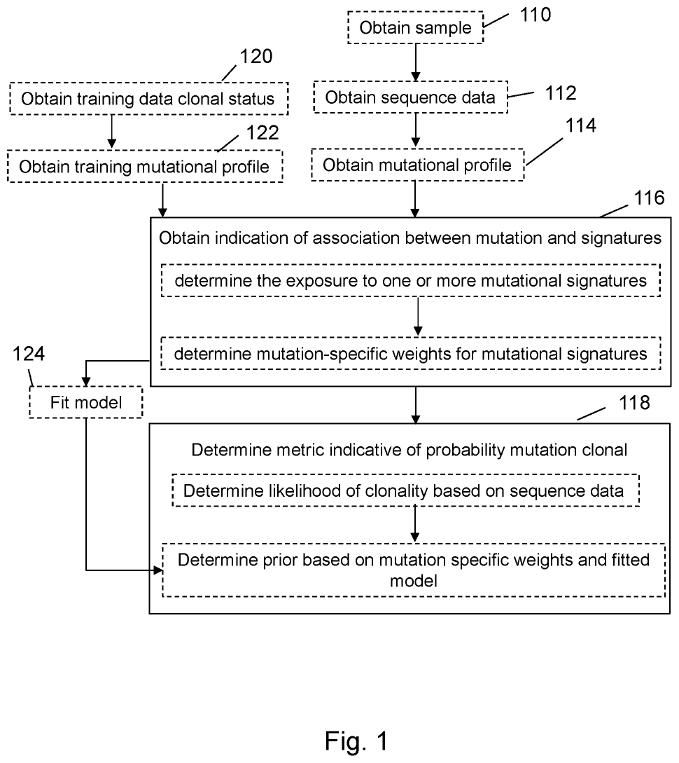

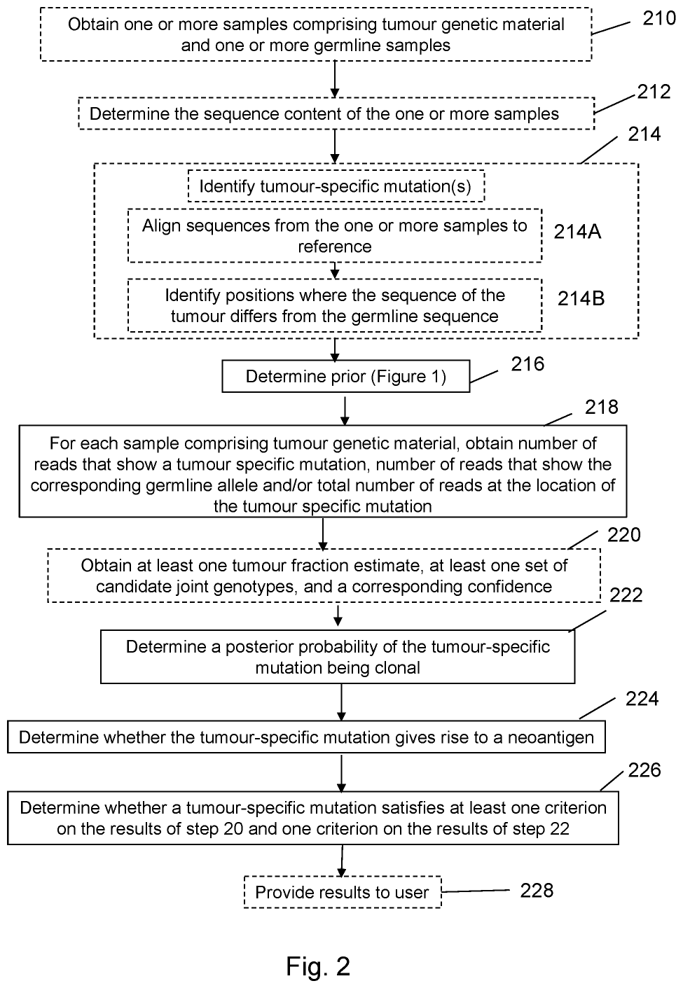

At optional step 212, the sequence content of the one or more mixed samples and optionally the matched sample may be determined, for example by sequencing the genomic material in the sample using one of whole exome sequencing, or whole genome sequencing. Alternative methods such as e.g. allele-specific copy number arrays may be used, although sequencing methods are preferred since they generate a digital output representative of the number of each particular sequence in a sample. At optional step 214, the sequence data may be analysed to identify one or more mutations that are likely to be present in the tumour cells but not in non-cancerous cells. These represent tumour-specific mutations and may be used as candidate neoantigens. This may comprise the steps of aligning the sequences from the one or more samples (i.e. the mixed sample(s) and the germline sample(s), if available), and identifying genomic locations where the sequence of the tumour differs from the germline sequence or can be assumed to differ from the germline sequence (e.g. if a germline sequence for the subject is not available). Each of steps 210-214 may have been previously performed and the method may start from a list of mutations and the sequence data used at step 218. For example, steps 210-214 may have been performed as part of a method as described by reference to FIG. 1, or may have been performed prior to commencing any method described herein. At step 216, a prior probability of a mutation being clonal is obtained for each mutation based on an indication of whether the mutation is associated with one or more predetermined mutational signatures, using a method as described herein such as that described by reference to FIG. 1.

At step 218, sequence data for the mixed sample at the genomic location of a candidate tumour-specific mutation is obtained, comprising the count of reads supporting the mutated allele (also referred to as “non-reference allele”), the count of reads supporting the germline allele(s) (A, collectively referred to as “germline allele” if the locus is heterozygous in the germline population, also referred to as “reference”, “wild type” or “normal” allele) at the genomic location, and/or the total count of reads at the genomic location of the candidate tumour-specific mutation. Only two of these metrics need to be obtained as the third one can be deduced from any two of these. The sequence data may instead or in addition to this include read data or intensity data from which the counts can be obtained. At optional step 220, information about at least one copy number solution compatible with each sample comprising tumour-genetic material may be obtained. This information may comprise allele-specific copy number metrics for the tumour fraction of the sample selected from the major copy number, minor copy number, total copy number, mean B allele frequency, log R value and tumour ploidy, and the normal copy number, or information derived from these metrics such as a set of candidate joint genotypes that is compatible with these allele-specific copy number metrics. Not all such allele-specific copy number metrics are necessary as some contain redundant information and/or can be associated with suitable default values. For example, the normal copy number can be associated with a suitable default value as explained above. Further, only two of the major copy number, total copy number and minor copy number are necessary to infer the third one. Similarly, those three values can be inferred from the MBAF and log R values (and vice versa). Optionally, a copy number solution may be associated with a corresponding confidence metric. When such a metric is not available, each copy number solution may be assumed to be equally likely. Each candidate joint genotype comprises a genotype at the location of the tumour-specific mutation for a normal population, a reference tumour population that does not comprise the tumour-specific mutation and a variant tumour cell population that comprises the tumour-specific mutation.

At step 222, the probability of a tumour-specific mutation being clonal is determined as a posterior probability depending on the evidence from sequence data obtained at step 218 and the prior probability obtained at step 216. At step 224, it is determined whether the tumour-specific mutation is likely to give rise to a neoantigen. For example, it may be determined whether the mutation is likely to result in a peptide or protein that is not expressed by a germline cell (whose genome does not contain the mutation). This step may be performed at any point after step 214, and in particular need not be performed after steps 216-222. For example, candidate tumour-specific mutations may be filtered depending on whether they are likely to give rise to a neoantigen prior to determining whether the tumour-specific mutation is likely to be clonal. At step 226, tumour-specific mutations that satisfy one or more criteria that apply to the results of step 222 and one or more criteria that apply to the results of step 224 may be identified. These may be considered to represent candidate clonal neoantigens. At optional step 228, the results of any of the preceding steps (and in particular steps 222 to 226) may be provided to a user, for example through a user interface. These results may be used for example to provide an immunotherapy or prognosis for a subject, as will be described further below.

A clonal neoantigen may be a tumour-specific mutation that satisfies at least a criterion selected from: having a probability of being clonal above a predetermined threshold, having a probability of being clonal that is above a threshold set adaptively to select a predetermined number of tumour-specific mutations with the highest probabilities of being clonal amongst the tumour-specific mutations for which a probability was determined, and having a probability of being clonal that is above a threshold set adaptively to select a predetermined top percentile of tumour-specific mutations amongst the tumour-specific mutations for which a probability was determined. Thus, the one or more predetermined criteria on whether the tumour-specific mutation is likely to be clonal may be selected from: the mutation having a likelihood of being clonal above a predetermined threshold, the mutation having a likelihood of being clonal that is above a threshold set adaptively to select a predetermined number of tumour-specific mutations with the highest likelihoods of being clonal amongst the tumour-specific mutations for which a likelihood was determined, and having a likelihood of being clonal that is above a threshold set adaptively to select a predetermined top percentile of tumour-specific mutations amongst the tumour-specific mutations for which a likelihood was determined.

A clonal neoantigen may be a tumour-specific mutation that satisfies at least a criterion selected from: being associated with an expression product that is expressed in tumour cells, being predicted to result in a protein or peptide that is not expressed in the normal cells of the subject, being predicted to result in at least one peptide that is likely to be presented by an MHC molecule, being predicted to result in at least one peptide that is likely to be presented by an MHC allele that is known to be present in the subject, and being predicted to result in a protein or peptide that is immunogenic. For example, a clonal neoantigen may be a tumour-specific mutation that satisfies a criterion that it is predicted to result in a change in the sequence of a protein (e.g. because it is coding, because it affects a splice site, because it results in a truncated peptide, etc.), thus resulting in a protein or peptide that may not be expressed in the normal cells of the subject. Whether or not this is the case may further be confirmed for example by comparison with a predicted normal proteome of the subject. Thus, the one or more criteria on whether the tumour-specific mutation is likely to give rise to a neoantigen may be selected from: the mutation being associated with an expression product that is expressed in tumour cells, the mutation being predicted to result in a protein or peptide that is not expressed in the normal cells of the subject, the mutation being predicted to result in at least one peptide that is likely to be presented by an MHC molecule, the mutation being predicted to result in at least one peptide that is likely to be presented by an MHC allele that is known to be present in the subject, and the mutation being predicted to result in a protein or peptide that is immunogenic.

The method may further comprise identifying one or more peptides associated with the one or more clonal neoantigens (i.e. one or more peptide sequences that are predicted to be present in the tumour cells as a consequence of the presence of the tumour-specific mutation, where the tumour-specific mutation satisfies one or more criteria (related to likelihood of clonality and likelihood of giving rise to a clonal neoantigen) as described above.

As the skilled person understands, the complexity of the operations described herein (due at least to the complexity of obtaining posterior probabilities requiring numerical integration as described herein, and the amount of data that is typically generated by sequencing genomic DNA) are such that they are beyond the reach of a mental activity. Thus, unless context indicates otherwise (e.g. where sample preparation or acquisition steps are described), all steps of the methods described herein are computer implemented.

At step 222, the posterior probability of a tumour-specific mutation being clonal may be determined as a posterior probability depending on: the prior probability of the mutation being clonal determined at step 216, and the probabilities of observing the sequence data if the tumour-specific mutation is (i) clonal and (ii) non-clonal, in view of a tumour fraction for each of the one or more samples and one or more candidate joint genotypes each comprising a genotype at the location of the tumour-specific mutation for a normal population, a reference tumour population that does not comprise the tumour-specific mutation and a variant tumour cell population that comprises the tumour-specific mutation. Such a method obtains a probability that a mutation is clonal (P(Z=1)) as a posterior probability (p(Z=1|d_{b},d,π,t,ρ)) that depends on the prior probability of the mutation being clonal (p), and the probability of observing the sequence data (also referred to as the “likelihood” of observing the sequence data, or simply the “likelihood” of the sequence data). An example of such a method id described in the Examples section. Such a method may have one or more of the following features. Thus, the step of obtaining the sequence data may be performed by a processor, and the step of determining the likelihood that the tumour-specific mutation is clonal may be performed by said processor. The step of obtaining the sequence data may comprise receiving sequence data comprising sequence reads from one or more samples from the subject, and determining from said sequence reads at least two of: the number of reads in the sample that show the tumour-specific mutation (d_{b}), the number of reads in the sample that show the corresponding germline allele, and the total number of reads at the location of the tumour-specific mutation (d). At least the step of determining the likelihood that the tumour-specific mutation is clonal may be computer implemented. The step of determining the likelihood that the tumour-specific mutation is clonal may comprise a step of numerical integration to obtain the posterior probability. In particular, the step may comprise determining the posterior probability that the mutation is clonal in view of a prior probability of the mutation being clonal, and the probabilities of observing the sequence data if the tumour-specific mutation is (i) clonal and (ii) non-clonal, by solving a plurality of one dimensional integrals (such as e.g. a pair of integrals for each sample, respectively representing the assumption that the mutation is clonal and non-clonal) integrating the probability of the observed sequence data over all possible cancer cell fractions between 0 and 1. These numerical integrals may be solved independently (such as e.g. in parallel) for each sample and each mutation.

The probability that the tumour-specific mutation is clonal may depend on the prior probability of the mutation being clonal (ρ) through: a prior probability of the mutation being assigned to a clonal category given the prior probability of the mutation being clonal (P(Z=1|ρ)=ρ); and a prior probability of the mutation being assigned to a non-clonal category given the prior probability of the mutation being clonal (P(Z=0|ρ)=(1−ρ)). The prior probability of the mutation being clonal is the probability determined at step 216, according to the methods of the present disclosure. The probability of observing the sequence data if the tumour-specific mutation is clonal (in view of a tumour fraction for each of the one or more samples and one or more candidate joint genotypes) may be marginalised over the cancer cell fraction. Similarly, the probability of observing the sequence data if the tumour-specific mutation is not clonal, in view of a tumour fraction for each of the one or more samples and one or more candidate joint genotypes, may be marginalised over the cancer cell fraction.

The probability that the tumour-specific mutation is clonal may depend on: the prior probability of the mutation being assigned to a clonal category given the prior probability of the mutation being clonal (P(Z=1|ρ)=ρ), multiplied by the probability in each sample of observing the sequence data in view of a tumour fraction, and one or more candidate joint genotypes, if the mutation is clonal (which can be calculated as L₁, the likelihood of the sequence data in each sample, marginalised over the cancer cell fraction); and the prior probability of the mutation being assigned to a non-clonal category given the prior probability of the mutation being subclonal (P(Z=0|ρ)=1−ρ), multiplied by the probability in each sample of observing the sequence data in view of a tumour fraction and one or more candidate joint genotypes, if the mutation is non-clonal (which can be calculated as ψ₀, the likelihood of the sequence data in each sample, marginalised over the cancer cell fraction).

The probability that the tumour specific mutation is clonal may be obtained as the ratio of (i) the prior probability of the mutation being assigned to a clonal category given the prior probability of the mutation being clonal multiplied by the probability of observing the sequence data in each sample in view of a tumour fraction and one or more candidate joint genotypes, if the mutation is clonal (p(d_{b}, d, Z=1|π,t, ρ), which can be expressed as ρψ₁), divided by (ii) the sum of (i) (i.e. p(d_{b}, d, Z=1|π,t, ρ)) and the prior probability of the mutation being assigned to a non-clonal category given the prior probability of the mutation being subclonal, multiplied by the probability of observing the sequence data in each sample view of a tumour fraction in each sample and one or more candidate joint genotypes, if the mutation is non-clonal (p(d_{b}, d, Z=0|π,t, ρ), which can be expressed as (1−ρ)ψ₀).

The probability that a mutation is clonal may be obtained using equation (11a). In equation (11a), the term Pr(d_{b},d|π,ϕ,t) may be given by any of equations (3), (4), (3a), (4a), (3b) or (4b). In equation (11), the terms p(ϕ|Z=0) and p(ϕ|z=1) may be given by equation (6).

A clonal mutation may be a mutation that is present in all or essentially all tumour cells in the one or more samples from the subject comprising tumour genetic material (or in all of the tumour genetic material in the one or more samples). Such a mutation may be, or may be assumed to be (as full certainty on this may be associated with sequencing of all tumour cells in the subject, but presence in essentially all cells in one or more samples may be used as an indication of this), present in all tumour cells in the subject.

The probability of observing the sequence data in view of a tumour fraction in each sample and one or more candidate joint genotypes may depend on the probability of observing the sequence data in view of a tumour fraction, cancer cell fraction and one or more candidate joint genotypes (Pr(d, db|π, ϕ, t)). The probability of observing the sequence data in view of a tumour fraction, cancer cell fraction and one or more candidate joint genotypes may be a weighted sum of the probabilities of observing the sequence data in view of a tumour fraction, cancer cell fraction and each of the one or more candidate joint genotypes.

Advantageously, the probability of observing the sequence data (likelihood of the sequence data) may be calculated over a plurality of candidate genotypes (e.g. as a sum of probabilities comprising a term for each candidate genotype, see e.g. equations (3a), (3b)), the contribution of which may be weighted for example to reflect prior knowledge on the relative probabilities of the candidate genotypes (e.g. any prior knowledge on whether some genotypes are more likely to occur than others). When no such prior knowledge is available or desirable, the probabilities for each of the candidate genotypes may be weighted equally. The weights of the respective candidate genotypes considered suitably sum to 1, such that the total probability reflects the relative contributions of the different candidate joint genotypes considered. When a single candidate joint genotype is used, it may be assigned a weight of 1 (i.e. no sum may be obtained).

The probability of observing the sequence data in view of a tumour fraction, cancer cell fraction and a particular candidate joint genotype (Gᵢ) (which can be calculated as ψ_{z}, the likelihood of the sequence data in each sample, marginalised over the cancer cell fraction) may be obtained using a Binomial distribution with parameters d_{b} and ξ(Gᵢ, ϕ, t). Alternatively, the probability of observing the sequence data in view of a tumour fraction, cancer cell fraction and a particular candidate joint genotype may be obtained using a BetaBinomial distribution with parameters d_{b}, ξ(Gᵢ, ϕ, t), and γ. In both cases (i.e. whether a Binomial or a BetaBinomial distribution is used), ξ(Gᵢ, ϕ, t) may represent the probability of sampling a read with the variant allele assuming a particular genotype Gi, a cancer cell fraction ϕ and a tumour purity t. The probability ξ(Gᵢ, ϕ, t) may be obtained as a function of the total number of copies for each of the normal, variant and reference genotypes, the probability of sampling a read with the variant from a population with genotype Gi in view of the proportion of alleles at the locus that are variant in the genotype and the sequencing error rate, the tumour fraction in the sample and the cancer cell fraction for the mutation.

The probability of observing the sequence data in view of a tumour fraction in each sample and one or more candidate joint genotypes may be obtained as an integral over all possible values of the cancer cell fraction in each sample, wherein the cancer cell fraction is the proportion of tumour cells that comprise the tumour-specific mutation. Thus, the step of determining the likelihood that the tumour-specific mutation is clonal may comprise using a processor to numerically integrate said integral.

The cancer cell fraction (ϕ) may take values between 0 and 1. In other words, the probability of observing the sequence data in view of a tumour fraction in each sample and one or more candidate joint genotypes, if the mutation is clonal or non-clonal may be obtained by integrating a value that is dependent on the cancer cell fraction over all possible values of the cancer cell fraction (i.e. marginalising over the cancer cell fraction). The value that is dependent on the cancer cell fraction may be expressed as Pr(d_{b}, d|π, ϕ, t) p(ϕ|Z=z) where the first term is the probability of observing the sequence data in view of a tumour fraction, cancer cell fraction and one or more candidate joint genotypes, and the second term is the prior probability (i.e. a probability based on assumptions of how the cancer cell fraction should behave for a clonal/non-clonal mutation) of a cancer cell fraction if the mutation is classified as clonal or non-clonal (Z=1 or Z=0, respectively). Thus, the probability of observing the sequence data in view of a tumour fraction in each sample and one or more candidate joint genotypes may be obtained as ∫₀¹ Pr(d_{b}, d|π, ϕ, t) p(ϕ|Z=z)dϕ.

The prior probability of a particular cancer cell fraction if the mutation is classified as clonal may be defined as a beta distribution with parameters α (set to a value>1, for example, 99, though any other value may be used) and ρ=1 (Beta(ϕ|α, 1)). The prior probability of a particular cancer cell fraction if the mutation is classified as non-clonal may be defined as a beta distribution with parameters α=1 and β=1 (Beta(ϕ|1,1)).

Sequence data from a plurality of samples may be obtained and the probability of observing the sequence data in view of a tumour fraction for each of the plurality of samples and one or more candidate joint genotypes may be obtained as the product of the probability of observing the sequence data of each sample in view of the tumour fraction in the respective sample and the one or more candidate joint genotypes. Thus, such a method is able to seamlessly integrate evidence for/against the clonality of a mutation obtained from multiple samples if these are available.

The method may further comprise obtaining or providing, for each sample, at least one estimate of the tumour fraction, and at least one corresponding set of one or more candidate joint genotypes. A tumour fraction estimate may be obtained using a method for determining allele-specific copy number profiles in samples comprising a mixture of tumour and normal cells. Methods for doing this using sequencing or array data are known in the art, for example by expressing the allele specific data as a function of parameters including allele-specific copy numbers, tumour aneuploidy and tumour cell fraction, and identifying the value of these parameters that best fit all of the data. Examples of such methods include e.g. ASCAT (Van Loo et al., 2010), amongst others. Alternatively, a tumour fraction estimate may be determined experimentally. Thus, the method may further comprise obtaining a tumour fraction estimate for each of the one or more samples. In particular, the method may comprise obtaining, by a processor, for each sample, at least one estimate of the tumour fraction comprises the processor determining an estimate of the tumour fraction and allele specific copy numbers using the sequence data, and determining, by said processor, a set of one or more candidate joint genotypes associated with said allele specific copy numbers.

A set of one or more candidate genotypes may be obtained using allele-specific copy numbers or variables derived therefrom (or conversely, from which such allele-specific copy numbers can be derived, such as B allele fraction and log R) for the tumour cells in a mixed sample. Allele-specific copy numbers for the tumour cells in a mixed sample may be obtained using a method for determining allele-specific copy number profiles in samples comprising a mixture of tumour and normal cells, such as e.g. ASCAT (Van Loo et al., 2010), or ascatNgs (Raine et al., 2016), amongst others.

Thus, the method may further comprise obtaining, for each of the one or more samples, estimates for at least two of: the copy number of the major allele in the tumour cells in the sample, the copy number of the minor allele in the tumour cells in the sample, and the total copy number at the location of the tumour-specific mutation in the tumour cells in the sample. The estimates of copy number in the tumour cells in the sample may represent a summarised (e.g. average) estimate over the entire population of tumour cells in the sample.

A set of one or more candidate joint genotypes may be obtained as the candidate joint genotypes that are compatible with the assumptions that: the normal population only comprises the normal allele(s) A (i.e. G_{H}=AA or A, e.g. if the locus is on a sex chromosome); the reference population does not comprise the variant allele B (i.e. G_{R}=(A)*n); and the variant population comprises at least one copy of the variant allele B (i.e. G_{V}=(A)*m(B)*I).

Advantageously, the set of candidate genotypes may comprise the candidate joint genotypes that are further compatible with the assumptions that either: (i) the reference population genotype matches the normal population genotype and the variant population has a copy number equal to the total copy number at the location and up to the major copy number of the variant allele; or (ii) the reference population has a copy number equal to the total copy number at the location and the variant population has 1 variant allele and a copy number equal to the total copy number at the location (“major copy number prior”). This approach advantageously strikes a good balance between accounting for uncertainty in the genotypes of the populations while not considering too many states.

Instead or in addition to this, a set of one or more candidate joint genotypes may comprise any of the candidate joint genotypes that are compatible with the assumption that: each mutation is diploid and heterozygous (i.e. G_{V}=AB, G_{R}=AA) (“AB prior”). Instead or in addition to this, a set of one or more candidate joint genotypes may comprise any of the candidate joint genotypes that are compatible with the assumption that: each mutation is diploid and homozygous (i.e. G_{V}=BB, G_{R}=AA) (“BB prior”). Instead or in addition to this, a set of one or more candidate joint genotypes may comprise any of the candidate joint genotypes that are compatible with the assumption that: the genotype of the variant population has the predicted total copy number at the region of the mutation, with exactly one mutant allele (i.e. G_{V}=(A)*mB where m=total copy number−1) (“no zygosity prior”). Instead or in addition to this, a set of one or more candidate joint genotypes may comprise any of the candidate joint genotypes that are compatible with the assumption that: the genotype of the variant population has the predicted total copy number at the region of the mutation, with at least one mutant allele, and the reference population is either AA or the genotype with a copy number equal to the predicted total copy number and no variant allele (i.e. G_{R}=(A)*n where n is the total copy number, G_{V}=(A)*m(B)*I where m+I=n and I>1) (“total copy number prior”). Instead or in addition to this, a set of one or more candidate joint genotypes may comprise any of the candidate joint genotypes that are compatible with the assumption that: the genotype of the variant population has a number of mutant alleles corresponding to either the major copy number or the minor copy number (“parental mode”).

The probability of observing the sequence data may combine a plurality of probabilities of observing the sequence data in view of a respective tumour fraction and a corresponding set of one or more candidate joint genotypes for at least one of the one or more samples, optionally wherein the method comprises obtaining, for at least one or the one or more samples, a plurality of estimates of the tumour fraction, and a plurality of corresponding sets of one or more candidate joint genotypes. Thus, the method may comprise obtaining, for at least one sample, a plurality of estimates of the tumour fraction. This may comprise comprises determining, by a processor, a plurality of estimates of the tumour fraction and a corresponding plurality of allele specific copy numbers that are compatible with the sequence data, and determining, by the processor, a plurality of sets of one or more candidate joint genotypes associated with said plurality of allele specific copy numbers.

The present method is advantageously able to determine a probability of a mutation being clonal which takes into account a plurality of possible tumour fractions and corresponding sets of candidate joint genotypes. In other words, the present method is able to obtain a probability of a mutation being clonal which integrates over a plurality of copy number solutions from which tumour fractions and candidate joint genotypes can be obtained. By contrast, prior art approaches typically rely on a single estimate of tumour purity and allele-specific copy numbers (from which candidate joint genotypes can be obtained), which is often manually selected according to expert defined optimality criteria. The step of selecting a copy number solution that is deemed optimal is highly error prone, and the output of methods that rely on single solutions is likely to change significantly depending on the solution.

Thus, advantageously, the probability of observing the sequence data (likelihood of the sequence data) may be calculated over a plurality of sets of candidate genotypes and corresponding tumour fraction estimates (e.g. as a sum of probabilities comprising a term for each copy number solution, see equations (3b), (4b)), the contribution of which may be weighted for example to reflect the confidence in the copy number solution from which the tumour fraction estimate and set of candidate genotypes were obtained. The weights of the contributions of the copy number solutions considered suitably sum to 1, such that the total probability reflects the relative contributions of the different copy number solutions considered. When a single copy number solution is used, it may be assigned a weight of 1 (i.e. no sum may be obtained).

The method may further comprise repeating the method for a plurality of tumour-specific mutations identified in the subject. The method may further comprise ranking or otherwise prioritising the plurality of tumour-specific mutations at least in part based on their determined likelihood of being clonal in the subject.

Further, other methods for determining the probability of a mutation being clonal may be used at step 222, which make use of a prior probability of the mutation being clonal determined using a method as described herein. For example, any of the methods described in WO 2016/16174085, Landau et al. (2013), or Roth et al. (2014) may be used, by replacing the respective uninformative priors used in these methods (typically U(0,1), i.e. uniform distributions) with informative priors as described herein (e.g. prior probabilities that take into account the mutation signatures that likely generated the mutation and the probability that a mutation generated by said mutational signatures is clonal).

### Applications

The above methods find applications in the context of cancer diagnostic, prognostic and therapeutic approaches. In particular, the above methods may be used to provide immunotherapies that target clonal neoantigens. Thus, also described herein are methods of providing an immunotherapy for a subject, the method comprising identifying one or more clonal neoantigens from one or more samples from the subject.

FIG. 3 illustrates schematically an exemplary method of providing an immunotherapy. At optional step 310, one or more samples comprising tumour genetic material and one or more germline samples are obtained from a subject. The subject may be a subject that has been diagnosed as having cancer, and may be (but does not need to be) the same subject for which the immunotherapy is provided. At step 312, a list of candidate clonal neoantigens is obtained using the methods described herein, for example by reference to FIG. 2. The list may comprise a single neoantigen, or a plurality of neoantigens. Preferably, the list comprises a plurality of neoantigens. At step 314, an immunotherapy that targets at least one (and optionally a plurality) of the candidate neoantigens is designed. Designing such an immunotherapy may comprise identifying one or more candidate peptides for each of the candidate clonal neoantigens (step 314A). For example, a plurality of peptides may be designed for at least one of the candidate clonal neoantigens, which differ in their lengths and/or the location of a sequence variation that characterises the neoantigen compared to the corresponding germline peptide. At step 314B, the one or more peptides identified may be tested in vitro and or in silico to evaluate one or more properties such as their immunogenicity, likelihood of being displayed by a MHC molecule, etc. At optional step 314C, one or more of the peptides may be selected, for example based on the results of step 314B.

At step 316, the selected peptides may be obtained. Peptides with selected sequences may be obtained using any method known in the art such as e.g. using an expression system or by direct synthesis. At step 318, an immunotherapy may be produced using the one or more candidate peptides. The immunotherapy may comprise the one or more candidate peptides or material sufficient for their expression (e.g. in the case of an immunogenic composition or vaccine), or may comprise molecules or cells that have been obtained using the candidate peptides (e.g. in the case of therapeutic antibodies that selectively bind the candidate peptides, or immune cells that specifically recognise the candidate peptides). At optional step 320, the immunotherapy may be administered to a subject, which is preferably the subject from which the samples used to identify the clonal neoantigens have been obtained. An example of producing an immunotherapy comprising a T cell population selectively enriched with T cells that recognise one or more clonal neoantigens will be described. At step 318A, a population of T cells may be obtained. The T cells may be obtained from the subject to be treated, but do not need to be. The T cells may be obtained from a tumour sample, from a blood sample, or from any other tissue sample. At step 318B, a population of dendritic cells may be obtained. For example, a population of dendritic cells may be derived from mononuclear cells (e.g. peripheral blood mononuclear cells, PBMCs) from the subject to be treated. At step 318C, the population of dendritic cells may be pulsed with the candidate peptides. At step 318D, the T cell population may be selectively expanded using the population of pulsed dendritic cells. Additional expansion factors such as e.g. cytokines or stimulating antibodies may be used.

Thus, the disclosure also provides a T cell composition comprising a T cell population selectively enriched with T cells that recognise one or more clonal neoantigens, wherein the one or more clonal neoantigens have been identified using any of the methods described herein.

In a T cell composition as described herein the expanded population of neoantigen-reactive T cells may have a higher activity than the population of T cells which have not been expanded, as measured by the response of the T cell population to restimulation with a neoantigen peptide. Activity may be measured by cytokine production, and wherein a higher activity is a 5-10 fold or greater increase in activity.

References to a plurality of clonal neoantigens may refer to a plurality of peptides or proteins each comprising a different tumour-specific mutation that gives rise to a neoantigen. Said plurality may be from 2 to 250, from 3 to 200, from 4 to 150, or from 5 to 100 tumour-specific mutations, for example from 5 to 75 or from 10 to 50 tumour-specific mutations. Each tumour-specific mutation may be represented by one or more clonal neoantigen peptides. In other words, a plurality of clonal neoantigens may comprise a plurality of different peptides, some of which comprise a sequence that includes the same tumour-specific mutation (for example at different positions within the sequence of the peptide, or within peptides of varying lengths).

A T cell population that is produced in accordance with the present disclosure will have an increased number or proportion of T cells that target one or more neoantigens that are predicted to be clonal. That is to say, the composition of the T cell population will differ from that of a “native” T cell population (i.e. a population that has not undergone the expansion steps discussed herein), in that the percentage or proportion of T cells that target a neoantigen that is predicted to be clonal will be increased. The T cell population according to the disclosure may have at least about 0.2, 0.3, 0.4, 0.5, 0 6, 0 7, 0 8, 0 9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100% T cells that target a neoantigen that is predicted to be clonal.

The immunotherapies described herein may be used in the treatment of cancer. Thus, the disclosure also provides a method of treating cancer in a subject comprising administering an immunotherapeutic composition as described herein to the subject.

Additionally, the presence of clonal neoantigens has been shown to be associated with improved prognosis in cancer. Thus, also described herein are methods of providing a prognosis for a subject that has been diagnosed as having a cancer, the method comprising determining the fraction and/or number of clonal neoantigens in one or more tumour samples from the subject.

Suitably, in any embodiment of any aspect described herein, the cancer may be ovarian cancer, breast cancer, endometrial cancer, kidney cancer (renal cell), lung cancer (small cell, non-small cell and mesothelioma), brain cancer (gliomas, astrocytomas, glioblastomas), melanoma, merkel cell carcinoma, clear cell renal cell carcinoma (ccRCC), lymphoma, small bowel cancers (duodenal and jejunal), leukemia, pancreatic cancer, hepatobiliary tumours, germ cell cancers, prostate cancer, head and neck cancers, thyroid cancer and sarcomas. For example, the cancer may be lung cancer, such as lung adenocarcinoma or lung squamous-cell carcinoma. As another example, the cancer may be melanoma. In embodiments, the cancer may be selected from melanoma, merkel cell carcinoma, renal cancer, non-small cell lung cancer (NSCLC), urothelial carcinoma of the bladder (BLAC) and head and neck squamous cell carcinoma (HNSC) and microsatellite instability (MSI)-high cancers. In some embodiments, the cancer is non-small cell lung cancer (NSCLC). In other embodiments, the cancer is melanoma.

Treatment using the compositions and methods of the present disclosure may also encompass targeting circulating tumour cells and/or metastases derived from the tumour. Treatment according to the present disclosure targeting one or more neoantigens may help prevent the evolution of therapy resistant tumour cells which may occur with standard approaches such as chemotherapy, radiotherapy, or non-specific immunotherapy. The methods and uses for treating cancer described herein may be performed in combination with additional cancer therapies. In particular, the T cell compositions described herein may be administered in combination with immune checkpoint intervention, co-stimulatory antibodies, chemotherapy and/or radiotherapy, targeted therapy or monoclonal antibody therapy. ‘In combination’ may refer to administration of the additional therapy before, at the same time as or after administration of the T cell composition as described herein.

The disclosure also provides a method for producing an immunotherapeutic composition, the method comprising identifying a neoantigen as likely to be clonal and producing an immunotherapeutic composition that targets the neoantigen.

Also described herein is a method of treating a subject that has been diagnosed as having cancer, the method comprising: identifying one or more clonal neoantigens by: identifying a plurality of tumour-specific mutations in the subject; determining whether one or more of the tumour-specific mutations is likely to be clonal in the subject; selecting one or more of the tumour-specific mutations as candidate clonal neoantigens, wherein a candidate clonal neoantigen is a tumour-specific mutation that satisfies at least one or more predetermined criteria on whether the tumour-specific mutation is likely to be clonal; and treating the subject with an immunotherapy that targets one or more of the selected candidate clonal neoantigens; wherein determining whether a tumour-specific mutation is likely to be clonal in a subject is performed using the methods described herein. For example, determining whether a tumour-specific mutation is likely to be clonal in a subject may comprise: obtaining, by a processor, sequence data from one or more samples from the subject comprising tumour genetic material, the sequence data comprising for each of the one or more samples, at least two of: the number of reads in the sample that show the tumour-specific mutation (d_{b}), the number of reads in the sample that show the corresponding germline allele, and the total number of reads at the location of the tumour-specific mutation (d), determining, by the processor, a prior probability of the mutation being clonal using a method as described herein, and determining, by the processor, the likelihood that the tumour-specific mutation is clonal as a posterior probability depending on the prior probability of the mutation being clonal. The posterior probability may further depend on the probabilities of observing the sequence data if the tumour-specific mutation is (i) clonal and (ii) non-clonal, in view of a tumour fraction for each of the one or more samples and one or more candidate joint genotypes each comprising a genotype at the location of the tumour-specific mutation for a normal population, a reference tumour population that does not comprise the tumour-specific mutation and a variant tumour cell population that comprises the tumour-specific mutation.

The candidate clonal neoantigens may be selected as tumour-specific mutations that further satisfy at least one or more predetermined criteria on whether the tumour-specific mutation is likely to give rise to a neoantigen. The step of selecting, by said processor, one or more of the tumour-specific mutations as candidate clonal neoantigens, may comprise determining whether the one or more tumour specific mutations satisfy one or more criteria on whether the tumour-specific mutation is likely to give rise to a neoantigen selected from: the mutation being associated with an expression product that is expressed in tumour cells, the mutation being predicted to result in a protein or peptide that is not expressed in the normal cells of the subject, the mutation being predicted to result in at least one peptide that is likely to be presented by an MHC molecule, the mutation being predicted to result in at least one peptide that is likely to be presented by an MHC allele that is known to be present in the subject, and the mutation being predicted to result in a protein or peptide that is immunogenic. The step of selecting, by said processor, one or more of the tumour-specific mutations as candidate clonal neoantigens, may comprise determining, by said processor, whether the one or more tumour specific mutations satisfy one or more predetermined criteria on whether the tumour-specific mutation is likely to be clonal selected from: the mutation having a likelihood of being clonal above a predetermined threshold, the mutation having a likelihood of being clonal that is above a threshold set adaptively to select a predetermined number of tumour-specific mutations with the highest likelihoods of being clonal amongst the tumour-specific mutations for which a likelihood was determined, and having a likelihood of being clonal that is above a threshold set adaptively to select a predetermined top percentile of tumour-specific mutations amongst the tumour-specific mutations for which a likelihood was determined.

The immunotherapy that targets the one or more of the selected clonal neoantigens may be an immunogenic composition, a composition comprising immune cells or a therapeutic antibody. The immunotherapy may be a composition comprising T cells that recognise at least one of the one or more of the selected clonal neoantigens identified. The composition may be enriched for T cells that target at least one of the one or more of the selected clonal neoantigens identified. The method may comprise obtaining a population of T cells and expanding the population of T cells to increase the number or relative proportion of T cells that target at least one of the one or more of the selected clonal neoantigens identified.

### Systems

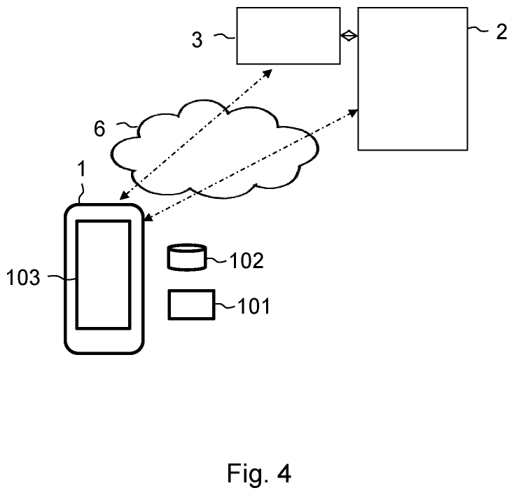

FIG. 4 shows an embodiment of a system for determining whether a tumour-specific mutation is likely to be clonal, determining a prior probability of a mutation being clonal, and/or identifying clonal neoantigens and/or for providing a prognosis or providing an immunotherapy based at least in part on the identified clonal neoantigens, according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 is communicably connected, such as e.g. through a network 6, to sequence data acquisition means 3, such as a sequencing machine, and/or to one or more databases 2 storing sequence data. The one or more databases may additionally store other types of information that may be used by the computing device 1, such as e.g. reference sequences, parameters, mutational signatures, etc. The computing device may be a smartphone, tablet, personal computer or other computing device. The computing device is configured to implement a method for determining whether a tumour specific mutation is likely to be clonal, as described herein. In alternative embodiments, the computing device 1 is configured to communicate with a remote computing device (not shown), which is itself configured to implement a method of determining whether a tumour specific mutation is likely to be clonal, as described herein. In such cases, the remote computing device may also be configured to send the result of the method to the computing device. Communication between the computing device 1 and the remote computing device may be through a wired or wireless connection, and may occur over a local or public network such as e.g. over the public internet or over WiFi.

The sequence data acquisition 3 means may be in wired connection with the computing device 1, or may be able to communicate through a wireless connection, such as e.g. through a network 6, as illustrated. The connection between the computing device 1 and the sequence data acquisition means 3 may be direct or indirect (such as e.g. through a remote computer). The sequence data acquisition means 3 are configured to acquire sequence data from nucleic acid samples, for example genomic DNA samples extracted from cells and/or tissue samples. In some embodiments, the sample may have been subject to one or more preprocessing steps such as DNA purification, fragmentation, library preparation, target sequence capture (such as e.g. exon capture and/or panel sequence capture). Preferably, the sample has not been subject to amplification, or when it has been subject to amplification this was done in the presence of amplification bias controlling means such as e.g. using unique molecular identifiers. Any sample preparation process that is suitable for use in the determination of a genomic copy number profile (whether whole genome or sequence specific) may be used within the context of the present disclosure. The sequence data acquisition means is preferably a next generation sequencer. The sequence data acquisition means 3 may be in direct or indirect connection with one or more databases 2, on which sequence data (raw or partially processed) may be stored.

The following is presented by way of example and is not to be construed as a limitation to the scope of the claims.

### EXAMPLES

These examples demonstrate the connection between clonality and mutational signatures, propose a framework for providing a prior probability of a mutation being clonal according to the present disclosure, demonstrate its use in combination with a particular method of identifying clonal mutations, and demonstrate that the use of the informative priors described herein improves the identification of clonal mutations.

### Example 1: Clonality Likelihood Varies by Mutational Signatures

In this example, the inventors investigate whether a meaningful association between consensus mutational signatures and clonality can be identified. They first establish an approach to associate mutational signatures to mutations for this purpose, including defining metrics that can be used as representative of the association between chosen mutational signatures and any mutations identified in a subject. They then investigate whether such metrics do correlate with clonality, and hence whether these could be used as an indication of the likelihood of a mutation being clonal prior to assessment of the evidence of clonality from sequencing data from the subject (prior probability of clonality).

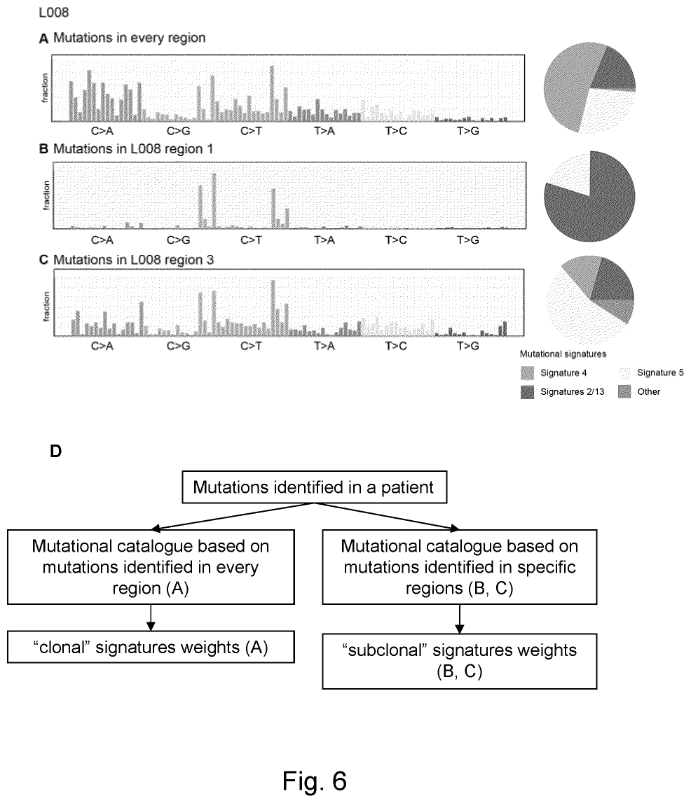

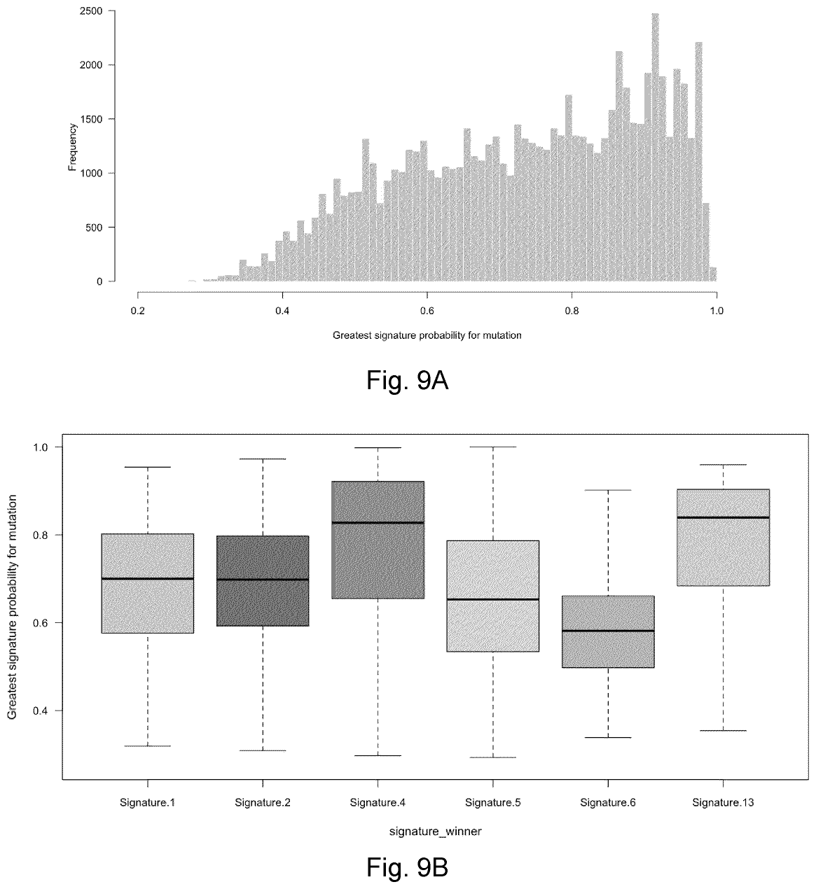

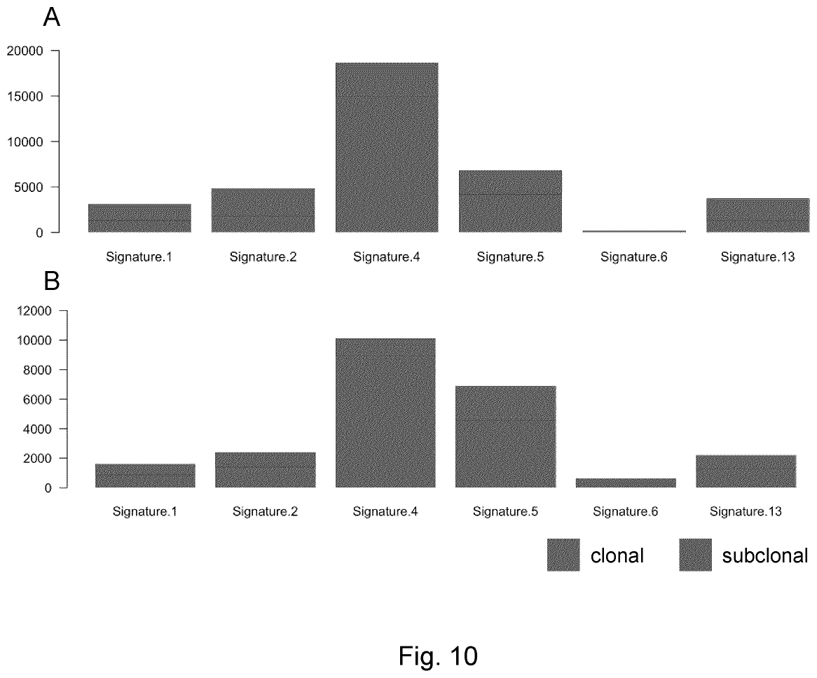

Methods Data for proof of principle. Data from selected lung cancer patients in the TRACERx cohort (see de Bruin et al., 2014) for which multi-region samples were available was analysed with deconstructSigs (Rosenthal et al., 2016) and mmsig (Rustad et al., 2021) with default values. For the results on FIG. 9, every sample in the TRACERx cohort (see de Bruin et al., 2014) was run through mmsig to determine the activity of each signature, then custom code was used to assign every mutation in a particular sample to the signature that most likely generated it (based on trinucleotide context and signature activities—see below). The same process was used for a subset of samples (training set) for the results on FIG. 10. The results on FIG. 6 relate to a single patient in the TRACERx cohort, analysed with deconstructSigs.

In silico data for benchmarking of signature tools. In silico data was obtained by creating a mock tumor with mutations at trinucleotide contexts specified using a predetermined set of signatures of interest and their weights (+ variance), and a predetermined number of mutations. The mock Lung+APOBEC tumors used aging, smoking, and APOBEC signatures “active” in its generation. Similarly, the SKCM+therapy tumors would have aging, UV radiation, and an alkylating agent active. The estimated signature weight obtained for this mock tumour using each of a plurality of tools was then compared to the true signature weight that was introduced in silico.

Signature tools used. The following tools were assessed: sigLASSO (Li et al., 2020), deconstructSigs (Rosenthal et al., 2016), sigLASSO with a zero prior instead of the default prior for the weights of the respective mutational signatures containing a vector of 1s (in order to compensate for not including all signatures in a catalogue, which is what sigLASSO was designed for), mmsig (Rustad et al., 2021), sigprofiler (Bergstrom et al., 2019). All of these tools were run with default parameters as set out in the respective publications above.

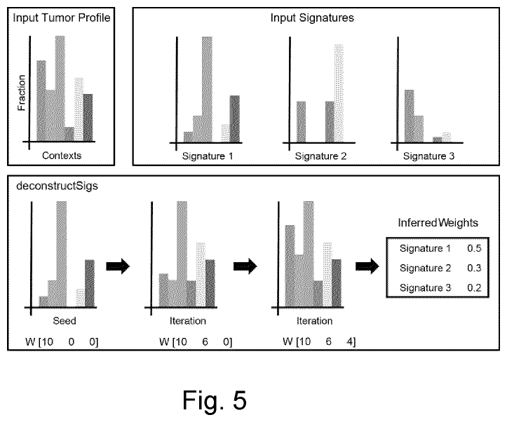

Associating mutation signatures to single mutations. Mmsig and deconstructSigs were used to quantify the relative weights of selected COSMIC signatures (Alexandrov et al., 2020) in TRACERx samples. The following COSMIC signatures were used: smoking (signature 4), APOBEC (signature 13 and signature 2), alkylating agent (signature 11), clock-like/ageing (signature 5 and signature 1). When analysing skin cutaneous melanoma (SKCM) samples an additional signature associated with UV exposure (signature 7), was also used. MMsig and deconstructSigs provide as output the relative weights of each of these signatures in a sample. Both of these tools were run with bootstrapping to assign a confidence interval to each of these weights (using the default approach of running the analysis 1000 times to generate the confidence intervals for the output weights of each signature). Based on the weights obtained for the signatures, custom code was used to assign a mutational signature to each mutation in each tumour sample as the mutational signature that had the highest probability of generating that mutation based on its trinucleotide context. In other words, the estimated probability that each signature contributed to each mutation class for each sample was obtained and these were used to assign a mutational signature to each mutation. In particular, the relative weights (wⁿₛ) assigned to each signature n in a sample s provides an indication of the number or proportion of mutations that are assigned to the signature n (the weights representing the proportion of the total number of mutations in sample s that are assignable to the respective signatures). The signature themselves provide information about the proportion of mutations in each mutation class k that is expected to result from the activity of the mutational signature n (p^{k}ₙ—see FIG. 5, Input signatures comprising fractions of mutations for each of a plurality of contexts, i.e. proportions of mutations for each of a plurality of mutation classes). Thus, for a mutation in a particular class, a probability of this mutation class k in a sample s to be generated by signature n can be calculated as (wⁿₛ p^{k}ₙ). When the weights assigned to each signature are exposures (e.g. when using NMF) representing the number of mutations assigned to the signature, this can be normalised by the total number of mutations in the sample so that the probabilities still sum to 1. Custom code was also used to compute a confidence interval for these by bootstrapping. Each mutation in each tumour sample was assigned a mutational signature that had the highest probability of generating that mutation based on its trinucleotide context..

Signature weights distribution in different lung cancer stages. The respective weights for each signature in each sample calculated as explained above were compared across different cohorts of lung cancer patients separated by stage (from the TRACERx cohort—stage information as recorded in Jamal-Hanjani et al (2017)).

Signature analysis for clonal vs subclonal mutations. TRACERx LUAD and LUSC samples from patients with multi-region samples were analysed using mmsig as described above and a single mutational signature was assigned to each mutation in each sample as the most likely mutational signature (signature with highest likelihood). The total number of mutations associated with each signature was then separated between two groups: mutations that were identified as likely present in every cancer cell (clonal mutations) by Jamal-Hanjani et al. (2017) and those that were identified as present in only a subset of cancer cells (subclonal mutations).

### Results

In a first proof of principle study, data from TRACERx was analysed using deconstructsigs to quantify the contribution of known signatures to the mutational profiles observed in these samples. As illustrated on FIG. 5 (which is adapted from Rosenthal et al., 2016), deconstructsigs takes as input a mutational profile for a sample and a plurality of mutational signatures (such as e.g. consensus signatures as provided in e.g. the COSMIC database https://cancer.sanger.ac.uk/signatures/or the signal database, https://signal.mutationalsignatures.com/) produces as output a set of weights that quantify the relative contribution of each of the input signatures to the mutational profile in the sample. In this proof of principle, the following reference signatures were used: COSMIC signature 4 (associated with smoking), COSMIC signatures 13 and 2 (associated with APOBEC activity), signature 5 (clock-like signature). These signatures were chosen because they are postulated to be possibly active in lung cancer. Deconstructsigs further uses an “other” category which is a “catch all” category to represent all mutational signatures that are not included in the particular analysis. As illustrated on FIG. 6D, for each patient in the cohort two sets of artificial mutational catalogues were created: one based on mutations that were consistently identified in all regions of the patient's tumour (FIG. 6A which illustrates the result for an example patient in the TRACERx cohort), and one based on the mutations that were identified in each of the separate regions of the patient's tumour (FIGS. 6B-C). These were then analysed separately with deconstructsigs. An example result of this analysis is shown on FIG. 6A-C, from which it can be seen that the relative contributions of the various mutational signatures varies between tumour regions and between likely clonal and subclonal mutations. This indicates that different mutational processes may have been active when the truncal population of cancer cells was emerging and when the various branch clones emerged.

Various approaches to assign weights quantifying relative contributions of signatures to a mutational catalogue were then benchmarked using in silico data. Lung cancer data simulated to include APOBEC signature induced mutations (signatures 2, 13), and SKCM data simulated to include temozolomide induced mutations (signature 11) were analysed using sigLASSO (Li et al., 2020), deconstructSigs (Rosenthal et al., 2016), mmsig (Rustad et al., 2021), sigprofiler (Bergstrom et al., 2019). For the in silico lung cancer samples, the following signatures were included: signatures 1 (clock-like signature, spontaneous deamination of 5′-methylcytosine), 2, 13 (activity of APOBEC family of cytidine deaminase), 4 (tobacco smoking), 5 (clock-like signature). For the in silico melanoma cancer samples, the following signatures were included: signatures 1 (clock-like signature, spontaneous deamination of 5′-methylcytosine), 7 (ultraviolet light exposure), 11 (temozolomide treatment), 5 (clock-like signature). The relative weights of each of these signatures were then compared and the tool that best approximated the known signature weights used in the in silico design of the samples was considered to perform best.

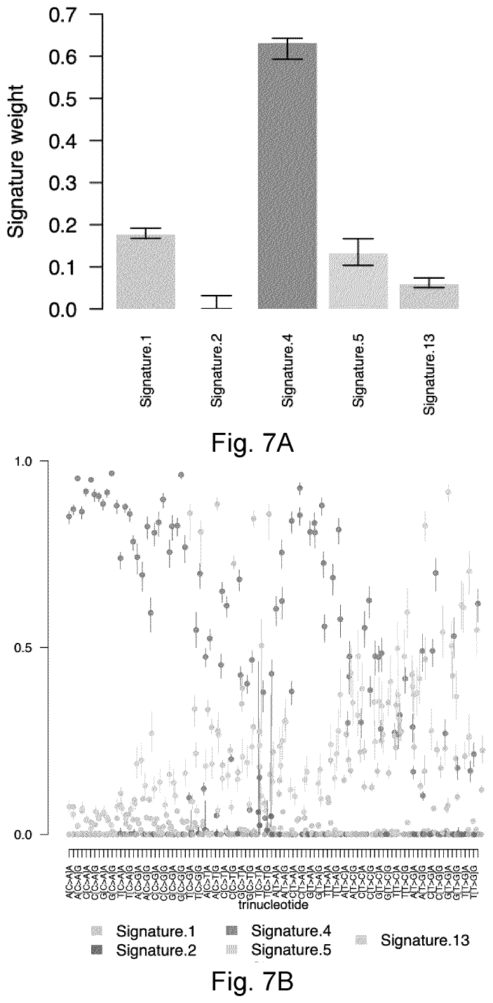

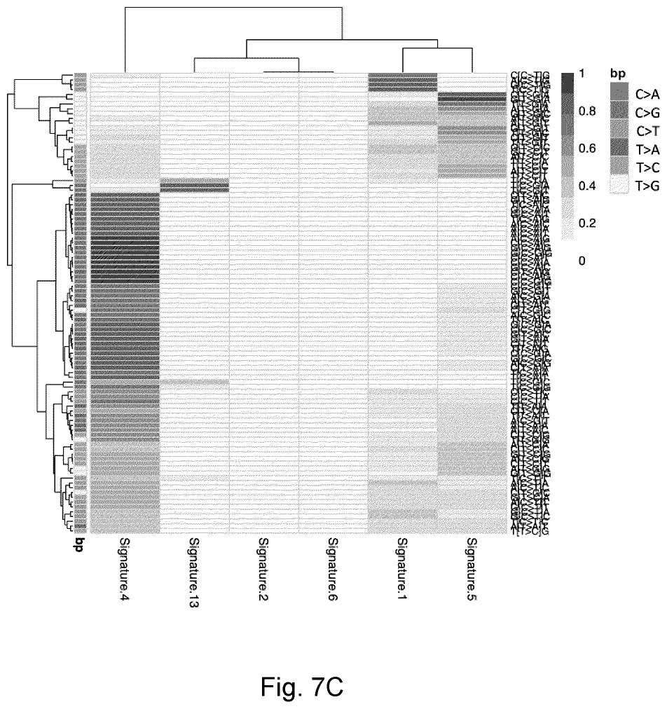

Bootstrapping was used to assign a confidence interval for signature weight assignment for samples. A new approach was developed to assign signature weights and confidence intervals for each category of mutation. The approach is applicable to any method that assigns signature weights or exposures for samples (including any of mmsig, sigLASSO, deconstructSigs, sigprofiler). To generate the present data, mmsig was used. This was evaluated on TRACERx samples using signatures 1, 2, 4, 5 and 13. The signatures were chosen based on associations between signatures and cancer types in COSMIC. FIG. 7 shows example results for a single sample (all TRACERx samples were analysed in the same manner). Panel A shows the signature weight and bootstrap confidence intervals for each signature for the sample. This plot shows that the mutational catalogue for this sample is dominated by signature 4 (smoking). Panel B shows the mutational category weight estimate and corresponding bootstrap confidence intervals obtained for this sample. Panel C shows the mutational category weight estimates in a heatmap. The data shows that for most mutation categories a single signature is dominant. For this sample this is the smoking signature in many cases but not in all cases. In particular, any C>A mutation in these samples is likely to be associated with signature 4 (smoking). However, T[C>G]A and T[C>G]G mutations are very likely to be associated with signature 13 (APOBEC activity), and most T>G or T>C mutations are likely to be associated with signature 5 (clock-like signature).

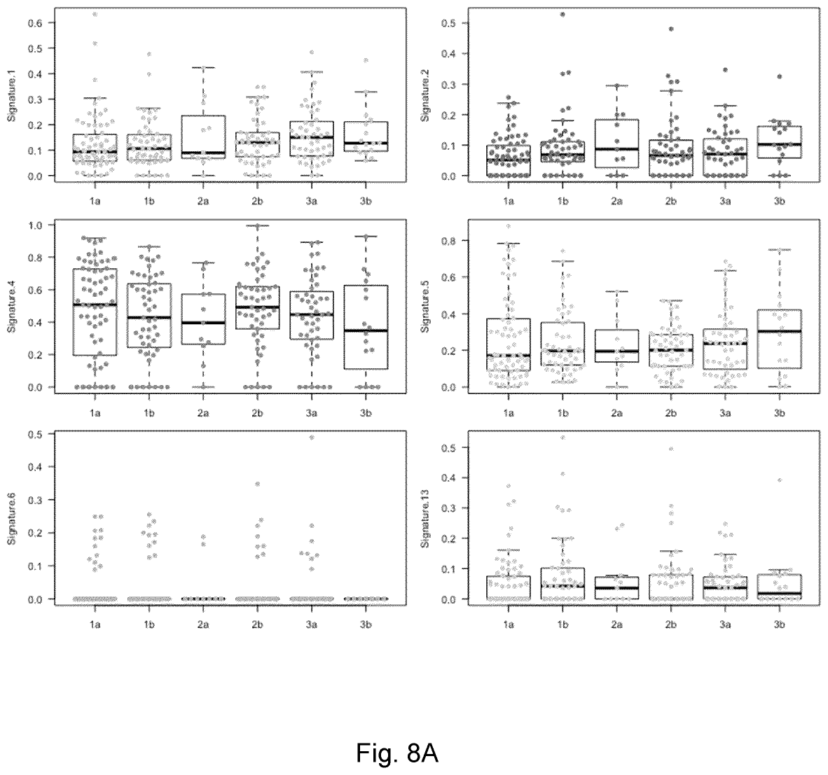

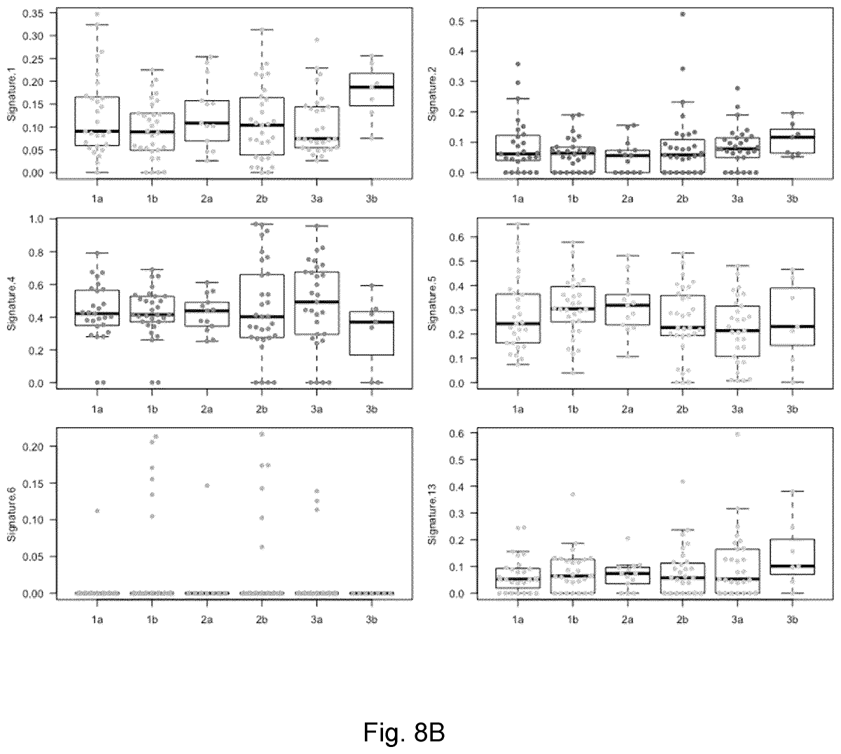

In order to test the robustness of the signal identified, the signature weights per sample were calculated using mmsig for all LUAD and LUSC samples in the TRACERx cohort. These estimates were then separated by cancer stage in order to see whether the cancer stage constituted a confounding factor in the relationship between signature activity and timing of occurrence of a mutation (truncal/clonal vs branch/subclonal). The results of this analysis are shown on FIGS. 8A (LUAD) and 8B (LUSC). They show no significant association between cancer stage and signature weight, i.e. none of the distributions of weights shown were significantly different from each other, indicating that samples from different cancer stages can still be analysed together for the purpose of determining associations between mutational signatures and clonality.

The association between clonality of mutations and likely signature of origin was then evaluated over the entire TRACERx cohort. In particular, each single mutation was assigned a single signature as the one with highest likelihood for the mutation class in which the mutation belongs (“top signature”). A histogram of the weights for these signatures across all mutations is shown on FIG. 9A. This shows that for the vast majority of mutations the top signature has a weight of over 0.5. Boxplots showing the distributions of weights for these signatures across all mutations, separated by top signature, is shown on FIG. 9B. This shows that in this cohort, signature 4 (smoking) is associated with the most confident assignments (greatest probability), although the other signatures chosen, on particular signatures 1, 2 and 13, all have relatively high distributions. In other words, if a mutation was thought to have arisen due to signature 4, then it tended to have a high probability of doing so. Finally, for each set of mutations assigned to a top signature, the clonal vs subclonal status (as annotated in the TRACERx data, where clonal is presumed to be found in all cancer cells, and subclonal is found in a subset of cancer cells) and the number of mutations in each category was plotted on FIG. 10 (A=LUAD, B=LUSC). This shows that some signatures are more likely to be associated with clonal mutations (e.g. signatures 4 and 5) whereas other signatures are equally likely to be associated with clonal and nonclonal mutations. This is in line with the biological assumption that some mutational processes are likely to be active throughout the evolution of a cancer (e.g. clock-like signatures), whereas others are likely to be particularly active early on in cancer evolution (e.g. smoking signature) or later one in cancer evolution (e.g. treatment associated signatures). In other words, the likelihood of clonality varies by mutational signatures across a cohort of patients, and hence assignment of likely mutational signatures weights for individual mutations may be used as a prior indication of whether a mutation in a patient is likely to be clonal in the absence of any sequencing data from the patient to evaluate clonality for the mutation.

### Example 2: Development of a Framework for Providing a Prior Probability of Clonality

In this example, a new framework to derive a prior probability of a particular mutation being clonal using the insights generated in Example 1 is presented. This approach is demonstrated in the context of a particular method for determining the probability that a mutation is clonal (described below, labelled as “ACE”, and in WO 2022/207925).

### Methods

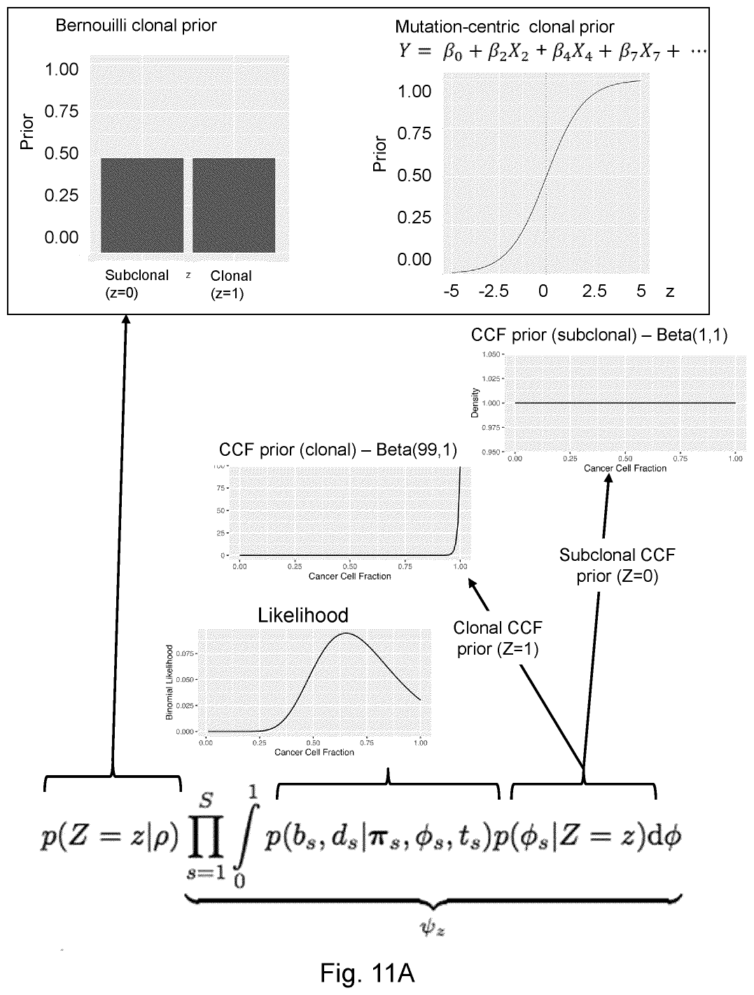

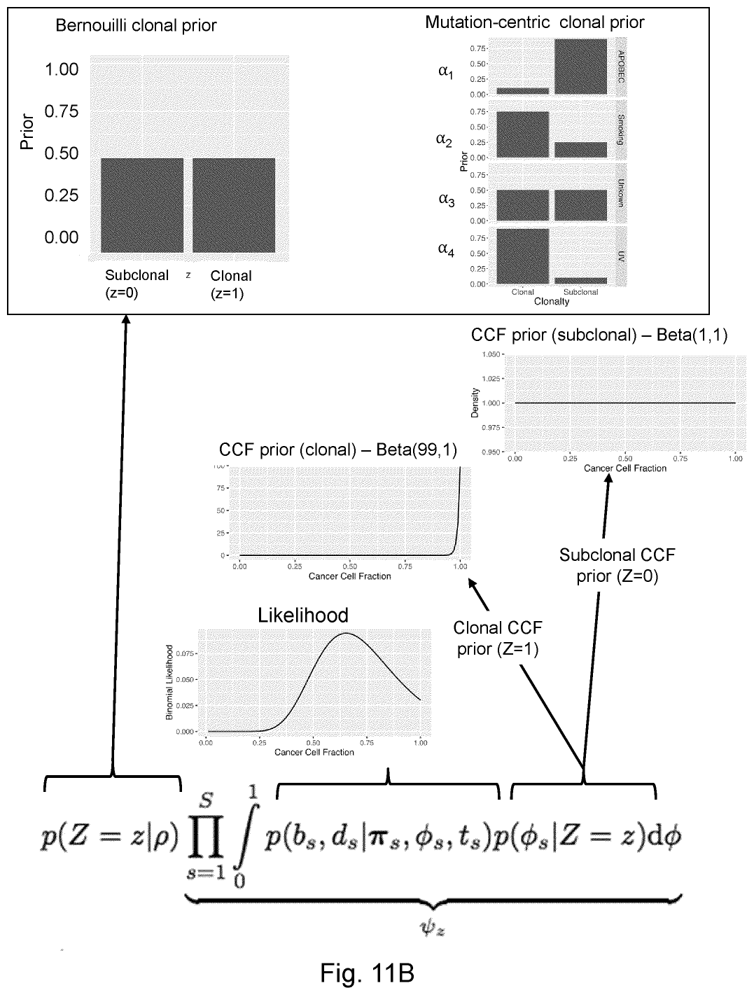

Prior estimation using logistic regression. A logistic regression model was trained to predict a clonal prior (log odds probability of a mutation being clonal, Y on FIG. 11A) as a function of the log odds of mutations assigned to each mutational signature n being clonal (βₙ, where n refers to the n^{th} mutational signature, e.g. signature 4 (smoking), signature 2 (APOBEC), signature 7 (UV), etc.), and the weight of each signature for the particular mutation (Xₙ on FIG. 11A). Thus, the training process determined the coefficients βₙ (and β₀) based on training data as described below. The weights Xₙ refer to the above determined point estimates of the probability that a mutation is associated with a particular signature (see Example 1). Note that ΣXₙ=1 in this example. Thus, the prior estimation takes into account the uncertainty of signature assignment through the use of the weights for the multiple signatures considered (as a mutation that can be confidently assigned to a single signature will have a high weight for that particular signature and a low weight for other signatures). The distribution around the point estimates (estimated in the examples above through the use of bootstrapping), and the corresponding confidence interval estimated in Example 1 are not used in this particular example. The Y-intercept term (β₀) captures unaccounted bias in the model and ensures that the mean residual in the model sums to 1. Note that while the illustrated equation only shows terms for mutational signatures, the approach is extendable to any factor that can be associated with a mutation and that may be associated with a priori likelihood of clonality. This includes e.g. whether the mutation is occurred before or after genome-doubling (as genome double tends to be early mutations before genome-double have a high probability of being clonal; Jamal-Hanjani et al. 2020), the driver status of the gene in which the mutation resides (where driver mutations may be more likely to be clonal), ethnicity (see Li et al. bioRxiv. 2020), age (see Li et al. 2022) etc. Each of these factors may be taken into account by including a term in the model that reflects the log odds of mutations falling in the respective category being clonal (e.g lods of driver mutation in a particular gene being clonal/lods of non-driver mutation being clonal, lods of mutations being clonal for ethnicity considered, lods of genome duplicated mutation being clonal, etc). The point estimates of the probabilistic values per mutation (Xₙ determined as explained in Example 1) are used to training the logistic regression model (in additional to the clonal label of each mutation), using training data with known clonal labels for mutations. This training process enables to determine the value of the coefficients for each mutation signature in the model (pA (and po)). The larger the coefficient the higher the probability that a mutation assigned to this mutation signature is associated with clonality. These coefficients have confidence interval values associated with the training process. The model coefficient confidence values are not directly used when applying the trained model to new data. However, they are used to determine statistically whether a mutation signature significantly contributes to the determination of a clonal/subclonal mutation. In other words, the confidence interval around the model coefficients provide an indication of whether a signature is significantly associated with clonality in the training data. This may depend on the training data, and in particular on the cancer type, as different mutational signatures are expected to be active at different times in the evolution of different types of cancers. For example, in melanoma the UV signature may be a marker of early mutations, whereas on non-skin cancers the same signature may be a marker of metastasis. Further, this information can be sued to select mutational signature to include in a particular model. For example, logistic regression models can be trained using different sets of candidate signatures and those that are associated with model coefficients that are most significantly associated with clonality may be used in the final model that is deployed.

Prior estimation using weighted clonal prior. As an alternative to the model described above, a simpler model using a weighted estimate was tested. This calculates the clonal prior for a particular mutation as a weighted combination of the signature assignment weights (which sum to 1) and the probability of mutations assigned with each mutational signature n being clonal, in the particular disease considered. This was calculated for a candidate mutation as the sum across signatures of the probability of the mutation belonging to the nth signature (αₙ—where Σαₙ=1) and the probability of a mutation assigned to the signature n being clonal (Pₙ), i.e. Σ₁ⁿα_{n·}Pₙ (see FIG. 11B). The probability of a mutation assigned to the signature n being clonal in the particular disease was obtained by assigning a mutational signature to each mutation with known clonal status in a training cohort (signature with highest weight) and determining the proportion of mutations assigned to the signature that were clonal. This approach cannot be extended to include additional factors as the weights must sum to 1 (which is the case for mutational signatures assignment weights).

In both cases a separate model was obtained for each disease type (in this example, NSCLC and melanoma). Data from TRACERx100 (NSCLC—77 patients for training, 23 patients for testing, each patient being represented by multiple samples) and TCGA (melanoma, 186 samples for training, 55 samples for testing) was used to train the logistic regression model/obtain the weights for the weighted clonal prior. The following COSMIC signatures were used: 1, 2, 4, 5, 6, 7, 11, 13 and 17 (although the final set of signatures included in the model depended on the signatures that were actually assigned to mutations in the training data—no mutations were assigned to UV signatures in the NSCLC data and no mutations were assigned to the smoking signature in the melanoma data). Signature weights for single mutations in these samples obtained using mmsig and the approach described in Example 1 were used. For the purpose of estimating the likelihood/log odds of mutations assigned to each signature being clonal (βₙ), clonality of individual mutations was determined using the clonality annotation in TRACERx for NSCLC samples, and on the TCGA data for melanoma with a criterion that the 95% cancer cell fraction confidence interval was ≥1 (as defined by ABSOLUTE).A binary value (clonal/non-clonal) was assigned to each mutation. The logistic regression model was trained using standard maximum likelihoods that selects coefficients that maximizes the likelihood of the observed data.

The remainder of the methods section below describes the Bayesian method for determining the probability of a mutation being clonal that is used in this example (ACE).

### Mutational Genotype Model

The data for the model is allele counts from N mutations (n=1, . . . N) from S samples (s=1, . . . , S). For simplicity, and because the method can analyse a single sample and mutation, the indices n for the mutation and s for the sample will not be explicitly included in the notations used this section. The model assumes that each mutation divides the set of cells that were sequenced into three sub-populations: (i) the normal cell population consisting of cells with healthy germline genomes (likely diploid in the region of the mutation); (ii) the reference cell population which consists of cancer cells without the mutation in question (may be aneuploid in the region of the mutation in question); and (iii) the variant cell population which consists of cancer cells with the mutation in question (may be aneuploid in the region of the mutation in question, may not have the same copy number in said region as the reference population). The term “mutation” is intended here in its broadest sense to refer to any genetic alteration that is detectable in sequence data, and particularly genomic sequence data. This includes in particular single nucleotide variants (SNVs), multiple nucleotide variants (MNVs), indels, etc.

Let G=(A, B, AA, AB, AAA, AABB, . . . ) be the set of all genotypes where A and B represent reference and variant alleles respectively. For example, AB would represent a heterozygous variant (comprising one reference/normal allele A and one variant allele B) with total copy number 2. Under this notation, in FIG. 4, the normal population has the genotype AA (where both A can be the same or different, i.e. the normal population may be homozygous or heterozygous, but both alleles are normal), the reference population has the genotype AAA (where the A alleles are selected from the A alleles of the normal population), and the variant population has the genotype AABB (where the A alleles are selected from the A alleles of the normal population and the B alleles are any non-reference alleles). We assume that the genotype of all cells within each sub-population is constant (i.e. by reference to FIG. 4, all cells in the normal population have the genotype AA, all cells in the reference population have the genotype AAA, and all cells in the variant population have the genotype AABB). Let G=(G_{H}; G_{R}; G_{V})ϵG³ be a vector where the entries are the genotype of the normal (healthy), reference and variant populations respectively (each of these individual genotypes will be referred to generically as “G” below). Let t be the proportion of cancer cells in the sample. This is often referred to as the tumour content, tumour purity or cellularity of the sample. Let ϕ be the proportion of cancer cells harbouring the mutation in the sample, that is the relative proportion of cancer cells in the variant population. This is often referred to as the cancer cell fraction (CCF) or cellular prevalence of the mutation. Let E be the assumed sequencing error rate. The following functions are defined:
a(G): G→ [symbol] is a function which maps a genotype to the number of A alleles (e.g., where G is AA, a(G)=2)
b(G): G→ [symbol] is a function which maps a genotype to the number of B alleles (e.g., where G is AA, b(G)=0)
c(G): G→ [symbol] is a function which maps a genotype to the total copy number at the locus (i.e. c(G)=a(G)+b(G); e.g. where G is AA, c(G)=2)
μ(G): G→ [symbol] is a function which maps a genotype to the value μ(G)=min{max{(b(G)/c(G)), ε}, (1−ε)}, which can be interpreted as the probability of sampling a read with the mutation from a population with genotype G.

Let ξ(G, ϕ, t) be the probability of sampling a read with the variant allele. Assuming that we have an infinite initial population of cells which are sampled when sequencing, the probability of sampling a read with a variant allele is roughly proportional to the number of copies of the variant allele in the input pool of DNA. More formally, accounting for sequencing error, the probability of sampling a variant allele (given a set of genotypes G, a tumour content t and a cancer cell fraction ϕ) is given by the following equation (equation (1)):

$$ξ ( G , ϕ , t ) = \frac{1}{T} ( 1 - t ) c ( {G}_{H} ) μ ( {G}_{H} ) + \frac{1}{T} t ( 1 - ϕ ) c ( {G}_{R} ) μ ( {G}_{R} ) + \frac{1}{T} t ϕ c ( {G}_{V} ) μ ( {G}_{V} ) \qquad ( 1 )$$

$$where T = ( 1 - t ) c ( {G}_{H} ) + t ( 1 - ϕ ) c ( {G}_{R} ) + t ϕ c ( {G}_{V} ) . \qquad ( 2 )$$

The variable ξ(G, ϕ, t) captures the sum of the number of copies of the variant allele originating from each genotype multiplied by the probability of sampling a read with a mutation from the genotype, normalised by the sum of the total number of copies of both alleles originating from each genotype.

The variable d is the total number of reads covering the mutation in the sample, of which d_{b}contain the mutant allele. Thus, the probability of observing these number of reads d, d_{b} (P(d, d_{b}|G, ϕ, t)) can be expressed with a Binomial model with parameters d_{b} and ξ(G, ϕ, t) (equation (3)). This is because the sum of m Bernouilli random variables with parameter p follow a Binomial distribution with parameters m, p². A Beta-binomial model with mean ξ(G, ϕ, t) and precision (inverse of variance) γ (equation (4)) can be used instead, for example if the data has more variance than can be explained by a Binomial model:

$$P ( d , {d}_{b} \vert G , ϕ , t ) = Binomial ( {d}_{b} \vert d , ξ ( G , ϕ , t ) ) \qquad ( 3 )$$

$$P ( d , {d}_{b} \vert G , ϕ , t , y ) = Beta Binomial ( {d}_{b} \vert d , ξ ( G , ϕ , t ) , y ) . \qquad ( 4 )$$

The parameters γ is set to 200 in the examples below, though other values are possible. So far, we have assumed that the genotypes of the sub-populations were known. In general this may be true for the healthy population (e.g. from a matched germline sample), but this is not true for the reference and the variant populations. Instead, it is typical to observe allele specific copy number estimates for the region overlapping a mutation. Using this information, we can elicit a prior over a set of plausible genotypes. We explain how to do this in the next section. For now assume we have a vector π of prior probabilities where πᵢ is the prior probability of the i^{th} plausible joint genotype, Gᵢ, of the populations. We can write the probability of the observed data marginalizing over all plausible genotypes as follows (equations (3a), (4a)):

$$P ( d , {d}_{b} \vert π , ϕ , t ) = {Σ}_{i} {π}_{i} Binomial ( {d}_{b} \vert d , ξ ( {G}_{i} , ϕ , t ) ) \qquad ( 3 a )$$

$$P ( d , {d}_{b} \vert π , ϕ , t , y ) = {Σ}_{i} {π}_{i} Beta Binomial ( {d}_{b} \vert d , ξ ( {G}_{i} , ϕ , t ) , y ) . \qquad ( 4 a )$$

In the subsequent sections, the notation Pr(d, d_{b}|π, ϕ, t) will be used to refer equally to the expression of equation (3a) and equation (4a). Note that ϕ and t are associated with individual samples so the notation above is a shorthand for ϕₛ and tₛ, respectively.

### Eliciting Mutational Genotype Priors

The above model uses either a known joint genotype, or prior probabilities π, where πᵢ is the prior probability of the i^{th} plausible joint genotype, Gᵢ, of the populations (i.e. Gᵢ is one possible combination of genotypes for the healthy, variant and reference populations). Various methods can be used to set potential genotype priors.

For example, one possible method can be referred to as the “major copy number” method. Let cₘₐⱼₒᵣ and cₘᵢₙₒᵣ denote the major and minor allele copy number for the region overlapping the mutation in the tumour sample. The method “major copy number method” considers two cases:
(a) In the first case, the mutation occurs before the copy number event. In this case the reference population genotype matches the normal population. We consider all possible mutational genotypes for the variant population with up to cₘₐⱼₒᵣ chromosomes containing the variant.
(b) In the second case, the mutation occurs after the copy number event. In this case the reference population has cₘₐⱼₒᵣ+cₘᵢₙₒᵣ reference alleles. The variant population has 1 variant allele and cₘₐⱼₒᵣ+cₘᵢₙₒᵣ−1 reference allele.

We set the prior weights to be equal for all possible mutational genotypes. For example suppose we have that cₘₐⱼₒᵣ=2 and cₘᵢₙₒᵣ=1 and the normal copy number is 2. We have the following possible genotypes:
G₁=(AA, AA, AAB)
G₂=(AA, AA, ABB)
G₃=(AA, AAA, AAB)
   each with a prior probability of 1/3. Note that if allele specific copy number is not available then cₘₐⱼₒᵣ can be set to the total copy number and cₘᵢₙₒᵣ to zero. This approach assumes that a mutation occurs only once, such that if more than one copy of the mutant allele is present in the variant population, then this occurred because the mutation preceded a copy number change at the locus and was subsequently amplified. This approach strikes a good balance between accounting for uncertainty in the genotypes of the populations while not considering too many states.

Alternative approaches may be used for setting the mutational genotype priors. Another possible approach is to simply assume that each mutation is diploid and heterozygous (i.e. the variant in the variant population only occurs on one of the two chromosomes, G=(G_{H}=AA, G_{R}=AA, G_{V}=AB)). This may be referred to as “AB prior”. Yet another simplistic approach is to assume that each mutation is diploid and homozygous (i.e. the variant in the variant population occurs on both of the two chromosomes, G=(G_{H}=AA, G_{R}=AA, G_{V}=BB)). This may be referred to as “BB prior”. Yet another possible simple approach is to assume that the genotype of the variant population has the predicted total copy number at the region of the mutation, with exactly one mutant allele (i.e. assuming that the total copy number is 3, G=(G_{H}=AA, G_{R}=AA, G_{V}=AAB), i.e. this results in considering only G₁ in the “major copy number” method above). This may be referred to as “no zygosity prior”. These approaches may be too simplistic in many cases as they essentially consider a single possible genotype.

Another possible approach is to assume that the genotype of the variant population has the predicted total copy number at the region of the mutation, with at least one mutant allele, and that the reference population is either AA or the genotype with a copy number equal to the predicted total copy number and no variant allele (with equal probability). This may be referred to as the “total copy number prior” and intuitively means that the genotype of the variant population at the locus has the predicted total copy number and may have any number (>0) of copies of the mutant allele (i.e. assuming that the total copy number is 3, the possible genotypes are, with equal probabilities, G₁=(G_{H}=AA, G_{R}=AA, G_{V}=AAB), G₂=(G_{H}=AA, G_{R}=AA, G_{V}=ABB), G₃=(G_{H}=AA, G_{R}=AA, G_{V}=BBB), G₄=(G_{H}=AA, G_{R}=AAA, G_{V}=AAB), i.e. this essentially ignores the major and minor copy number values and considers all possible genotypes with n copies—leading to an additional genotype being considered compared to the “major copy number” method above). Yet another approach that can be used is to “trust” the predicted number of major and minor alleles from the copy number caller, such that only genotypes that have a number of mutant alleles corresponding to either the major copy number or the minor copy number are considered. This may be referred to as the “parental” mode. For example, if major copy number=3, minor copy number=1, then this approach would consider the following possible genotypes, with equal probabilities: G₁=(AA, AA, AAAB), G₂=(AA, AA, ABBB), G₃=(AA, AAAA, AAAB) (i.e. either 1 or 3 mutated alleles in the variant population). By contrast, the “major copy number” approach “trusts” the range of the possible major copies, but not the absolute value of it, by considering all values between 1 and the predicted major copy number. With the example above of major copy number=3, minor copy number=1, this would lead to one more genotype being considered compared to the “parental” mode, i.e.: G₁=(AA, AA, AAAB), G₂=(AA, AA, AABB), G₃=(AA, AA, ABBB), G₄=(AA, AAAA, AAAB). Thus, the “major copy number” approach strikes a good balance between accounting for additional uncertainty from the copy number calls (compared to the “parental” approach) without having consider too much uncertainty (compared to the “total copy number” approach).

### Clonality Estimation Model

This section outlines the hierarchical Bayesian model for identifying ubiquitous mutations. Let Z be a Bernoulli variable which is one when a mutation is ubiquitous (assumed to be clonal) and zero otherwise. Let p be the prior probability that the mutation is ubiquitous. This is set to 0.5 in the examples below. As above, ϕ is the proportion of cancer cells harbouring the mutation in the sample. Thus, the model can be expressed as:

$$Z \vert ρ \sim Bernoulli ( Z \vert p ) \qquad ( 5 )$$

$$ϕ \vert Z \sim Beta ( ϕ \vert α = 1 , β = 1 ) for Z = 0 ; Beta ( ϕ \vert α , β = 1 ) for Z = 1 \qquad ( 6 )$$

$${d}_{b} , d \vert π , ϕ , t \sim Pr ( d , {d}_{b} \vert π , ϕ , t ) \qquad ( 7 )$$

where α is a parameter>1 in the distribution of ϕ|Z=1. This is set to α=99 in the examples below. A Beta distribution with parameters α=99 and β=1 is skewed towards 1, capturing the assumption that clonal mutations should be enriched for higher cancer cell fraction ϕ. Other values of the parameter a are possible, though values that capture this assumption are preferred. As mentioned above, the probability in equation (7) is given by equations (3)/(3a) or (4)/(4a).

The joint distribution can be expressed with the following equation (equation (8)):

$$p ( {d}_{b} , d , ϕ , Z = z \vert π , t , ρ ) = p ( Z = z \vert ρ ) Pr ( {d}_{b} , d \vert π , ϕ , t ) p ( ϕ \vert Z = z ) \qquad ( 8 )$$

for one sample, or for a plurality of samples:

$$p ( {d}_{b} , d , ϕ , Z = z \vert π , t , ρ ) = p ( Z = z \vert ρ ) {\prod }_{s = 1}^{S} Pr ( {d}_{b} , d \vert π , ϕ , t ) p ( ϕ \vert Z = z ) \qquad ( 8 a )$$

The proportion of cancer cells harbouring the mutation (ϕ) is unknown. However, we can express:

$$p ( {d}_{b} , d , ϕ , Z = z \vert π , t , ρ ) = {\int }_{0}^{1} P ( {d}_{b} , d , ϕ , Z = z \vert π , t , ρ ) d ϕ = P ( Z = z \vert ρ ) {\int }_{0}^{1} Pr ( {d}_{b} , d \vert π , ϕ , t ) p ( ϕ \vert Z = z ) d ϕ \qquad ( 9 )$$

for one sample, or for multiple samples:

$$p ( {d}_{b} , d , ϕ , Z = z \vert π , t , ρ ) = P ( Z = z \vert ρ ) {\prod }_{s = 1}^{S} {\int }_{0}^{1} Pr ( {d}_{b} , d \vert π , ϕ , t ) p ( ϕ \vert Z = z ) d ϕ . \qquad ( 9 a )$$

The quantity Πₛ₌₁^{S}∫₀¹ Pr(d_{b}, d|i, p, t) p(ϕ|Z=z)dϕ may be referred to as L_{z} (i.e. ψ_{z} and ψ₁ respectively referring to the likelihood of the data if the mutation is non clonal and if the mutation is clonal). As P(Z=z|ρ)=(1−ρ) for z=0 (i.e. the prior probability of Z=0, i.e. the mutation being classified as non-clonal, given a prior probability p of the mutation being clonal is equal to the prior probability of the mutation not being clonal), and P(Z=z|ρ)=ρ for z=1 (i.e. the prior probability of Z=1, i.e. the mutation being classified as clonal, given a prior probability of the mutation being clonal of p is equal to the prior probability of the mutation being clonal), it follows that:

$$p ( {d}_{b} , d \vert π , t , ρ ) = {\sum }_{z = 0}^{z = 1} p ( {d}_{b} , d , ϕ , Z = z \vert π , t , ρ ) = ( 1 - ρ ) {\prod }_{s = 1}^{S} {\int }_{0}^{1} Pr ( {d}_{b} , d \vert π , ϕ , t ) p ( ϕ \vert Z = 0 ) d ϕ + ρ {\prod }_{s = 1}^{S} {\int }_{0}^{1} Pr ( {d}_{b} , d \vert π , ϕ , t ) P ( ϕ \vert Z = 1 ) d ϕ \qquad ( 10 )$$

for multiple samples (without the product over samples for a single sample).

Ultimately, the quantity that we wish to estimate is the probability of a mutation being clonal (probability that Z=1), in view of the reads observed (d_{b}, d), a genotype prior (π), a tumour fraction estimate (t), and a prior probability of the mutation being clonal (ρ, i.e. we want to estimate P(Z=1|d_{b}, d, π, t, ρ)). In view of the above, this can be expressed as:

$$p ( Z = z \vert {d}_{b} , d , π , t , ρ ) = \frac{p ( {d}_{b} , d , Z = z \vert π , t , ρ )}{p ( {d}_{b} , d \vert π , t , ρ )} \qquad ( 11 )$$

where p(d_{b}, d|π, t, ρ) is given by equation (10) and p(d_{b}, d Z=z|π, t, ρ) is given by equations (9)/(9a). Thus, equation (11) can be written for Z=1 as equation (11a) below:

$$p ( Z = 1 \vert {d}_{b} , d , π , t , ρ ) = \frac{ρ {\prod }_{s = 1}^{S} {\int }_{0}^{1} Pr ( {d}_{b} , d \vert π , ϕ , t ) P ( ϕ \vert Z = 1 ) d ϕ}{( 1 - ρ ) {\prod }_{s = 1}^{S} {\int }_{0}^{1} Pr ( {d}_{b} , d \vert π , ϕ , t ) p ( ϕ \vert Z = 0 ) d ϕ + ρ {\prod }_{s = 1}^{S} {\int }_{0}^{1} Pr ( {d}_{b} , d \vert π , ϕ , t ) P ( ϕ \vert Z = 1 ) d ϕ} \qquad ( 11 a )$$

where ρ is a parameter (set to 0.5 in the examples below), p(ϕ|Z=z) is given by the beta distributions in equation (6), and Pr(d_{b}, d|π, ϕ, t) is given by equations (3)/(4) (one joint genotype) or (3a)/(4a) (plurality of candidate joint genotypes with prior probabilities π).

Thus, estimating equation (11) for z=1 (i.e. equation (11a)) gives us the probability that a mutation is ubiquitous (i.e. assumed to be clonal in view of the one or more samples available).

This requires evaluating S one dimensional integrals (one for each sample, in equations (9), (10)), which can be done efficiently using known numerical integration. Any numerical integration algorithm known in the art may be used for this purpose. For example, a grid approximation may be used. This is advantageously simple, and sufficient considering that there is a single parameter (ϕ) to integrate over.

This provides an estimate of the probability that a mutation is clonal in view of the data available, which can be efficiently computed, is readily interpretable (in view of the rigorous statistical model making use of explicit clear assumptions), can be obtained for any mutation without manual input, is independent of any other mutation analysed, can rigorously include prior knowledge about the mutation, and can be used to objectively and automatically prioritise a list of mutations (with accompanying probabilities) for testing and/or use.

### Accounting for Uncertainty in Copy Number Predictions

While the model described above already presents numerous advantages, it can be further enhanced by taking into account uncertainties in the prediction of the copy number estimates used in the model. Indeed, the above model assumes that the copy numbers (e.g. the major/minor/total/copy numbers used to elicit the genotype priors) were accurately predicted. In practice there may be some uncertainty in these values. Indeed, the problem of allele-specific copy number analysis of tumours is complex and many solutions have been proposed to do this. One commonly used approach is ASCAT (allele-specific copy number analysis of tumors, Van Loo et al., 2010), which takes into account both aneuploidy of the tumour cells and non-aberrant cell infiltration in interpreting a bulk copy number profile, and outputs estimated allele-specific copy number profiles and accompanying tumour purity estimates. In short, ASCAT evaluates a plurality of possible combinations of tumour ploidy and tumour fractions, based on the assumption that the associated allele-specific copy number calls should be as close as possible to nonnegative whole numbers for germline heterozygous single nucleotide polymorphisms (SNPs). A solution deemed optimal is then reported (estimated tumour ploidy, tumour purity and allele-specific copy number calls for the tumour and normal part of the sample) together with its goodness-of-fit (based on the above assumption).

The model provided above can be adjusted to accommodate multiple copy number solutions and their uncertainties, by modifying π to contain entries for the genotypes from each predicted copy number state (e.g. each proposed solution comprising a major and minor copy state), weighted by the probability associated with this state. Additionally, as the tumour purity estimate may be estimated together with these copy number states (as is the case e.g. when an approach like ASCAT is used), the associated tumour purity estimate can also be taken into account. Note that this may not be necessary when e.g. the tumour purity is estimated or measured separately and is not intrinsically associated with the copy number state estimate. Nevertheless, for the sake of generality, let us assume that we have a set of C possible copy number/tumour content states (e.g. C possible sets of estimates of cₘₐⱼₒᵣ, cₘᵢₙₒᵣ, and t). Let π_{C} be a vector where each entry is the probability for each possible such set of estimates. For each state C, it is possible to compute the vector π_{CG} of possible genotypes as explained above. A final genotype vector can thus be obtained by multiplying π_{CG} by the entry for state C in π_{C}. This gives rise to the slightly modified equations below:

$$P ( d , {d}_{b} \vert π , ϕ ) = {Σ}_{i} {π}_{i} Binomial ( {d}_{b} \vert d , ξ ( {G}_{i} , ϕ , {t}_{i} ) ) \qquad ( 3 b )$$

$$P ( d , {d}_{b} \vert π , ϕ , y ) = {Σ}_{i} {π}_{i} Beta Binomial ( {d}_{b} \vert d , ξ ( {G}_{i} , ϕ , {t}_{i} ) , y ) . \qquad ( 4 b )$$

where the tumour content tᵢ may now depend on the particular state (and the πᵢ are elements of the vector π obtained by multiplying π_{CG} by the entry for state C in π_{C}). These new densities can be substituted in the relevant equations above. In particular, the problem solved may then be expressed as solving equation (11a), where Pr(d_{b}, d|π, ϕ, t) is given by equation (3b) or equation (4b). The values for tᵢ, cₘₐⱼₒᵣ, cₘᵢₙₒᵣ (and hence the compatible π_{CG} according to the model used) and π_{C} are provided as outputs of many methods for performing allele-specific copy number analysis of tumours, including but not limited to ASCAT, as explained above. For the avoidance of any doubt, any approach that generates allele-specific copy number state estimates (typically with associated with a tumour purity estimate) with a confidence or other metric that can be used to weight multiple solutions relative to each other may be used for this purpose.

### Implementation

The methods described herein may be implemented using any programming language known in the art. In the examples below, a Python script implementing the above method was used. This took as input, for each mutation: a mutation identifier, a sample identifier, a count of the number of reads that match the reference allele at the mutation position, a count of the number of reads that match the alternate allele at the mutation position, and, for each of one or more copy number solutions: the major copy number (for the tumour) overlapping the mutation for the specified copy number solution, the minor copy number (for the tumour) overlapping the mutation for the specified copy number solution, a copy number for the normal cell at the mutation (may be set to default=2 for autosomal chromosomes, or 1 for a sex chromosome in a male subject), and a tumour purity value for the specified copy number solution (this can also be obtained as an output of e.g. ASCAT, or can be separately obtained). The major and minor copy number overlapping the mutation for the tumour population, for a specified copy number solution, can be obtained directly from ASCAT (e.g. using ascatNgs, Raine et al., 2016), or derived from the output of e.g. ASCAT such as using the mean B allele frequency of the copy number segment overlapping the mutation, the log R value of the copy number segment overlapping the mutation, and the ploidy of the solution. For example, the allele specific copy number estimates (\hat{n}_{A},\hat{n}_{B}) for the tumour at a location i can be expressed as functions of the log R value rat location i, the B allele fraction value b at location i, the ploidy estimate ψ, the tumour cell fraction estimate ρ, and a platform-dependent “technology” parameter t (which can be set to t=1 for next generation sequencing data such as WES) using:

$$= ( ρ - 1 + {2}^{\frac{r}{γ}} ( 1 - b ) ( 2 ( 1 - ρ ) + ρψ ) ) / ρ and \hat{{n}_{B}} = ( ρ - 1 + {2}^{\frac{r}{γ}} b ( 2 ( 1 - ρ ) + ρψ ) ) / ρ .$$

The major and minor copy numbers in the normal population may be assumed to be 1 and 1, apart from mutations on the sex chromosomes which may be handled depending on the sex of the subject. Where multiple copy number solutions are provided, a probability of each solution may optionally be provided (this can also be obtained from the output of e.g. ASCAT which proves a negative log likelihood for a solution). If this is not provided, then all of a plurality of solutions may be treated as equally likely and receive equal weight. The script produced as output a mutation identifier and posterior probability that the mutation is ubiquitous.

In the examples below, whenever a copy number solution was estimated this was done using ASCAT (Van Loo et al., 2010).

### Results

FIG. 11 illustrates the approach that was demonstrated in this example. In particular, the base model described above calculates a posterior probability of a mutation being clonal based on a prior probability of the mutation being clonal expressed as P(Z=z|ρ) where Z is a Bernoulli variable which is one when a mutation is ubiquitous (assumed to be clonal) and zero otherwise, and ρ is the prior probability that the mutation is ubiquitous (set to 0.5 for both Z=1 and Z=0). This is referred to as uninformative prior or neutral prior. In the present model, P(Z=z|ρ) is replaced by a Bernoulli variable that still takes the value of 0 or 1 as a function of a prior probability ρ but instead of being set to a fixed value of 0.5 this is set to a probability that has a value x between 0 and 1 which is set by a fitted logistic regression model or weighted prior model as explained above for z=1, and 1−x for z=0.

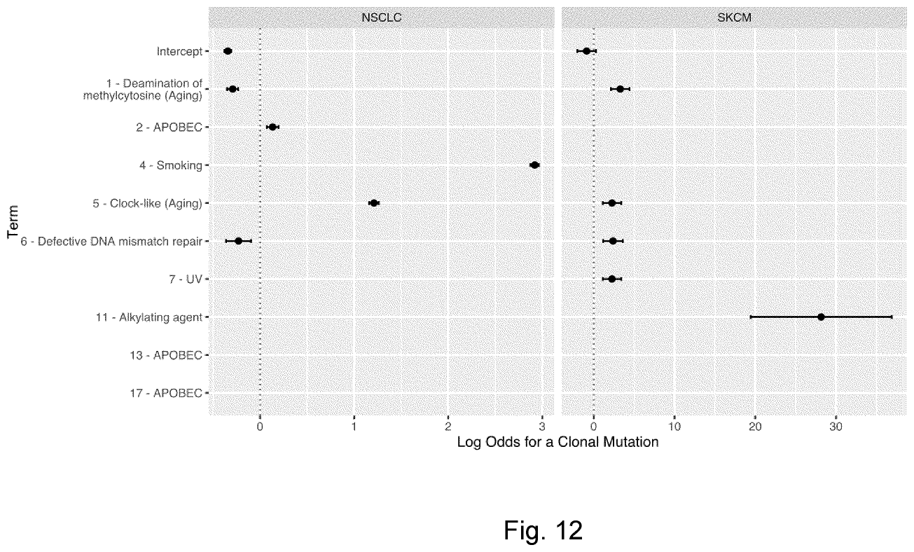

The prior model illustrated on FIG. 11 (logistic regression clonal prior—note that the mutation centric clonal prior illustrated on FIG. 11 shows the prior for z=1) was fitted to NSLC data using COSMIC signatures 1, 2, 4, 5, 6 7, 11, 13 and 17, and to melanoma data from TCGA using the same COSMIC signatures. The resulting distribution of log odds of mutations being clonal in the training data is plotted on FIG. 12 (NSCLC on the left, melanoma on the right). The dashed line in read indicates the value of log odds clonality=0, such that anything on the right of the line indicates that mutations are more likely to be clonal than subclonal and anything on the left indicates that mutations are more likely to be subclonal. This data shows that a mutation that arose from the smoking signature is a strong predictor of clonality in NSCLC, whereas all other signatures are not. Surprisingly, UV exposure was not identified as a strong predictor of clonality in melanoma samples. It was later determine that this is likely because most mutations in this data are assigned to this signature (120419 mutations, out of a total of approx. 130000 mutations), and an unexpectedly large proportion of these (96707 out of 120419) is predicted to be clonal. This is likely due to a phenomenon called “illusion of clonality”, where likelihood of clonality is overestimated in cases where data from only a single sample is available since ubiquity in the sample can be confidently called but is only an imperfect indicator of clonality across the tumour. Thus, this data demonstrates the benefits of training/fitting a model as described herein using multi-sample data. Thus, in the following example only the NSCLC (multi-sample) data was considered.

### Example 3: Use of Mutational Signatures Prior Improves Clonality Calling

### Methods

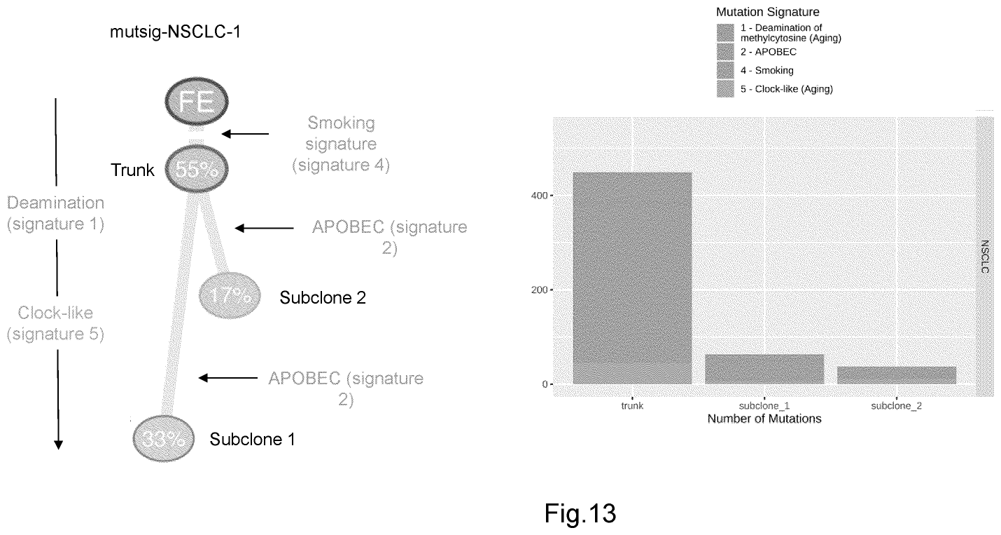

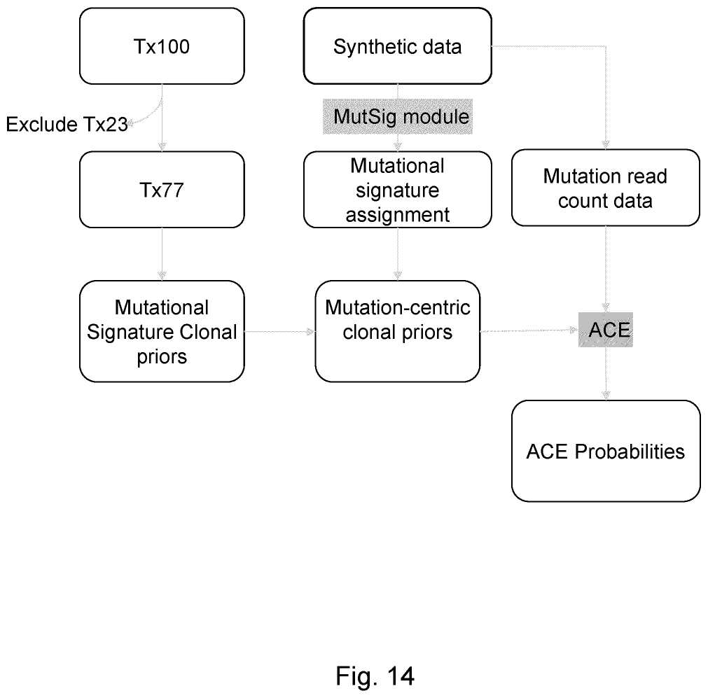

Synthetic data. Synthetic data was obtained by injecting mutations in different branches of a reconstructed phylogeny from data in Salcedo et al. (2020) (in particular, the phylogeny for sample T2 of the DREAM challenge on Somatic Mutation Calling (SMC) Heterogeneity (HET) was used), as indicated on FIG. 13. The left panel on FIG. 13 illustrates the phylogeny used (where FE=founder event). The node percentage represents the proportion of total cells (i.e. tumour and normal cells) that have the mutation and the truncal node percentage is the purity of the sample. Mutations drawn from signature 4 were added prior to branching of the truncal clone, mutations drawn from signature 2 were added in each of the branches. Mutations drawn from signatures 1 and 5 were added throughout the tree. An arbitrary number of mutations were chosen for each signature and then injected into branches based on the typical evolutionary timing of these signatures. Read count data was then obtained by simulating hypothetical variant counts using a binomial where the p parameter was with respect to the expected VAF of each clone (assuming that all mutations are heterozygous). The resulting number of mutations in the trunk and subclones are plotted on the right panel of FIG. 13, showing the activity of signature 4 dominating the mutations in the truncal population, whereas the activity of signature 2 dominates in subclone 1. The synthetic data was used to identify mutation-centric clonal priors (see FIG. 14) based on a logistic regression model and a weighted clonal prior model trained using TRACERx data (see below).

Model training. Data from TRACERx100 was used to train a logistic regression model as explained in example 2 using a subset of 77 samples, as illustrated on FIG. 14. The remaining 23 samples were used for testing (see below).

Validation using TracerX data. The 23 samples not used to train the model were used for testing using an approach as illustrated on FIG. 14.

Validation using TBL data. The approach illustrated on FIG. 14 was also used to assess clonality in an independent cohort of 17 NSCLC patients.

Assessment of simulation results. A custom metric was used to assess the results of the use of informative clonal priors. The metric is referred to as “log loss” and is a binary cross entropy metric that reflects the objective that the posterior probabilities obtained should be closer to 1 for truncal mutations (as there are no deletions in the data) and closer to 0 for subclonal mutations, when adding the prior knowledge. In particular, the metric is expressed as

$${H}_{p} ( q ) = - \frac{1}{N} {\sum }_{i = 1}^{N} {y}_{i} . log ( p ( {y}_{i} ) ) + ( 1 - {y}_{i} ) . ( log ( 1 - p ( {y}_{i} ) ) )$$

where yᵢ is the label of the mutation (1=clonal; 0=subclonal), and p(yᵢ) refers to the clonal posterior probability calculated using the methods described above (ACE). Lower values of this metric are indicative of improved performance.

### Results

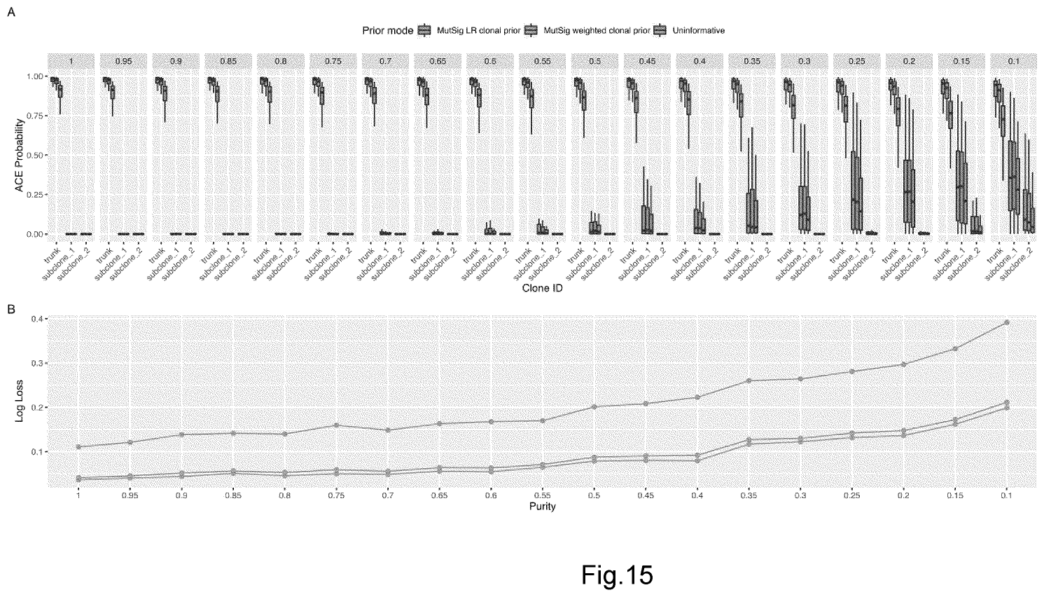

FIG. 15 shows the results of validation on simulated data. FIG. 15A shows the distribution of posterior probabilities of mutations being clonal for mutations that are specific to the trunk (i.e. clonal) and to each of the branches (i.e. subclonal) using each of (a) a logistic regression based prior as described above, (b) a weighted clonal prior as described above, and (c) an uninformative prior. Each plot shows results for data with a different purity. FIG. 15B shows the log loss metric for each of the three approaches (top line=uninformative, middle line=weighted clonal prior, bottom line=logistic regression prior). The data shows that both types of informative priors perform better than the uninformative prior, and that the difference increases as the purity decreases. This reflects the higher importance of the prior in cases where the confidence in the data is low. Note that there is a slight increase of the probabilities for the non-clonal mutations with the informative priors due to the increase in clonal prior overall that reflects the baseline disease clonal rate. Nevertheless, the informative priors still outperform the uninformative priors particularly when it comes to detecting true clonal mutations. Thus, the use of the informative priors as described herein results in an increased sensitivity in detection of clonal mutations.

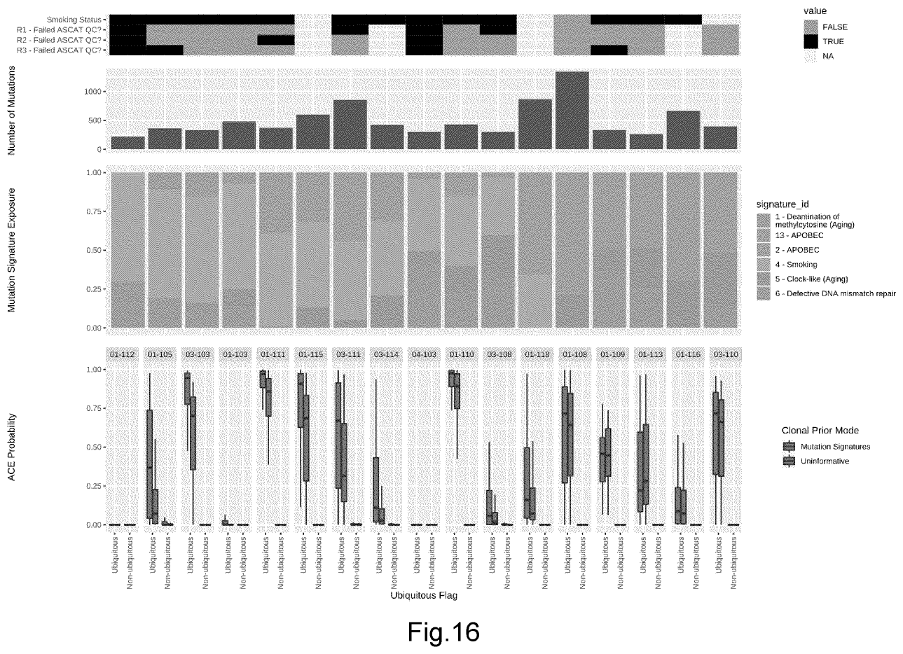

FIG. 16 shows the results of validation on an independent NSCLC cohort. In this case only the logistic regression informative prior (and the comparative uninformative prior model) was tested. The data shows for each sample, the smoking status and whether each of the regions samples failed quality control steps that are associated with low confidence copy number estimates (top row), the number of mutations and the calculated mutation signature weights for the samples (middle rows), and the distribution of probability of clonality for mutations that are ubiquitous (present in all regions samples) and non-ubiquitous (not present in all regions sampled). Ubiquity was used as a surrogate measure of clonality as true clonal labels were not available. The data shows that the weight assigned to the smoking signature correlates well with smoking status, and that the informative prior improved the probabilities for ubiquitous mutations in cases where multiple good quality samples were available. Cases where the smoking status does not match the signature weight assignment (no smoking signature detected) likely result from a situation where the cells that acquired the smoking mutations were not the ones that ultimately formed the primary NSCLC tumour. The few cases on FIG. 16 where no visible improvement in probabilities calculations was observed were found to be samples with low purity and therefore low confidence ASCAT estimates such that the approach correctly was unable to call clonality based on the data available, regardless of the presence of an informative prior.

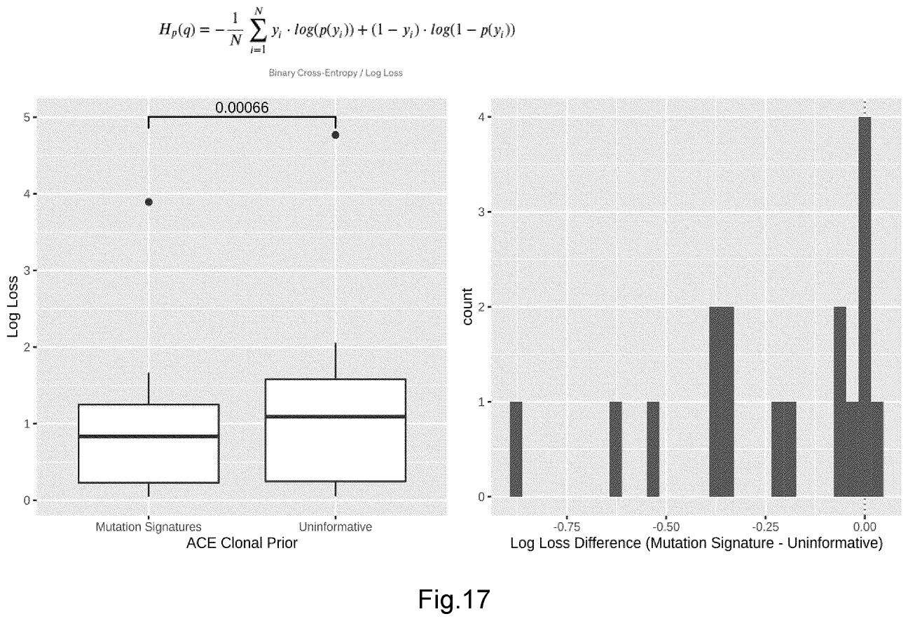

FIG. 17 shows, for the data on FIG. 16, the distribution of the log loss metric for probabilities calculated using an uninformative prior and a logistic regression informative prior (left) and the difference between these two values as a histogram with counts representing patients (right). This data shows that the use of a mutational signature informed prior significantly improves the performance of the calculation of probability of clonality (pairwise log loss difference=0.00066, demonstrating that overall we see a lower log-loss for the informative prior vs. uninformative prior methods). Thus, the data on FIGS. 16-17 shows that there is generally an improvement in clonal probabilities estimation using the mutation signature clonal prior model.

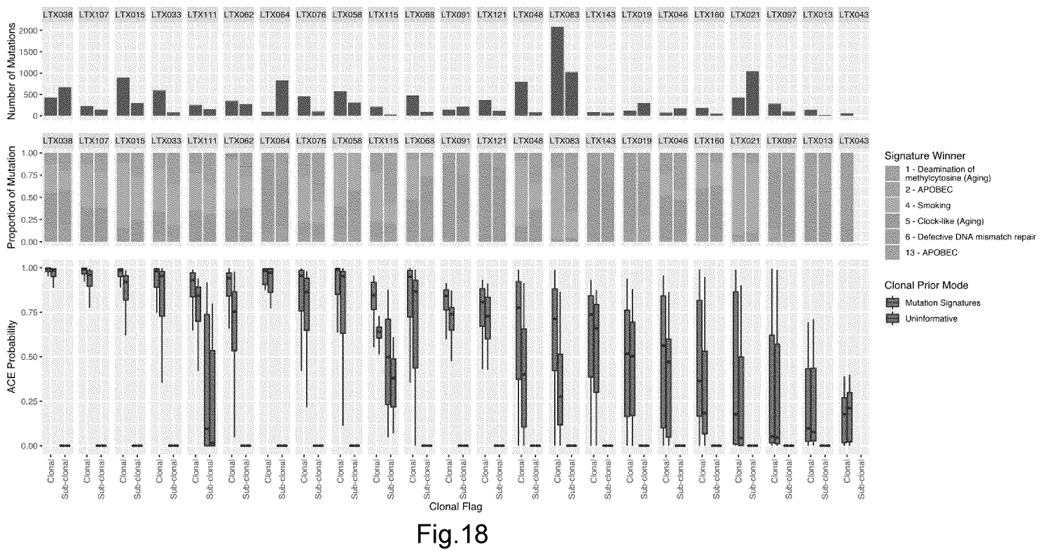

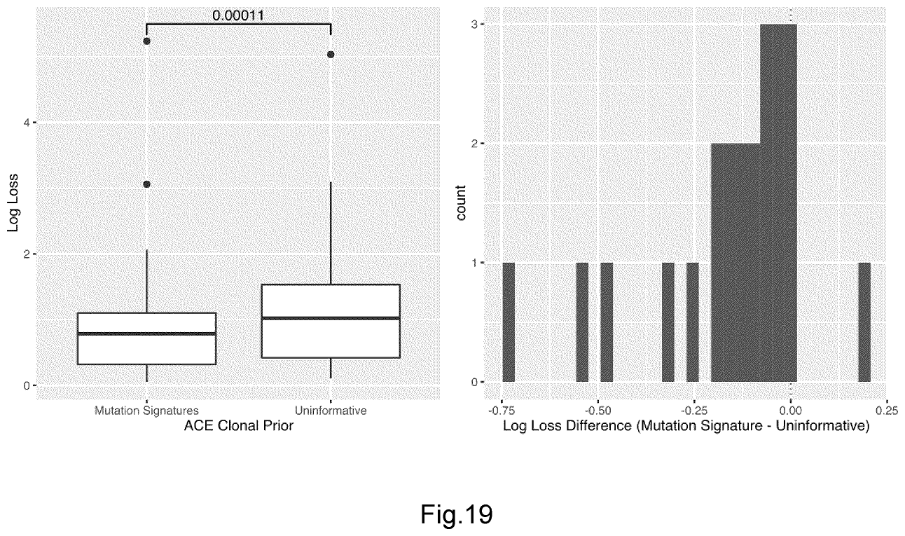

FIGS. 18 and 19 show the results of validation on the subset of TRACERx samples kept aside for training. Again, only the logistic regression informative prior and the uninformative prior were tested. Similar results are shown as on FIGS. 16 and 17. Again, the informative prior significantly increases the ability to correctly identify clonal mutations, in particular in samples that show the activity of a mutational signature that is associated with a strong clonal prior (in this case, the smoking signature, although different signature(s) may be particularly relevant in other types of cancers and/or in cohorts that include patients that have been subject to treatment expected to induce mutations).

### Discussion

These examples demonstrate the development and assessment of a new approach for identifying mutations which are ubiquitously present across cancer cell populations in a patient, namely clonal mutations. This work demonstrates that mutational signatures can be associated with individual mutations and further that this can enable to study connections between mutational signatures and likelihood of clonality, uncovering that mutational signatures can be used as an a priori predictor of clonality. The work further demonstrates that the combination of this knowledge with methods that determine a probability of clonality from sequence data from a tumour enhances the ability of these methods to correctly identify clonal mutations compared to a situation where no a prior knowledge is available. Identifying clonal mutations is a crucial step towards designing therapeutics that target these mutations, and any improvement, even minor, in our ability to identify clonal mutations can make the difference between being able to provide a therapy for a patient and being unable to do so even though a targetable clonal mutation existed, simply because this clonal mutation was not identified. In other words, even moderate improvements in sensitivity of detection of clonal mutations resulting in the additional identification of a handful or even a single candidate clonal mutation can enable to provide a successful therapy for a patient that would have otherwise been untreatable or unsuccessfully treated.

### REFERENCES

Adalsteinsson, V. A., Ha, G., Freeman, S. S. et al. Scalable whole-exome sequencing of cell-free DNA reveals high concordance with metastatic tumors. Nat Commun 8, 1324 (2017).
L. B. Alexandrov et al., Signatures of mutational processes in human cancer. Nature 500, 415-421 (2013).
Alexandrov, L. B., Kim, J., Haradhvala, N. J. et al. The repertoire of mutational signatures in human cancer. Nature 578, 94-101 (2020).
Bergstrom, E. N., Huang, M. N., Mahto, U. et al. SigProfilerMatrixGenerator: a tool for visualizing and exploring patterns of small mutational events. BMC Genomics 20, 685 (2019).
Bulik-Sullivan B, Busby J, Palmer C D, Davis M J, Murphy T, Clark A, Busby M, Duke F, Yang A, Young L, Ojo N C, Caldwell K, Abhyankar J, Boucher T, Hart M G, Makarov V, Montpreville V T, Mercier O, Chan T A, Scagliotti G, Bironzo P, Novello S, Karachaliou N, Rosell R, Anderson I, Gabrail N, Hrom J, Limvarapuss C, Choquette K, Spira A, Rousseau R, Voong C, Rizvi N A, Fadel E, Frattini M, Jooss K, Skoberne M, Francis J, Yelensky R. Deep learning using tumor HLA peptide mass spectrometry datasets improves neoantigen identification. Nat Biotechnol. 2018 Dec. 17.
Carter S L, Cibulskis K, Helman E, McKenna A, Shen H, Zack T, Laird P W, Onofrio R C, Winckler W, Weir B A, Beroukhim R, Pellman D, Levine D A, Lander E S, Meyerson M, Getz G. Absolute quantification of somatic DNA alterations in human cancer. Nat Biotechnol. 2012 May; 30(5):413-21.
de Bruin E C, et al. Spatial and temporal diversity in genomic instability processes defines lung cancer evolution. Science. 2014 Oct. 10; 346(6206):251-6.A. Degasperi et al., A practical framework and online tool for mutational signature analyses show inter-tissue variation and driver dependencies. Nat Cancer 1, 249-263 (2020).
Hossein Farahani, Camila P E de Souza, Raewyn Billings, Damian Yap, Karey Shumansky, Adrian Wan, Daniel Lai, Anne-Marie Mes-Masson, Samuel Aparicio, and Sohrab P Shah. Engineered in-vitro cell line mixtures and robust evaluation of computational methods for clonal decomposition and longitudinal dynamics in cancer. Scientific Reports, 7(1):13467, 2017.
Jamal-Hanjani M, et al. Tracking the Evolution of Non-Small-Cell Lung Cancer. N Engl J Med. 2017 Jun. 1; 376(22):2109-2121.
Vanessa Jurtz, Sinu Paul, Massimo Andreatta, Paolo Marcatili, Bjoern Peters and Morten Nielsen. NetMHCpan-4.0: Improved Peptide-MHC Class I Interaction Predictions Integrating Eluted Ligand and Peptide Binding Affinity Data. J Immunol Nov. 1, 2017, 199 (9) 3360-3368.
Langmead, B., Trapnell, C., Pop, M. et al. Ultrafast and memory-efficient alignment of short DNA sequences to the human genome. Genome Biol 10, R25 (2009).
Landau D A, Carter S L, Stojanov P, McKenna A, Stevenson K, Lawrence M S, Sougnez C, Stewart C, Sivachenko A, Wang L, Wan Y, Zhang W, Shukla S A, Vartanov A, Fernandes S M, Saksena G, Cibulskis K, Tesar B, Gabriel S, Hacohen N, Meyerson M, Lander E S, Neuberg D, Brown J R, Getz G, Wu C J. Evolution and impact of subclonal mutations in chronic lymphocytic leukemia. Cell. 2013 Feb. 14; 152(4):714-26. doi: 10.1016/j.cell.2013.01.019.
Li, S., Crawford, F. W. & Gerstein, M. B. Using sigLASSO to optimize cancer mutation signatures jointly with sampling likelihood. Nat Commun 11, 3575 (2020).
Constance H. Li, Syed Haider, Paul C. Boutros. Ancestry Influences on the Molecular Presentation of Tumours. bioRxiv 2020.08.02.233528; 2020.
Li, C. H., Haider, S. & Boutros, P. C. Age influences on the molecular presentation of tumours. Nat Commun 13, 208 (2022).
Litchfield K, Reading J L, Puttick C, Thakkar K, Abbosh C, Bentham R, Watkins T B K, Rosenthal R, Biswas D, Rowan A, Lim E, Al Bakir M, Turati V, Guerra-Assunção J A, Conde L, Furness A J S, Saini S K, Hadrup S R, Herrero J, Lee S H, Van Loo P, Enver T, Larkin J, Hellmann M D, Turajlic S, Quezada S A, McGranahan N, Swanton C. Meta-analysis of tumor- and T cell-intrinsic mechanisms of sensitization to checkpoint inhibition. Cell. 2021 Feb. 4; 184(3):596-614.e14.
Lundegaard C, Lamberth K, Harndahl M, Buus S, Lund O, Nielsen M. NetMHC-3.0: accurate web accessible predictions of human, mouse and monkey MHC class I affinities for peptides of length 8-11. Nucleic Acids Res. 2008 Jul. 1; 36(Web Server issue):W509-12.
J. Ma, J. Setton, N. Y. Lee, N. Riaz, S. N. Powell, The therapeutic significance of mutational signatures from DNA repair deficiency in cancer. Nat Commun 9, 3292 (2018).
Nicholas McGranahan, Francesco Favero, Elza C de Bruin, Nicolai Juul Birkbak, Zoltan Szallasi, and Charles Swanton. Clonal status of actionable driver events and the timing of mutational processes in cancer evolution. Science translational medicine, 7(283):283ra54-283ra54, 2015.
McGranahan, N., Furness, A. J., Rosenthal, R., Ramskov, S., Lyngaa, R., Saini, S. K., Jamal-Hanjani, M., Wilson, G. A., Birkbak, N. J., Hiley, C. T., Watkins, T. B., Shafi, S., Murugaesu, N., Mitter, R., Akarca, A. U., Linares, J., Marafioti, T., Henry, J. Y., Van Allen, E. M., Miao, D., . . . Swanton, C. (2016). Clonal neoantigens elicit T cell immunoreactivity and sensitivity to immune checkpoint blockade. Science (New York, N. Y.), 351(6280), 1463-1469.
S. Nik-Zainal et al., Landscape of somatic mutations in 560 breast cancer whole-genome sequences. Nature 534, 47-54 (2016).
Timothy J. O'Donnell, Alex Rubinsteyn, Maria Bonsack, Angelika B. Riemer, Uri Laserson, Jeff Hammerbacher. MHCflurry: Open-Source Class I MHC Binding Affinity Prediction. Cell Systems Vol. 7, Issue 1, 129-132, Jul. 25, 2018.
Russell Schwartz and Alejandro A Schsffer. The evolution of tumour phylogenetics: principles and practice. Nature Reviews Genetics, 18(4):213, 2017.
Raine K M, Van Loo P, Wedge D C, Jones D, Menzies A, Butler A P, Teague J W, Tarpey P, Nik-Zainal S, Campbell P J. ascatNgs: Identifying Somatically Acquired Copy-Number Alterations from Whole-Genome Sequencing Data. Curr Protoc Bioinformatics. 2016 Dec. 8; 56:15.9.1-15.9.17. doi: 10.1002/cpbi.17.
Rosenthal, R., McGranahan, N., Herrero, J. et al. deconstructSigs: delineating mutational processes in single tumors distinguishes DNA repair deficiencies and patterns of carcinoma evolution. Genome Biol 17, 31 (2016).
Rosenthal, R., Cadieux, E. L., Salgado, R. et al. Neoantigen-directed immune escape in lung cancer evolution. Nature 567, 479-485 (2019).
Andrew Roth, Jaswinder Khattra, Damian Yap, Adrian Wan, Emma Laks, Justina Biele, Gavin Ha, Samuel Aparicio, Alexandre Bouchard-Ct6, and Sohrab P Shah. PyClone: statistical inference of clonal population structure in cancer. Nature methods, 11(4):396, 2014.
Rustad, E. H., Nadeu, F., Angelopoulos, N. et al. mmsig: a fitting approach to accurately identify somatic mutational signatures in hematological malignancies. Commun Biol 4, 424 (2021).
Van Loo P, Nordgard S H, Lingjmrde O C, Russnes H G, Rye I H, Sun W, Weigman V J, Marynen P, Zetterberg A, Naume B, Perou C M, Borresen-Dale A L, Kristensen V N. Allele-specific copy number analysis of tumors. Proc Natl Acad Sci USA. 2010 Sep. 28; 107(39):16910-5.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Various modifications and variations of the described compositions, methods, and uses of the technology will be apparent to those skilled in the art without departing from the scope and spirit of the technology as described. Any sub-titles herein are included for convenience only and are not to be construed as limiting the disclosure in any way.

The methods of any embodiments described herein may be provided as computer programs or as computer program products or computer readable media carrying a computer program which is arranged, when run on a computer, to perform the method(s) described above.

Unless context dictates otherwise, the descriptions and definitions of the features set out above are not limited to any particular aspect or embodiment of the invention and apply equally to all aspects and embodiments which are described.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

Other aspects and embodiments of the invention provide the aspects and embodiments described above with the term “comprising” replaced by the term “consisting of” or “consisting essentially of”, unless the context dictates otherwise.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

1. A method of determining whether a tumour-specific mutation is likely to be clonal in a subject, the method comprising:
determining, by a processor, a metric indicative of the probability that the mutation is clonal by combining evidence from sequence data obtained from the subject and an indication of whether the mutation is associated with one or more predetermined mutational signatures.

2. The method of claim 1, wherein the metric indicative of the probability that a mutation is clonal is a posterior probability, optionally wherein the posterior probability is a posterior probability that the mutation is clonal or a posterior probability of one or more cancer cell fractions for the mutation.

3. The method of any preceding claim, wherein determining a metric indicative of the probability that the mutation is clonal comprises determining a posterior probability based on:
a prior probability of the mutation being clonal that depends on the indication of whether the mutation is associated with the one or more predetermined mutational signatures, and
a probability of observing the sequence data if the mutation is clonal, non-clonal and/or has a particular cancer cell fraction.

4. The method of claim 3, wherein the probability of observing the sequence data comprises probabilities of observing the sequence data if the tumour-specific mutation is (i) clonal and (ii) non-clonal, in view of a tumour fraction for each of the one or more samples and one or more candidate joint genotypes each comprising a genotype at the location of the tumour-specific mutation for a normal population, a reference tumour population that does not comprise the tumour-specific mutation and a variant tumour cell population that comprises the tumour-specific mutation.

5. The method of any preceding claim, further comprising identifying one or more tumour-specific mutations in the subject,
optionally further comprising determining the metric indicative of the probability that a mutation is clonal for each of the one or tumour-specific mutations; and/or identifying a tumour-specific mutation of the one or more tumour-specific mutations as a clonal tumor-specific mutation if the mutation satisfies one or more criteria at least one of which applies to the determined metric.

6. The method of any of preceding claim, wherein the indication of whether the mutation is associated with a mutational signature comprises a weight obtained from a mutational profile for the subject, that quantifies the probability that said mutation was generated by said mutational signature,
optionally wherein said weight is quantified by multiplying the mutational signature proportion for the mutational class to which the mutation belongs by a sample weight quantifying the contribution of the mutational signature to the mutational profile, and/or
wherein the indication of whether the mutation is associated with a mutational signature further comprises a confidence interval around said weight.

7. The method of any of claims 3 to 6, wherein a prior probability of the mutation being clonal is obtained as the output of a model that predicts the prior probability of the mutation being clonal using inputs comprising the indication of whether the mutation is associated with each of the mutational signatures, optionally wherein the model is a linear model, a logistic regression model or a simple linear model.

8. The method of claim 7, wherein the model is trained or fitted using training data comprising, for a plurality of mutations with known clonal status, indications of whether the mutations are associated with each of the mutational signatures.

9. The method of claim 7 or claim 8, wherein the model inputs further comprise one or more predictive variables selected from: variables associated with the ploidy of the mutation, variables associated with the gene in which the mutation is present, variables associated with the subject, variables associated with the tumour, optionally wherein the variables associated with the ploidy of the mutation comprise a genome doubling status, the variables associated with the gene comprise a driver gene status, the variables associated with the subject comprise an ethnicity status, and/or an age of the subject, and/or the variables associated with the tumour comprise a cancer type or subtype.

10. The method of any of claims 7 to 9, wherein the model is a logistic regression model that predicts the log odds of a mutation being clonal as a function of respective weights obtained from a mutational profile for the subject, that quantify the probability that said mutation was generated by said mutational signature, optionally wherein the model has been trained using training data comprising weights (Xₙ) obtained for each of a plurality of mutations from a plurality of training mutational profiles and respective clonal status for each of the plurality of mutations, and/or wherein the logistic regression model comprises a coefficient βₙ for each of the predetermined mutational signatures and training the logistic regression model comprises identifying the value of coefficients (β₀, βₙ) of the logistic regression model.

11. The method of claim 7 or claim 8, wherein the model is a linear model that predicts the probability of a mutation being clonal as a function of respective weights obtained from a mutational profile for the subject, that quantify the probability that said mutation was generated by said mutational signature and a signature specific probability of clonality for each mutational signature, optionally wherein the signature specific probability of clonality is obtained using training data comprising weights (αₙ=Xₙ) obtained for each of a plurality of mutations from a plurality of training mutational profiles and respective clonal status for each of the plurality of mutations, and/or wherein obtaining the signature specific probability of clonality for a mutational signature comprises assigning a single mutational signature to each of a set of mutations in a plurality of training mutational profiles and determining the proportion of mutations assigned to the mutational signature that are clonal.

12. The method of any of claims 7 to 11, wherein the model is specific to a particular cancer type, and/or wherein the model is trained using training data derived from a particular cancer type and/or wherein the method uses a predetermined set of signatures adapted for the cancer type of the subject.

13. The method of any of claims 7 to 12, further comprising obtaining a model that predicts the prior probability of the mutation being clonal using inputs comprising the indication of whether the mutation is associated with each of the mutational signatures, optionally wherein obtaining a model comprises training a model using training data comprising, for a plurality of mutations with known clonal status, indications of whether the mutations are associated with each of the mutational signatures.

14. The method of any preceding claim, wherein the predetermined mutational signatures are consensus mutational signatures, wherein the predetermined mutational signatures are obtained from a mutational signatures database, wherein the predetermined mutational signatures are mutational signatures that are likely to be active in the tumour of the subject, wherein the predetermined mutational signatures are mutational signatures associated with a known aetiology, wherein the mutational signatures are mutational signatures that are associated with a mutational process that is active in at least some tumours of the same type as the tumour in the subject, wherein the method further comprises selecting the predetermined signatures, wherein the subject is a lung cancer subject or a melanoma subject, and/or wherein the mutational signatures are selected from COSMIC signatures 1, 2, 4, 5, 6, 7, 11, 13 and 17.

15. The method of any preceding claim, wherein the sequence data comprises sequencing reads, and/or wherein the sequence data comprises a count of reads supporting the mutated allele, a count of reads supporting the germline allele(s), and/or the total count of reads, at the genomic location of the tumour-specific mutation.

16. A method of providing a prognosis for a subject that has been diagnosed as having cancer, the method comprising identifying a plurality of tumour-specific mutations in one or more samples from the subject and determining the likelihood of each of the tumour-specific mutations being clonal using the method of any of claims 1 to 15.

17. A method of providing an immunotherapy for a subject that has been diagnosed as having cancer, the method comprising:
identifying one or more clonal neoantigens for the subject, using a method comprising:
identifying a plurality of tumour-specific mutations in the subject;
determining whether one or more of the tumour-specific mutations is likely to be clonal in the subject using the method of any preceding claim; and
determining whether one or more of the tumour-specific mutations is likely to give rise to a neoantigen, wherein a clonal neoantigen is a tumour-specific mutation that satisfies one or more predetermined criteria on whether the tumour-specific mutation is likely to be clonal and one or more criteria on whether the tumour-specific mutation is likely to give rise to a neoantigen; and
designing an immunotherapy that targets one or more of the clonal neoantigens identified.

18. The method of claim 17, wherein the immunotherapy that targets the one or more of the clonal neoantigens is an immunogenic composition, a composition comprising immune cells or a therapeutic antibody.

19. The method of claim 18, wherein the immunotherapy is a composition comprising T cells that recognise at least one of the one or more of the clonal neoantigens identified.

20. The method of claim 19, wherein said composition is enriched for T cells that target at least one of the one or more of the clonal neoantigens identified, optionally wherein the method comprises obtaining a population of T cells and expanding the population of T cells to increase the number or relative proportion of T cells that target at least one of the one or more of the clonal neoantigens identified.

21. A composition comprising a population of T cells obtained or obtainable by the method according to claim 20.

22. A method of treating a subject that has been diagnosed as having cancer, the method comprising administering an immunotherapy that has been provided using the method of claims 17 to 20, or the composition of claim 21.

23. A system comprising:
a processor; and
a computer readable medium comprising instructions that, when executed by the processor, cause the processor to perform the steps of the method of any of claims 1 to 20.

24. One or more non-transitory computer readable media comprising instructions that, when executed by one or more processors, cause the one or more processors to perform the steps of the method of any of claims 1 to 20.
